(19) United States
(12) Patent Application Publication
(10) Pub. No.: US 2024/0216316 A1
(43) Pub. Date: Jul. 4, 2024

(54) DIHOMO-GAMMA LINOLENIC ACID (DGLA) IS A NOVEL SENOLYTIC

(71) Applicant: Buck Institute for Research on Aging, Novato, CA (US)

(72) Inventors: Christopher D. Wiley, Novato, CA (US); Judith Campisi, Novato, CA (US)

(21) Appl. No.: 18/008,135

(22) PCT Filed: Jun. 1, 2021
(86) PCT No.: PCT/US2021/035271

§ 371 Date: Dec. 2, 2022

Related U.S. Application Data
(60) Provisional application No. 63/148,094, filed on Feb. 10, 2021.
(60) Provisional application No. 63/033,739, filed on Jun. 2, 2020.

(51) Int. Cl. A61K 31/202; A61K 45/06

(52) U.S. Cl. CPC A61K 31/202; A61K 45/06

(57) ABSTRACT

Methods are provided that relate to the discovery that dihomo-gamma linolenic acid (DGLA) is a potent senolytic agent. Accordingly, in certain embodiments, methods of selectively killing one or more senescent cells in a subject in need thereof are provided wherein the method(s) involves administering to the subject an effective amount of DGLA.

### CROSS-REFERENCE TO RELATED APPLICATIONS

This application claims benefit of and priority to U.S. Ser. No. 63/148,094, filed on Feb. 10, 2021, and to U.S. Ser. No. 63/033,739, filed on Jun. 2, 2020, both of which are incorporated herein by reference for all purposes.

### STATEMENT OF GOVERNMENTAL SUPPORT

This invention was made with government support under Grant No. R01 AG052744 awarded by the National Institutes of Health. The Government has certain rights in this invention.

### BACKGROUND

Senescent cells increase in tissues and organs of individuals as they age and are found at sites of many age-related pathologies. Senescent cells are believed important for inhibiting the proliferation of dysfunctional or damaged cells and particularly for constraining the development of malignancy (see, e.g., Campisi et al. (2011) Curr. Opin. Genet. Dev., 21: 107-12; Campisi et al. (2001) Trends Cell Biol., 11: S27-31; Prieur et al. (2008) Curr. Opin. Cell Biol., 20: 150-55; and the like). The presence of senescent cells in an individual may contribute to aging and aging-related dysfunction (see, e.g., Campisi (2005) Cell, 120: 513-522; Gorgoulis et al. (2019) Cell, 179: 813-827; and the like).

Cellular senescence, as characterized by the increase of senescent cells typically associated with aging and other pathologies, is a multi-faceted response to damage, stress and certain physiological signals that arrests cell proliferation, essentially irreversibly. It also activates the transcription and secretion of numerous pro-inflammatory cytokines, chemokines, growth factors and proteases, termed the Senescence Associated Secretory Phenotype (SASP) (see, e.g., Coppe et al. (2008) PLoS Biol, 6(12): 2853-2868; Wiley et al. (2016) Cell Metab. 23(6): 1013-1021; Basisty et al. (2020) PLoS Biol., 18: e3000599; Kuilman et al. (2010) Genes Dev. 24(22): 2463-2479). In addition to aging, senescent cells increase as a consequence of genotoxic and radiotherapy and/or cytotoxic anti-cancer therapies, and we have previously shown in mice that genetically ablating senescent cells ameliorates many deleterious outcomes of these therapies.

### SUMMARY

In various embodiments, methods and compositions are provided for selectively killing one or more senescent cells in a subject in need thereof. The methods exploit the identification of dihomo-gamma linolenic acid (DGLA) as a senolytic agent capable of selectively depleting senescent cells in a tissue, organ, or organism. In certain embodiments the senolytic agent is capable of selectively/preferentially killing cells having a SASP phenotype.

Various embodiments contemplated herein may include, but need not be limited to, one or more of the following:

Various embodiments contemplated herein may include, but need not be limited to, one or more of the following:

Embodiment 1: A method of selectively killing one or more senescent cells in a subject in need thereof said method comprising:
administering to said subject an effective amount of one or more agents selected from the group consisting of dihomo-gamma-linolenic acid (DGLA), gamma-linolenic acid (GLA), and a delta-5-desaturase inhibitor (D5D inhibitor).

Embodiment 2: The method of embodiment 1, wherein said subject in need thereof is a subject that shows one or more features of aging in the subject, or wherein said subject in need thereof is a subject receiving DNA damaging or cytotoxic therapy, or wherein said subject in need thereof is a subject having a cancer.

Embodiment 3: The method according to any one of embodiments 1-2, wherein said subject does not have a cancer.

Embodiment 4: The method according to any one of embodiments 1-2, wherein said subject has a cancer or pre-cancerous lesions.

Embodiment 5: The method of embodiment 3, wherein said subject has a precancerous lesion.

Embodiment 6: The method according to any one of embodiments 4-5, wherein said subject has a cancer selected from the group consisting of leukemia, a secondary tumor, a solid tumor, acute leukemia, adrenal gland tumor, ameloblastoma, anaplastic carcinoma of the thyroid, angioma, apudoma, argentaffinoma, arrhenoblastoma, ascites tumor, astroblastoma, astrocytoma, ataxia-telangiectasia-associated tumors, basal cell carcinoma, bone cancer, brain tumor, brainstem glioma, breast cancer, Burkitt's lymphoma, cervical cancer, cholangioma, chondroblastoma, chondrosarcoma, chorioblastoma, choriocarcinoma, colon cancer, craniopharyngioma, cystocarcinoma, cystofbroma, cystoma, ductal carcinoma, ductal papilloma, dysgerminoma, encephaloma, endometrial carcinoma, endothelioma, ependymoma, erythroleukemia, Ewing's sarcoma, extra nodal lymphoma, fibro adenoma, fibro sarcoma, follicular cancer of the thyroid, ganglioglioma, gastrinoma cell, glioblastoma multiform, glioma, gonadoblastoma, haemangioblastoma, haemangioendothelioblastoma, haemangioendothelioma, haemangiopericytoma, haematolymphangioma, haemocytoblastoma, haemocytoma, hairy cell leukemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angioforoma, Kaposi sarcoma, kidney tumor, large cell lymphoma, leukemia, lipoma, liver cancer, liver metastases, Lucke carcinoma, lung cancer, lymphadenoma, lymphangioma, lymphocytic leukemia, lymphocytic lymphoma, lymphoedema, lymphoeytoma, lymphoma, malignant mesothelioma, malignant teratoma, mastocytoma, medulloblastome, melanoma, meningioma, mesothelioma, Morton's neuroma, multiple myeloma, myeloid leukemia, myelolipoma, myeloma, myoblastoma, myxoma, nasopharyngeal carcinoma, neuroblastoma, neurofibroma, neuroglioma, neuroma, non-Hodgkin's lymphoma, oligodendroglioma, optic glioma, osteochondroma, osteogenic sarcoma, osteosarcoma, ovarian cancer, pancoast tumor, pancreatic cancer, phaeochromocytoma, plasmacytoma, primary brain tumor, progonoma, prolactinoma, renal cell carcinoma, retinoblastoma, rhabdosarcoma, sarcoma, skin cancer, small cell carcinoma, squamous cell carcinoma, T-cell lymphoma, testicular cancer, thymoma, trophoblastic tumor, and Wilm's tumor.

Embodiment 7: The method according to any one of embodiments 4-6, wherein said method reduces or prevents precancerous lesions.

Embodiment 8: The method according to any one of embodiments 4-7, wherein said method reduces tumor size or burden.

Embodiment 9: The method according to any one of embodiments 4-8, wherein said method slows or stops the progression of a cancer.

Embodiment 10: The method according to any one of embodiments 4-9, wherein said method eliminates a cancer.

Embodiment 11: The method according to any one of embodiments 4-10, wherein said method reduces or stops metastasis.

Embodiment 12: The method according to any one of embodiments 4-11, wherein said method eliminates cancer cells that have been pushed to senescence.

Embodiment 13: The method according to any one of embodiments 1-2, wherein said subject has received or is receiving or will receive a DNA damaging or cytotoxic therapy.

Embodiment 14: The method of embodiment 13, wherein said DNA damaging therapy or cytotoxic therapy comprises a treatment for cancer.

Embodiment 15: The method of embodiments 13-14, wherein said DNA damaging or cytotoxic therapy comprises a treatment for a cancer selected from the group consisting of leukemia, a secondary tumor, a solid tumor, acute leukemia, adrenal gland tumor, ameloblastoma, anaplastic carcinoma of the thyroid, angioma, apudoma, argentaffinoma, arrhenoblastoma, ascites tumor, astroblastoma, astrocytoma, ataxia-telangiectasia-associated tumors, basal cell carcinoma, bone cancer, brain tumor, brainstem glioma, breast cancer, Burkitt's lymphoma, cervical cancer, cholangioma, chondroblastoma, chondrosarcoma, chorioblastoma, choriocarcinoma, colon cancer, craniopharyngioma, cystocarcinoma, cystofbroma, cystoma, ductal carcinoma, ductal papilloma, dysgerminoma, encephaloma, endometrial carcinoma, endothelioma, ependymoma, erythroleukemia, Ewing's sarcoma, extra nodal lymphoma, fibro adenoma, fibro sarcoma, follicular cancer of the thyroid, ganglioglioma, gastrinoma cell, glioblastoma multiform, glioma, gonadoblastoma, haemangioblastoma, haemangioendothelioblastoma, haemangioendothelioma, haemangiopericytoma, haematolymphangioma, haemocytoblastoma, haemocytoma, hairy cell leukemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angioforoma, Kaposi sarcoma, kidney tumor, large cell lymphoma, leukemia, lipoma, liver cancer, liver metastases, Lucke carcinoma, lung cancer, lymphadenoma, lymphangioma, lymphocytic leukemia, lymphocytic lymphoma, lymphoedema, lymphoeytoma, lymphoma, malignant mesothelioma, malignant teratoma, mastocytoma, medulloblastome, melanoma, meningioma, mesothelioma, Morton's neuroma, multiple myeloma, myeloid leukemia, myelolipoma, myeloma, myoblastoma, myxoma, nasopharyngeal carcinoma, neuroblastoma, neurofibroma, neuroglioma, neuroma, non-Hodgkin's lymphoma, oligodendroglioma, optic glioma, osteochondroma, osteogenic sarcoma, osteosarcoma, ovarian cancer, pancoast tumor, pancreatic cancer, phaeochromocytoma, plasmacytoma, primary brain tumor, progonoma, prolactinoma, renal cell carcinoma, retinoblastoma, rhabdosarcoma, sarcoma, skin cancer, small cell carcinoma, squamous cell carcinoma, T-cell lymphoma, testicular cancer, thymoma, trophoblastic tumor, and Wilm's tumor.

Embodiment 16: The method according to any one of embodiments 13-15, wherein the administration of said agents is an adjunct therapy to said treatment for cancer.

Embodiment 17: The method according to any one of embodiments 13-16, wherein said DNA damaging therapy and/or cytotoxic therapy is selected from the group consisting of irradiation, alkylating agents such as nitrogen mustards (chlorambucil, cyclophosphamide, ifosfamide, melphalan), nitrosoureas (streptozocin, carmustine, lomustine), alkyl sulfonates (busulfan), triazines (dacarbazine, temozolomide) and ethylenimines (thiotepa, altretamine), platinum drugs such as cisplatin, carboplatin, oxalaplatin, antimetabolites such as 5-fluorouracil, 6-mercaptopurine, capecitabine, cladribine, clofarabine, cytarabine, floxuridine, fludarabine, gemcitabine, hydroxyurea, methotrexate, pemetrexed, pentostatin, thioguanine, anthracyclines such as daunorubicin, doxorubicin, epirubicin, idarubicin, anti-tumor antibiotics such as actinomycin-D, bleomycin, mitomycin-C, topoisomerase inhibitors such as topoisomerase I inhibitors (topotecan, irinotecan) and topoisomerase II inhibitors (etoposide, teniposide, mitoxantrone), mitotic inhibitors such as taxanes (paclitaxel, docetaxel), epothilones (ixabepilone), vinca alkaloids (vinblastine, vincristine, vinorelbine), estramustine, cyclin-dependent kinase inhibitors (roscovitine, palbociclib, abemaciclib, olaparib), epigenetic modifiers (curcumin, valproic acid), and HIV medications such as NRTIs (Nucleoside Reverse Transcriptase Inhibitors), NNRTIs (Non-Nucleoside Reverse Transcriptase Inhibitors), and protease inhibitors (azidothymidine, tenofovir, emtricitabine, abacavir, nevirapine, atazanavir, lopinavir).

Embodiment 18: The method according to any one of embodiments 1-17, wherein said method delays the onset and/or slow or stops the progression of one or more symptoms associated with accumulation of senescent cells from said DNA damaging therapy.

Embodiment 19: The method according to any one of embodiments 1-2, wherein said method delays the onset and/or slow or stops the progression of one or more features of aging in the subject.

Embodiment 20: The method of embodiment 19, wherein said feature of aging is selected from the group consisting of systemic decline of the immune system, muscle atrophy and decreased muscle strength, decreased skin elasticity, delayed wound healing, retinal atrophy, reduced lens transparency, reduced hearing, osteoporosis, sarcopenia, hair graying, skin wrinkling, poor vision, frailty, cognitive impairment, ophthalmic disease, and idiopathic pulmonary fibrosis.

Embodiment 21: The method according to any one of embodiments 1-20, wherein said method reduces the severity and/or ameliorates one or more symptoms and/or delays the onset and/or slows or stops the progression of a senescence-associated disease or disorder.

Embodiment 22: The method of embodiment 21, wherein the senescence-associated disease or disorder is selected from the group consisting of cardiovascular disease, Alzheimer's disease and related dementias, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, and cancer treatment-related disability such as atrophy and fibrosis in various tissues, brain and heart injury, and therapy-related myelodysplastic syndromes, an accelerated aging disease such as progeroid syndromes (i.e. Hutchinson-Gilford progeria syndrome, Werner syndrome, Bloom syndrome, Rothmund-Thomson Syndrome, Cockayne syndrome, trichothiodystrophy, combined xeroderma pigmentosum-Cockayne syndrome, restrictive dermopathy), ataxia telangiectasia, Fanconi anemia, Friedreich's ataxia, dyskeratosis congenital, aplastic anemia, IPF, renal dysfunction, kyphosis, herniated intervertebral disc, frailty, hair loss, hearing loss, vision loss (blindness or impaired vision), muscle fatigue, skin conditions, skin nevi, diabetes, metabolic syndrome, sarcopenia, dermatological conditions (e.g., wrinkles, including superficial fine wrinkles; hyperpigmentation; scars; keloid; dermatitis; psoriasis; eczema (including seborrheic eczema); rosacea; vitiligo; ichthyosis vulgaris; dermatomyositis; and actinic keratosis).

Embodiment 23: The method of embodiment 21, wherein the senescence-associated disease or disorder is a cardiovascular disease selected from the group consisting of atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke.

Embodiment 24: The method of embodiment 23, wherein the senescence-associated disease comprises a cardiovascular disease.

Embodiment 25: The method of embodiment 24, wherein said method comprises ameliorating a symptom selected from the group consisting of irregularity in heart rhythm, age-related cellular hypertrophy, increase in the cross-sectional area of a cardiomyocyte and decrease in cardiac stress tolerance.

Embodiment 26: The method of embodiment 21, wherein the senescence-associated disease comprises osteoarthritis.

Embodiment 27: The method of embodiment 21, wherein the senescence-associated disease comprises atherosclerosis.

Embodiment 28: The method of embodiment 21, wherein the senescence-associated disease comprises a pulmonary disease.

Embodiment 29: The method of embodiment 28, wherein said pulmonary disease is selected from the group consisting of pulmonary fibrosis, chronic obstructive pulmonary disease, asthma, cystic fibrosis, emphysema, bronchiectasis, and age-related loss of pulmonary function.

Embodiment 30: The method of embodiment 21, wherein the senescence-associated disease or disorder is an inflammatory or autoimmune disease or disorder selected from the group consisting of osteoarthritis, osteoporosis, oral mucositis, inflammatory bowel disease, kyphosis, and herniated intervertebral disc.

Embodiment 31: The method of embodiment 21, wherein the senescence-associated disease or disorder is a neurodegenerative disease selected from the group consisting of Alzheimer's disease, Parkinson's disease, Huntington's disease, dementia, mild cognitive impairment, and motor neuron dysfunction.

Embodiment 32: The method of embodiment 21, wherein the senescence-associated disease or disorder comprises a metabolic disease selected from the group consisting of diabetes, diabetic ulcer, metabolic syndrome, and obesity.

Embodiment 33: The method of embodiment 21, wherein the senescence-associated disease comprises an eye disease or disorder selected from the group consisting of macular degeneration, glaucoma, cataracts, presbyopia, and vision loss.

Embodiment 34: The method of embodiment 21, wherein the senescence-associated disease comprises an age-related disorder selected from the group consisting of renal disease, renal failure, frailty, hearing loss, muscle fatigue, skin conditions, skin wound healing, liver fibrosis, pancreatic fibrosis, oral submucosa fibrosis, and sarcopenia.

Embodiment 35: The method of embodiment 21, wherein the senescence-associated disease comprises a dermatological disease or disorder selected from the group consisting of eczema, psoriasis, hyperpigmentation, nevi, rashes, atopic dermatitis, urticaria, diseases and disorders related to photosensitivity or photoaging, rhytides; pruritis; dysesthesia; eczematous eruptions; eosinophilic dermatosis; reactive neutrophilic dermatosis; pemphigus; pemphigoid; immunobullous dermatosis; fibrohistocytic proliferations of skin; cutaneous lymphomas; and cutaneous lupus.

Embodiment 36: The method according to any one of embodiments 1-35, wherein said dihomo-gamma-linolenic acid (DGLA), and/or gamma-linolenic acid (GLA), and/or delta-5-desaturase inhibitor (D5D inhibitor) is administered directly to an organ or tissue that comprises the senescent cells.

Embodiment 37: The method according to any one of embodiments 1-35, wherein said dihomo-gamma-linolenic acid (DGLA), and/or gamma-linolenic acid (GLA), and/or delta-5-desaturase inhibitor (D5D inhibitor) is administered orally.

Embodiment 38: The method according to any one of embodiments 1-35, wherein said dihomo-gamma-linolenic acid (DGLA), and/or gamma-linolenic acid (GLA), and/or delta-5-desaturase inhibitor (D5D inhibitor) is administered systemically.

Embodiment 39: The method according to any one of embodiments 1-35, wherein said dihomo-gamma-linolenic acid (DGLA), and/or gamma-linolenic acid (GLA), and/or delta-5-desaturase inhibitor (D5D inhibitor) is administered topically, transdermally, or intradermally.

Embodiment 40: The method according to any one of embodiments 1-35, wherein said dihomo-gamma-linolenic acid (DGLA), and/or gamma-linolenic acid (GLA), and/or delta-5-desaturase inhibitor (D5D inhibitor) is administered intranasally, by inhalation, intratracheally, or by intubation.

Embodiment 41: The method according to any one of embodiments 1-40, wherein said subject is a human.

Embodiment 42: The method according to any one of embodiments 1-40, wherein said subject is a non-human mammal.

Embodiment 43: The method according to any one of embodiments 1-2, wherein said subject has a pathology characterized by the generation of senescent cells and an inflammatory response.

Embodiment 44: The method of embodiment 43, wherein said pathology comprises kyphosis and/or herniated intervertebral discs, and/or osteoporosis.

Embodiment 45: The method of embodiment 43, wherein said pathology comprises irritable bowel syndrome and/or an inflammatory bowel disease.

Embodiment 46: The method of embodiment 45, wherein said pathology comprises colitis and/or Crohn's disease.

Embodiment 47: The method of embodiment 43, wherein said pathology comprises a pulmonary disease.

Embodiment 48: The method of embodiment 47, wherein said pathology comprise a pathology selected from the group consisting of idiopathic pulmonary fibrosis (IPF), chronic obstructive pulmonary disease (COPD), asthma, cystic fibrosis, bronchiectasis, and emphysema.

Embodiment 49: The method of embodiment 43, wherein said pathology comprises a pathology characterized by fibrosis.

Embodiment 50: The method of embodiment 49, wherein said pathology comprises a pathology selected from the group consisting of renal fibrosis, liver fibrosis, pancreatic fibrosis, cardiac fibrosis, skin wound healing, and oral submucous fibrosis.

Embodiment 51: The method according to any one of embodiments 1-50, wherein said agent comprises DGLA.

Embodiment 52: The method of embodiment 51, wherein said DGLA is provided as DGLA ethyl ester.

Embodiment 53: The method of embodiment 51, wherein said DGLA, wherein said DGLA is provided as DGLA inert lipid.

Embodiment 54: The method according to any one or embodiments 51-53, wherein said DGLA is administered without administration of a D5D inhibitor to said subject (e.g., administered to a subject that is not also administered a D5D inhibitor).

Embodiment 55: The method according to any one or embodiments 51-54, wherein said DGLA is administered without administration of GLA to said subject (e.g., administered to a subject that is not also administered GLA).

Embodiment 56: The method according to any one of embodiments 1-54, wherein said agent comprises gamma linoleic acid (GLA).

Embodiment 57: The method of embodiment 56, wherein said GLA is administered without administration of a D5D inhibitor to said subject (e.g., administered to a subject that is not also administered a D5D inhibitor).

Embodiment 58: The method according to any one of embodiments 1-56, wherein said agent comprises a D5D inhibitor.

Embodiment 59: The method of embodiment 58, wherein said D5D inhibitor comprises an inhibitor selected from the group consisting of iminodibenzyl, iminostilbene. compound 1a, compound 3a, compound 1b, compound 3b, compound 1d, compound 1e, compound 1f, compound 2e, compound 3e, compound 2f, compound 3f, compound as shown in Table 1.

Embodiment 60: The method of embodiment 58, wherein said D5D inhibitor compires an inhibitor selected from the group consisting of any one or more of compounds 1-354 as shown in Table 2, and/or compound 326 described by Takagahara et al.

Embodiment 61: The method of embodiment 58, wherein said D5D inhibitor comprises D5D-IN-326 (2-(2,2,3,3,3-Pentafluoropropoxy)-3-[4-(2,2,2-trifluoroethoxy) phenyl]-5,7-dihydro-3H-pyrrolo[2,3-d]pyrimidine-4,6-dione, CAS No.: 1236767-85-3).

Embodiment 62: The method of embodiment 58, wherein said D5D inhibitor comprises CP 24,879, (4-(3-methylbutoxy)-benzenamine, monohydrochloride).

Embodiment 63: The method of embodiment 58, wherein said D5D inhibitor comprises T3364366 (N-[2-[[3,4-Dihydro-4-oxo-3-[4-(2,2,2-trifluoroethoxy)phenyl]thieno[3,4-d]pyrimidin-2-yl]thio]ethyl]acetamide).

Embodiment 64: The method according to any one of embodiments 1-63, wherein said subject is not diagnosed with and/or under treatment for a pathology characterized by aggregation of a protein selected from the group consisting of Aβ, tau, and alpha-synuclein.

Embodiment 65: The method according to any one of embodiments 1-64, wherein said subject is not under treatment for a neurological pathology.

Embodiment 66: The method according to any one of embodiments 1-65, wherein said subject is not under treatment for a condition selected from the group consisting of Alzheimer's disease and related dementias, amyloid or other cause-mediated mild cognitive impairment (MCI), brain or spinal cord injury (including, but not limited to stroke), Huntingtin's disease, and Parkinson's disease.

Embodiment 67: The method according to any one of embodiments 1-66, wherein said subject is not under treatment for an ophthalmic disorder.

Embodiment 68: The method according to any one of embodiments 1-67, wherein said DGLA is not administered for the treatment of a skin pathology and/or to a subject diagnosed with a skin pathology.

Embodiment 69: The method of embodiment 68, wherein said skin pathology comprises a pathology selected from the group consisting of systemic sclerosis, psoriasis, and eczema.

Embodiment 70: The method according to any one of embodiments 1-69, wherein said DGLA is not administered for the treatment of rheumatoid arthritis (RA), and/or to a subject diagnosed with RA.

Embodiment 71: The method according to any one of embodiments 1-70, wherein said DGLA is not administered for the treatment of polyps in the mouth and/or to a subject diagnosed with polyps in the mouth, and/or to a subject identified as having polyps in the mouth.

Embodiment 72: The method according to any one of embodiments 1-71, wherein said DGLA is not administered for the treatment of high cholesterol and/or other blood fats, and/or to a subject identified as having high cholesterol and/or other blood fats.

Embodiment 73: The method according to any one of embodiments 1-72, wherein said DGLA is not administered for the treatment of heart disease, and/or to a subject identified as having heart disease.

Embodiment 74: The method according to any one of embodiments 1-73,

wherein said DGLA is not administered for the treatment of metabolic syndrome (Syndrome-X), and/or to a subject identified as having metabolic syndrome.

Embodiment 75: The method according to any one of embodiments 1-74, wherein said DGLA is not administered for the treatment of diabetic nerve pain or damage, and/or to a subject identified as having diabetic nerve pain or damage.

Embodiment 76: The method according to any one of embodiments 1-75, wherein said DGLA is not administered for the treatment of attention deficit-hyperactivity disorder (ADHD), and/or to a subject identified as having ADHD.

Embodiment 77: The method according to any one of embodiments 1-76, wherein said DGLA is not administered for the treatment of depression and/or depression after childbirth, and/or to a subject identified as having depression and/or depression after childbirth.

Embodiment 78: The method according to any one of embodiments 1-77, wherein said DGLA is not administered for the treatment of chronic fatigue syndrome (CFS), and/or to a subject identified as having CFS.

Embodiment 79: The method according to any one of embodiments 1-78,

wherein said DGLA is not administered for the treatment of hay fever (allergic rhinitis), and/or to a subject identified as having allergic rhinitis.

Embodiment 80: The method according to any one of embodiments 1-79, wherein said DGLA is not administered to help breast cancer patients respond faster to treatment with the drug tamoxifen.

Embodiment 81: The method according to any one of embodiments 1-80, wherein said DGLA and/or said GLA is not administered as a dietary component or as a nutraceutical.

Embodiment 82: The method according to any one of embodiments 1-81, wherein said DGLA and/or said GLA is not provided as a plant seed, and/or plant seed oil.

Embodiment 83: The method according to any one of embodiments 1-82, wherein said method does not comprise administration of DGLA and/or GLA in conjunction with a D5D inhibitor for treatment of a cancer or precancerous condition.

Embodiment 84: The method according to any one of embodiments 1-83, wherein said method does not comprise administration of DGLA and/or GLA in conjunction with a D5D inhibitor for treatment of an autoimmune condition.

Embodiment 85: The method according to any one of embodiments 1-84, wherein said method does not comprise administration of DGLA and/or GLA in conjunction with a D5D inhibitor for treatment of an inflammatory pathology.

Embodiment 86: The method according to any one of embodiments 1-85,

wherein said DGLA and/or GLA, and/or D5D inhibitor is administered in conjunction with one or more additional senolytic agents.

Embodiment 87: The method according to any one of embodiments 1-86, wherein said additional senolytic agents comprise one or more of a CRYAB inhibitor (e.g., 25-hydroxycholesterol), a senolytic agent described in U.S. Patent Publication Nos: US 2019/0022090, US 2019/0000846, US 2018/0303828, US 2018/0256568, US 2018/0235957, US 2018/0235956, US 2018/0193458, US 2018/0117038, US 2017/0348307, US 2017/0326136, US 2017/0224680, US 2017/0209435, US 2017/0198253, US 2017/0196858, US 2017/0196857, US 2016/0339019, US 2016/0038576, an MDM2 inhibitor (e.g., Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-((1-(hydroxymethyl)cyclopropyl)methoxy)-2-(4-nitrobe-nzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, olaparib, AMG-232, NVP-CGM097, MI-773, CAY10681, CAY10682, Y239-EE, RG-7112, a Boronate, RO-5963, HLI 373, JNJ 26854165, MEL23 MI-773, RG-7112, JNJ 26854165, AD20187), an inhibitor of one or more BCL-2 anti-apoptotic protein family members wherein the inhibitor inhibits at least BCL-xL BCL2 (e.g., ABT-263, ABT-737, WEHI-539, A-1155463, a benzothiazole-hydrazone compound (e.g., WEHI-539), an aminopyridine compound, a benzimidazole compound, a tetrahydroquinolin compound, a phenoxyl compound, and/or an Akt-specific inhibitor (e.g., MK-2206).

### Definitions

A senolytic agent (e.g., dihomo-gamma linolenic acid (DGLA)) as used herein is an agent that “selectively” (preferentially or to a greater degree) destroys, kills, removes, or facilitates selective destruction of senescent cells. In other words, the senolytic agent destroys or kills senescent cells in a biologically, clinically, and/or statistically significant manner compared with its capability to destroy or kill non-senescent cells. Typically, but not necessarily, a senolytic agent is used in an amount and for a time sufficient to selectively kill established senescent cells but insufficient to kill (destroy, cause the death of) non-senescent cells in a clinically significant or biologically significant manner. In certain embodiments, the senolytic agents described herein alter at least one signaling pathway in a manner that induces (initiates, stimulates, triggers, activates, promotes) and results in (i.e., causes, leads to) death of the senescent cells.

When used in the context of the methods provided herein, the term “one or more senolytic agents” refers to the use of dihomo-gamma linolenic acid (DGLA) as described herein, or to the use of a dihomo-gamma linolenic acid (DGLA) as described herein in combination with one or more additional senolytic agents. In certain embodiments, the additional senolytic agents, when present comprise a CRYAB inhibitor (e.g., 25-hydroxycholesterol), and/or other senolytic agents including, but not limited to those described in U.S. Patent Publication Nos: US 2019/0022090, US 2019/0000846, US 2018/0303828, US 2018/0256568, US 2018/0235957, US 2018/0235956, US 2018/0193458, US 2018/0117038, US 2017/0348307, US 2017/0326136, US 2017/0224680, US 2017/0209435, US 2017/0198253, US 2017/0196858, US 2017/0196857, US 2016/0339019, US 2016/0038576, and the like. In certain illustrative embodiments, the additional senolytic agents can include an MDM2 inhibitor (e.g., Nutlin-3a, Nutlin-3b, RG-7112, RG7388, RO5503781, MI-63, MI-126, MI-122, MI-142, MI-147, MI-18, MI-219, MI-220, MI-221, MI-773, 3-(4-chlorophenyl)-3-((1-(hydroxymethyl)cyclopropyl)methoxy)-2-(4-nitrobe-nzyl)isoindolin-1-one, RO-2443, RO-5963, AM-8553, WEHI-539, A-1155463, A-1331852, ABT-263, ABT-199, ABT-737, MK-2206, CCT128930, JNK-IN-8, sanguinarine chloride, methyl 3-(4-nitrophenyl) propiolate (NPP), AT7867, AZD7762, sunitinib, GDC-0980, BKM120, NQDI-1, R406, erlotinib, CYM 7008-00-01, GlcNAc, olaparib, AMG-232, NVP-CGM097, MI-773, CAY10681, CAY10682, Y239-EE, RG-7112, a Boronate, RO-5963, HLI 373, JNJ 26854165, MEL23 MI-773, RG-7112, JNJ 26854165, AD20187, and the like), and/or an inhibitor of one or more BCL-2 anti-apoptotic protein family members wherein the inhibitor inhibits at least BCL-XL BCL2 (e.g., ABT-263, ABT-737, WEHI-539, A-1155463, a benzothiazole-hydrazone compound (e.g., WEHI-539), an aminopyridine compound, a benzimidazole compound, a tetrahydroquinolin compound, a phenoxyl compound, and the like) and/or an Akt-specific inhibitor (e.g., MK-2206).

The phrases “in combination with”, “co-administering”, “concurrent administration”, “administering in conjunction with” or “administering in combination” when used, for example with respect to DGLA and one or more additional senolytic agents refers to administration of DGLA and the one or more additional senolytic agents such that both can simultaneously achieve a physiological effect. The DGLA and the additional senolytic agent(s) need, need not be administered together, either temporally or at the same site. Moreover, DGLA and the additional senolytic agent(s) need not be administered by the same method, e.g., the DGLA may be administered orally and the additional senolytic agent(s) may be administered intravenously. In some embodiments, the DGLA and the additional senolytic agent(s) are administered at different times and, optionally, by different methods of administration. In some embodiments, administration of one can precede administration of the other. Simultaneous physiological effects need not necessarily require the presence of the DGLA and the additional senolytic agent(s) in the circulation at the same time. However, in certain embodiments, co-administering typically results in both the DGLA and the additional senolytic agent(s) being simultaneously present in the body (e.g., in the plasma) at a significant fraction (e.g., 20% or greater, preferably 30% or 40% or greater, more preferably 50% or 60% or greater, most preferably 70% or 80% or 90% or greater) of their maximum serum concentration for any given dose. In some embodiments, DGLA and the additional senolytic agent(s) are administered essentially simultaneously. In some embodiments DGLA and the additional senolytic agent(s) are administered as a combined formulation.

The terms “subject,” “individual,” and “patient” may be used interchangeably and refer to humans, as well as non-human mammals (e.g., non-human primates, canines, equines, felines, porcines, bovines, ungulates, lagomorphs, and the like). In various embodiments, the subject can be a human (e.g., adult male, adult female, adolescent male, adolescent female, male child, female child) under the care of a physician or other health worker in a hospital, as an outpatient, or other clinical context. In certain embodiments, the subject may not be under the care or prescription of a physician or other health worker.

As used herein, the phrase “a subject in need thereof” refers to a subject, as described infra, that is characterized by elevated levels of senescent cells and/or a pathology characterized by elevated levels of senescent cells, and/or undergoing a treatment known to elevate levels of senescent cells.

The term “treat” when used with reference to treating, e.g., a pathology or disease refers to the mitigation and/or elimination of one or more symptoms of that pathology or disease, and/or a delay in the progression and/or a reduction in the rate of onset or severity of one or more symptoms of that pathology or disease, and/or the prevention of that pathology or disease. The term “treat” can refer to prophylactic treatment, which includes a delay in the onset or the prevention of the onset of a pathology or disease.

The term “senolytic agent” when used herein with respect to GLA or a D5D inhibitor does not requires that the GLA or D5D inhibitor itself be seonlygic, but rather that the administration of the GAL and/or D5D inhibitor increased the level of the DGLA which is a senolytic agent.

The term “effective agent(s)” or “agents” or “therapeutic agents” as used herein refers to DGLA, and/or GLA, and/or a D5D inhibitor.

### BRIEF DESCRIPTION OF THE DRAWINGS

FIG. 1 IMR-90 cells were proliferative in 10% serum, made quiescent by incubation for 3 days in 0.2% serum, or made senescent by treatment with 10 Gy X-rays (IR) or ethidium bromide to induce mitochondrial-dysfunction induced senescence (MiDAS). Lipids were extracted from proliferating (PRO—10%), quiescent (QUI—0.2%), IR-induced senescent (SEN(IR)—10% or 0.2% serum), or mitochondrial dysfunction-associated senescent (MiDAS—0.2) cells and analyzed by liquid chromatography combined with mass spectrometry (LC-MS). Lipid moieties were detected in control and senescent cells. Heat maps indicate the averages of 3 experiments (*=p<0.05, 1-way ANOVA).

FIG. 2, panels A-B, shows that DGLA accumulates in senescent cells. Panel A) DGLA was measured in either proliferating (PRO—10% FBS) or irradiation-induced senescent IMR-90 fibroblasts [SEN(IR)—10% FBS] 10 days after treatment, and relative abundances were measured by mass spectrometry and normalized to total protein (BCA assay). Panel B) Cells were cultured as in panel A for 7 days, followed by 3 days in 0.2% FBS to induce quiescence (QUI—0.2% FBS). Senescence caused by mitochondrial dysfunction (MiDAS) was induced by serial passage of IMR-90 fibroblasts in the presence of ethidium bromide to deplete mitochondrial DNA, followed by pyruvate depletion and culture in 0.2% FBS (MiDAS—0.2% FBS).

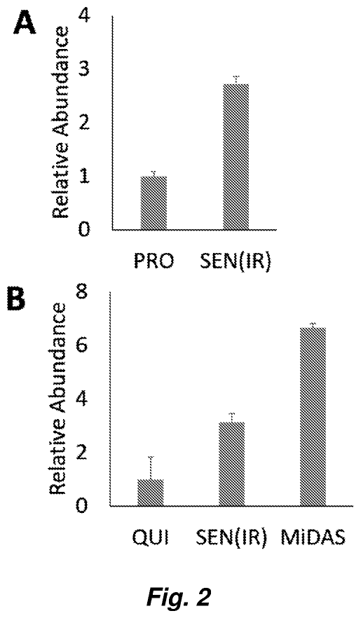

FIG. 3 shows that DGLA is selectively toxic to senescent cells. IMR-90 fibroblasts were either mock-irradiated (Mock) or irradiated with 10Gy of ionization radiation (IR) to induce senescence. 10 days later, cells were cultured in the presence of either vehicle (media plus FBS carrier) or 50 micromolar DGLA for 2 days. Cells were then photographed using light microscopy.

FIG. 4, panels A-B, shows that senescent cells elevate expression of COX-2 (PTGS2) (panel A) and lose 45-desaturase (FADS1) (panel B) expression. IMR90 fibroblasts were induced to senesce by 10 Gy of ionizing radiation [SEN(IR)] or mitochondrial DNA depletion (MiDAS). All treatment groups (senescent or quiescent [QUI]) were cultured for 3 days in 0.2% FBS for 3 days before analysis. RNA was then extracted and analyzed for the indicated genes by quantitative PCR, normalized to actin.

FIG. 5 shows that DGLA is selectively toxic to senescent cells in a COX-2 dependent manner. Cells were either irradiated [SEN(IR)] or mock irradiated (non-senescent or “NonS”) and cultured continuously for 10 days in the presence of either NS-398 (a COX-2 inhibitor) or DMSO (vehicle). Cells were then treated with either carrier (BSA) or DGLA (25 micromolar) for 2 days, and images were captured by light microscopy.

FIG. 6, panels A-B, shows that DGLA induces apoptosis in senescent cells in a COX-2-dependent manner. Cells were treated as in FIG. 5. NS≡Non-senescent proliferating, SEN(IR)=IR-induced senescence, treated with 25 micromolar DGLA and either DMSO or NS-398. Panel A) Percent survival was calculated by cell counts relative to cells treated without DGLA. Panel B) Cells were stained for cleaved caspase 3 by immunofluorescence and expressed as stained cells relative to DAPI positive nuclei.

FIG. 7, panels A-C, shows that DGLA lowers the burden of p16-positive cells. P16-3MR mice were aged for 22 months and luminescence (p16 promoter activity) was measured by luminometry (panel A). Mice were then gavaged with 400 mg/kg of DGLA ethyl ester or vehicle (phosal 50) for 5 consecutive days. Two days after the final gavage, luminescence was again measured (panel B). Six total mice were measured for luminescence and plotted before and after DGLA treatment (panel C).

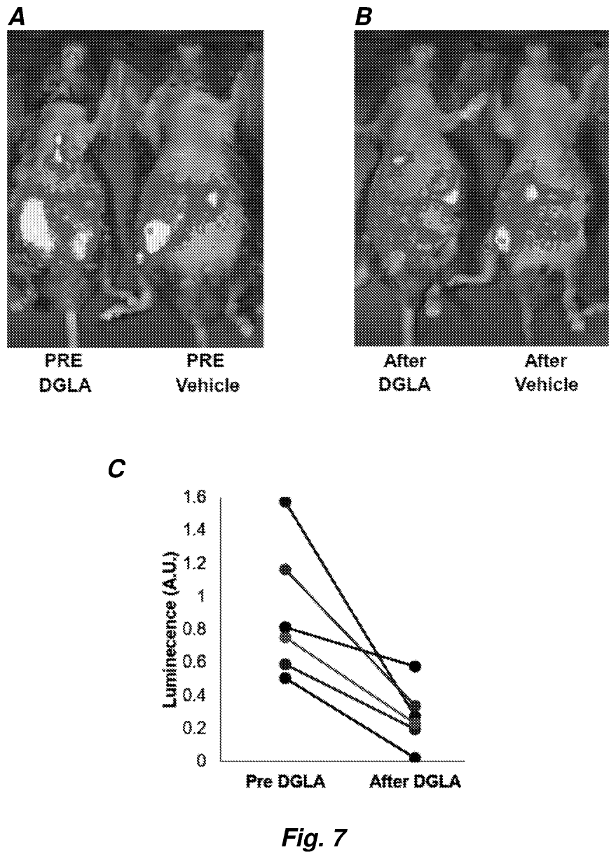

FIG. 8 shows that DGLA lowers the burden of p16-positive cells. P16-3MR mice were aged for 22 months, and gavaged with 400 mg/kg of DGLA ethyl ester or vehicle (phosal 50) for 5 consecutive days. Five days after the final gavage, epididymal fat and liver tissues were isolated and stained for senescence-associated beta-galactosidase.

FIG. 9 shows that inhibition of delta-5 desaturase selectively kills senescent cells treated with 10 PM DGLA/

FIG. 10 shows that T3364366, a D5D-specific inhibitor, selectively kills senescent cells.

FIG. 11 illustrates visualization of senescent cell killing by T334366.

FIG. 12, panels A-B, shows that T3364366 only kills senescent cells in the presence of FBS. Panel A) Senescent vs non-senescent—10% FBS. Panel B) Senescent cells cultured in 10% FBS or 0.2% FBS.

FIG. 13 shows that T3364366 (10 μM) only kills cells in the presence of FBS.

### DETAILED DESCRIPTION

In various embodiments, methods and compositions are provided for selectively killing one or more senescent cells in a subject in need thereof. The methods exploit the identification of dihomo-gamma linolenic acid (DGLA) as a potent senolytic agent. Accordingly, in certain embodiments, methods are provided for treating senescence-associated diseases by administering DGLA alone or in combination with one or more additional senolytic agents. As described herein, in various embodiments the DGLA is administered for a time sufficient and in an amount sufficient to selectively diminish or deplete senescent cells, particularly in one or more target organs of interest.

As explained in Example 1, toxic reactive carbon species such as 8-HOA are made as minority products of DGLA oxygenation by COX-2. In particular, DGLA is peroxidated on carbon 8 by COX-2 as a minority product of the enzyme activity. Beta-scission on either side of this residue results in formation of either a heptanoic acid radical, or an 8-hydroxy-octanoic acid (8-HOA) radical. These are toxic to the cell and induce apoptosis.

In view of this, one potential vulnerability of senescent cells is that they elevate prostaglandin synthase 2 expression (aka COX-2, gene name PTGS2). This is coupled to a loss of 45-desaturase (aka D5D, gene name FADS1), an enzyme that desaturates PUFAs as illustrated.

We found that the endogenous lipid, DGLA, kills senescent cells in a COX-2-dependent manner. Since DGLA is converted to non-toxic arachidonic acid by delta-5-desaturase (D5D) in most normal cells (see, e.g., Nakamura & Nara (2004) Annu. Rev. Nutr. 24: 345-376), the combination of gain of COX-2 and loss of D5D in senescent cells makes this an exploitable weakness. As an endogenous lipid that is only likely to be toxic to senescent cells, DGLA is believed to provide a superior option to most current senolytics. Its derivatives, the 1-series prostaglandins (PGX1's) are largely anti-inflammatory, and therefore may also have positive effects in the context of sterile inflammation associated with aging, so-called “inflammaging” (see, e.g., Franceschi & Campisi (2014) J. Gerontol. A Biol. Sci. Med. Sci. 69 (Suppl 1): S4-9).

Additionally, it is noted that inhibition of D5D causes accumulation of dihomo-gamma-linolenic acid (DGLA). In view of this is believed the use of D5D inhibitors alone or in conjunction with DGLA can provide effective senolytic activity.

It is also recognized that, in certain embodiments, as an alternative to administration of DGLA, or in combination with administration of DGLA, the upstream DGLA precursor gamma linolenic acid (GLA) can be administered to effectively increase DGLA.

Accordingly, in certain embodiments methods are provided for the use of an effective amount of one or more effective agent(s) (DGLA and/or GLA, and/or a D5D inhibitor) to selectively reduce or deplete senescent cells, e.g., particularly, in a tissue or organ, or in the whole organism and thereby to treat or prevent a senescence-associated disease or disorder.

The methods find utility in ameliorating one or more symptoms of senescence-associated and/or age-related diseases and/or slowing the onset and/or progression of senescence-associated and/or age-related diseases. In certain embodiments, the methods find utility in the prevention or treatment of therapy induced senescent cells as described herein. In various embodiments, these methods involve administration an effective amount (dose) of the senolytic agent, DGLA alone or in combination with one or more additional senolytic agents as described herein.

In certain embodiments the agent (e.g., DGLA, and/or GLA, and/or a D5D inhibitor) is used to selectively kill cells undergoing oncogene-induced senescence.

In certain embodiments the agent (e.g., DGLA, and/or GLA, and/or a D5D inhibitor) is used to selectively kill cells undergoing drug-induced senescence.

In certain embodiments the agent (e.g., DGLA, and/or GLA, and/or a D5D inhibitor) is used to selectively kill cells undergoing irradiation-induced senescence.

In certain embodiments the agent (e.g., DGLA, and/or GLA, and/or a D5D inhibitor) is used to treat or prevent any one or more of a wide range of different senescence-associated diseases and disorders. For example, in one illustrative, but non-limiting embodiment, administration of the agent(s) (e.g., DGLA, and/or GLA, and/or a D5D inhibitor) delays (or prevents) tumorigenesis driven by the pro-inflammatory SASP. In this regard it is noted, for example, that some cells can develop a SASP comprising factors that are immunosuppressive and protumorigenic by, e.g., paracrine mechanisms. Likewise, the SASP in certain treated cancers can either contribute to durable responses or drive relapse.

In another illustrative, but non-limiting, embodiment, administration of the agent(s) (e.g., DGLA, and/or GLA, and/or D5D inhibitor) attenuates (the rate and/or extent) of cataract formation. In still another illustrative, but non-limiting embodiment, administration of the agent(s) (e.g., DGLA, and/or GLA, and/or D5D inhibitor) attenuates atherosclerosis. In another illustrative, but non-limiting embodiment, administration of the agent(s) (e.g., DGLA, and/or GLA, and/or D5D inhibitor) attenuates the age-related deterioration of kidney, fat and heart amongst other organs.

In certain embodiments the agent(s) (e.g., DGLA, and/or GLA, and/or D5D inhibitor) are used to treat or prevent a senescence-associated disease or disorder selected from the group consisting of a cancer, cardiovascular disease, Alzheimer's disease and related dementias, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, type 2 diabetes, obesity, fat dysfunction, coronary artery disease, cerebrovascular disease, periodontal disease, and cancer treatment-related disability such as atrophy and fibrosis in various tissues, brain and heart injury, and therapy-related myelodysplastic syndromes, an accelerated aging disease such as progeroid syndromes (i.e. Hutchinson-Gilford progeria syndrome, Werner syndrome, Bloom syndrome, Rothmund-Thomson Syndrome, Cockayne syndrome, xeroderma pigmentosum, trichothiodystrophy, combined xeroderma pigmentosum-Cockayne syndrome, restrictive dermopathy), ataxia telangiectasia, Fanconi anemia, Friedreich's ataxia, dyskeratosis congenital, aplastic anemia, renal dysfunction, kyphosis, herniated intervertebral disc, frailty, hair loss, hearing loss, vision loss (blindness or impaired vision), muscle fatigue, skin conditions, skin nevi, diabetes, metabolic syndrome, sarcopenia, dermatological conditions (e.g., wrinkles, including superficial fine wrinkles; hyperpigmentation; scars; keloid; dermatitis; psoriasis; eczema (including seborrheic eczema); rosacea; vitiligo; ichthyosis vulgaris; dermatomyositis; and actinic keratosis). In certain embodiments the methods described herein expressly exclude the use of DGLA in the treatment and/or prevention of a cancer. In certain embodiments the methods described herein expressly exclude the use of DGLA in the treatment and/or prevention of a cardiovascular condition. The use of the senolytic agent DGLA in various conditions is described below.

Therapeutic and/or Prophylactic Methods

In various embodiments, methods are provided for selectively killing one or more senescent cells in a sample (e.g., in a biological sample), where the method involves contacting the sample with an effective amount of DGLA, and/or GLA, and/or a D5D inhibitor, e.g., as described herein.

In certain embodiments, methods are provided for selectively killing one or more senescent cells in a subject in need thereof, where the method involves contacting the sample with an effective amount of DGLA, and/or GLA, and/or a D5D inhibitor. In certain embodiments, the subject in need thereof is a subject with an age-related disorder. In certain embodiments, the subject in need thereof has a pathology characterized by production of senescent cells and/or an inflammatory response.

By selectively killing one or more senescent cells is meant that the of DGLA, and/or GLA, and/or a D5D inhibitor does not appreciably kill non-senescent cells at the same concentration. Accordingly, in certain embodiments, the median lethal dose or LD₅₀ of the DGLA in non-senescent cells may be about 5 to about 500, or about 5 to about 400, or about 5 to about 300, or about 5 to about 300, or about 5 to about 200, or about 5 to about 100, or about 5 to about 90, or about 5 to about 80, or about 5 to about 70, or about 5 to about 60, or about 5 to about 50 times higher than the LD₅₀ of DGLA, and/or GLA, and/or D5D inhibitor in senescent cells. As used herein, the LD₅₀ is the concentration of DGLA, and/or GLA, and/or D5D inhibitor required to kill half the cells in the cell sample. For example, the LD₅₀ of the of DGLA, and/or GLA, and/or D5D inhibitor in non-senescent cells may be greater than about 5, about 6, about 7, about 8, about 9 or about 10 times higher than the LD₅₀ of the of DGLA, and/or GLA, and/or D5D inhibitor in senescent cells. In certain embodiments, the LD₅₀ of the of DGLA, and/or GLA, and/or D5D inhibitor in non-senescent cells may be greater than about 10, about 15, about 20, about 25, about 30, about 35, about 40, about 45, or about 50 times higher than the LD₅₀ of the DGLA in senescent cells. In certain embodiments, the LD₅₀ of the of DGLA, and/or GLA, and/or D5D inhibitor in non-senescent cells may be greater than about 50, about 100, about 200, about 300, about 400, about 500 times higher than the LD₅₀ of the of DGLA, and/or GLA, and/or D5D inhibitor in senescent cells. In certain embodiments, the LD₅₀ of the of DGLA, and/or GLA, and/or D5D inhibitor in non-senescent cells may be greater than 50 times higher than the LD₅₀ of the of DGLA, and/or GLA, and/or D5D inhibitor in senescent cells. In one illustrative embodiment, the LD₅₀ of the of DGLA, and/or GLA, and/or D5D inhibitor in non-senescent cells is greater than 10 times higher than the LD₅₀ of the of DGLA, and/or GLA, and/or D5D inhibitor in senescent cells. In another illustrative embodiment, the LD₅₀ of the of DGLA, and/or GLA, and/or D5D inhibitor in non-senescent cells is greater than 20 times higher than the LD₅₀ of the corresponding DGLA, and/or GLA, and/or D5D inhibitor in senescent cells.

The progression from an actively dividing cell to a metabolically active, non-dividing cell is termed “senescence” or “cellular senescence.” As used herein, the terms “senescence” and “cellular senescence” may be used interchangeably. The term “senescence” also refers to the state into which cells enter after multiple rounds of division and, as a result of cellular pathways, future cell division is prevented from occurring even though the cell remains metabolically active. Senescent cells may differ from their pre-senescent counterparts in one or more of the following ways: 1) they have arrested growth and cannot be stimulated to reenter the cell cycle by physiological mitogens; 2) they become relatively resistant to apoptotic cell death; and/or 3) they acquire altered differentiated or specialized functions.

In contrast to cancer cells that grow and divide uncontrollably, the ability of most differentiated eukaryotic cells to proliferate is finite. Stated another way, normal cells have an intrinsically determined limit to the number of cell divisions through which they can proceed. This phenomenon has been termed “replicative cellular senescence” and is an intrinsic anticancer mechanism that limits a cell's proliferative ability, thereby preventing neoplastic progression. Another form of senescence is “stress-induced cellular senescence” (sometimes inaccurately termed premature senescence). Stress-induced cellular senescence, like replicative cellular senescence, is a terminal fate of mitotic cells, characterized by permanent cell cycle arrest. Unlike replicative cellular senescence, however, stress-induced cellular senescence does not require telomere deterioration and can be induced by a variety of stressors including, but not limited to, ultraviolet light, reactive oxygen species, certain chemotherapeutics, environmental toxins, cigarette smoking, ionizing radiation, distortion of chromatin structure, excessive mitogenic signaling, and oncogenic mutations. Still another form of senescence is therapy-induced senescence (TIS) which refers to the phenomenon of a subset of cells (e.g., neoplastic cells such as tumor cells) being forced into a senescent state by therapeutic agents. TIS is known to develop because of certain treatments, including radiotherapy and certain chemotherapies such as cancer medications and HIV medications.

The number of senescent cells in various organs and tissues of a subject is known to increase with age. This increase in senescent cells may drive various aspects of the deterioration that underlies aging and age-related diseases. For example, the senescent cells in aged tissue may contribute to age-associated tissue dysfunction, reduced regenerative capacity, and disease. In this context, senescence is considered deleterious because it contributes to decrements in tissue renewal and function. As a non-limiting example, an aged tissue may lack the ability to respond to stress when proliferation is required, thereby resulting in the reduced fitness seen with aging

### Cellular Targets—Senescent Cells

The method described herein involves the specific or preferential killing of senescent cells (e.g., cells expressing a SASP) in a clinically significant or biologically significant manner (e.g., non-senescent cells are not killed or where killed the cell death produces no pathological symptoms). As discussed in detail herein, in various embodiments, the one or more senolytic agent(s) (e.g., DGLA, and/or GLA, and/or D5D inhibitor) is used in an amount and for a time sufficient that selectively kills established senescent cells but is insufficient to kill (destroy, cause the death of) a non-senescent cell in a clinically significant or biologically significant manner. In various embodiments, the DGLA, and/or GLA, and/or D5D inhibitor may selectively kill one or more types of senescent cells (e.g., senescent preadipocytes, senescent endothelial cells, senescent fibroblasts, senescent fibro adipogenic progenitors, senescent skeletal muscle satellite cells, senescent neurons, senescent epithelial cells, senescent mesenchymal cells, senescent smooth muscle cells, senescent macrophages, or senescent chondrocytes).

A senescent cell may exhibit any one or more of the following seven characteristics. (1) Senescence growth arrest is essentially permanent and cannot be reversed by known physiological stimuli. (2) Senescent cells increase in size, sometimes enlarging more than twofold relative to the size of non-senescent counterparts. (3) Senescent cells express a senescence-associated β-galactosidase (SA-β-gal), which partly reflects the increase in lysosomal mass. (4) Many senescent cells express p16INK4a, which is not commonly expressed by quiescent or terminally differentiated cells. (5) Cells that senesce with persistent DNA damage response (DDR) signaling harbor persistent nuclear foci, termed DNA segments with chromatin alterations reinforcing senescence (DNA-SCARS). These foci contain activated DDR proteins and are distinguishable from transient damage foci. DNA-SCARS can include dysfunctional telomeres or telomere dysfunction-induced foci (TIF). (6) Senescent cells express and may secrete molecules associated with senescence, which in certain instances may be observed in the presence of persistent DDR signaling, which in certain instances may be dependent on persistent DDR signaling for their expression. (7) The nuclei of senescent cells lose structural proteins such as Lamin B1 or chromatin-associated proteins such as certain histones and HMGB1 (See, e.g., Freund et al., (2012) Mol. Biol. Cell, 23: 2066-2075; Davalos et al. (2013) J. Cell Biol. 201: 613-629; Ivanov et al. (2013) J. Cell Biol. 202(1): 129-143; Funayama et al., (2006) J. Cell Biol. 175: 869-880; and the like).

Senescent cells and senescent cell associated molecules can be detected by techniques and procedures described in the art. For example, the presence of senescent cells in tissues can be analyzed by histochemistry or immunohistochemistry techniques that detect the senescence marker, SA-beta galactosidase (SA-βgal) (see, e.g., Dimri et al. (1995) Proc. Natl. Acad. Sci. USA, 92: 9363-9367). The presence of the senescent cell-associated polypeptide p16INK4a can be determined by any one of numerous immunochemistry methods practiced in the art, such as immunoblotting analysis. Expression of p16^{INK4a} mRNA in a cell can be measured by a variety of techniques practiced in the art including quantitative PCR. The presence and level of senescent cell associated polypeptides (e.g., polypeptides of the SASP) can be determined by using automated and high throughput assays, such as an automated Luminex array assay described in the art (see, e.g., Coppe et al. (2008) PLoS Biol. 6: 2853-2868).

The presence of senescent cells can also be determined by detection of senescent cell-associated molecules, which include growth factors, proteases, cytokines (e.g., inflammatory cytokines), chemokines, cell-related metabolites, reactive oxygen species (e.g., H₂O₂), and other molecules that stimulate inflammation and/or other biological effects or reactions that may promote or exacerbate the underlying disease of the subject. Senescent cell-associated molecules include those that are described in the art as comprising the senescence-associated secretory phenotype (SASP, i.e., which includes secreted factors which may make up the pro-inflammatory phenotype of a senescent cell), senescent-messaging secretome, and DNA damage secretory program (DDSP). These groupings of senescent cell associated molecules, as described in the art, contain molecules in common and are not intended to describe three separate distinct groupings of molecules. Senescent cell-associated molecules include certain expressed and secreted growth factors, proteases, cytokines, and other factors that may have potent autocrine and paracrine activities (see, e.g., Coppe et al. (2008) PLoS Biol. 6: 2853-2868; Coppe et al. (2006) J. Biol. Chem. 281: 29568-29574; Coppe et al. (2010) PLoS One 5: 39188; Krtolica et al. (2001) Proc. Natl. Acad. Sci. USA, 98: 12072-12077; Parrinello et al. (2005) J. Cell Sci. 118: 485-496; Basisty et al. (2020) PLoS Biol., 18: e3000599). Extracellular matrix (ECM) associated factors include inflammatory proteins and mediators of ECM remodeling and which are strongly induced in senescent cells (see, e.g., Kuilman et al. (2009) Nature Rev. 9: 81-94). Other senescent cell-associated molecules include extracellular polypeptides (proteins) described collectively as the DNA damage secretory program (DDSP) (see, e.g., Sun et al. (2012) Nature Med. 18: 1359-1368). Senescent cell-associated proteins also include cell surface proteins (or receptors) that are expressed on senescent cells, which include proteins that are present at a detectably lower amount or are not present on the cell surface of a non-senescent cell.

Senescence cell-associated molecules include secreted factors that may make up the pro-inflammatory phenotype of a senescent cell (e.g., SASP). These factors include, without limitation, GM-CSF, GROα, GROα,β,γ, IGFBP-7, IL-1α, IL-6, IL-7, IL-8, MCP-1, MCP-2, MIP-1α, MMP-1, MMP-10, MMP-3, Amphiregulin, ENA-78, Eotaxin-3, GCP-2, GITR, HGF, ICAM-1, IGFBP-2, IGFBP-4, IGFBP-5, IGFBP-6, IL-13, IL-1β, MCP-4, MIF, MIP-3α, MMP-12, MMP-13, MMP-14, NAP2, Oncostatin M, osteoprotegerin, PIGF, RANTES, sgp130, TIMP-2, TRAIL-R3, Acrp30, angiogenin, Ax1, bFGF, BLC, BTC, CTACK, EGF-R, Fas, FGF-7, G-CSF, GDNF, HCC-4, 1-309, IFN-γ, IGFBP-1, IGFBP-3, IL-1 R1, IL-11, IL-15, IL-2R-α, IL-6 R, I-TAC, Leptin, LIF, MMP-2, MSP-a, PAI-1, PAI-2, PDGF-BB, SCF, SDF-1, sTNF RI, sTNF RII, Thrombopoietin, TIMP-1, tPA, uPA, uPAR, VEGF, MCP-3, IGF-1, TGF-β3, MIP-1-delta, IL-4, FGF-7, PDGF-BB, IL-16, BMP-4, MDC, MCP-4, IL-10, TIMP-1, Fit-3 Ligand, ICAM-1, Ax1, CNTF, INF-γ, EGF, BMP-6. Additional identified factors, which include those sometimes referred to in the art as senescence messaging secretome (SMS) factors, some of which are included in the listing of SASP polypeptides, include without limitation, IGF1, IGF2, and IGF2R, IGFBP3, IDFBP5, IGFBP7, PA11, TGF-β, WNT2, IL-1α, IL-6, IL-8, and CXCR2-binding chemokines. Cell-associated molecules also include without limitation the factors described in Sun et al., Nature Medicine, supra, and include, including, for example, products of the genes, MMP1, WNT16B, SFRP2, MMP12, SPINK1, MMP10, ENPP5, EREG, BMP6, ANGPTL4, CSGALNACT, CCL26, AREG, ANGPT1, CCK, THBD, CXCL14, NOV, GAL, NPPC, FAM150B, CST1, GDNF, MUCL1, NPTX2, TMEM155, EDNI, PSG9, ADAMTS3, CD24, PPBP, CXCL3, MMP3, CST2, PSG8, PCOLCE2, PSG7, TNFSF15, C17orf67, CALCA, FGF18, IL8, BMP2, MATN3, TFP1, SERPINI 1, TNFFRSF25, and IL23A. Senescent cell-associated proteins also include cell surface proteins (or receptors) that are expressed on senescent cells, which include proteins that are present at a detectably lower amount or are not present on the cell surface of a non-senescent cell.

In certain embodiments, the senolytic agent (DGLA, and/or GLA, and/or D5D inhibitor), alone or in combination with other senolytic agents selectively kill at least senescent fibroblasts and can be useful for treatment of fibrotic diseases. In other embodiments, the senolytic agent DGLA, alone or in combination with other senolytic agents are capable of selectively killing at least senescent endothelial cells, senescent smooth muscle cells, and/or senescent macrophages. Such senolytic agent(s) may be useful for treatment of a cardiovascular disease (e.g., atherosclerosis). In other particular embodiments, the senolytic agent DGLA, alone or in combination with other senolytic agents is capable of selectively killing at least senescent fibroblasts. In still another embodiment, the senolytic agent DGLA, alone or in combination with other senolytic agents, may selectively kill at least senescent brain cells, including neurons and astrocytes. In still another embodiment, the senolytic agent (DGLA, and/or GLA, and/or D5D inhibitor), alone or in combination with other senolytic agents may kill at least senescent retinal pigmented epithelial cells or other senescent epithelial cells (e.g., pulmonary senescent epithelial cells or senescent kidney (renal) epithelial cells). Selective killing of at least senescent pulmonary epithelial cells may be useful for treating pulmonary diseases, such as chronic obstructive pulmonary disease or idiopathic pulmonary fibrosis. In yet other embodiments, the senolytic agent (DGLA, and/or GLA, and/or D5D inhibitor), alone or in combination with other senolytic agents may selectively kill at least senescent immune cells (such as senescent macrophages). In still another embodiment, the senolytic agent (DGLA, and/or GLA, and/or D5D inhibitor), alone or in combination with other senolytic agents may kill at least senescent chondrocytes, which may be useful for treatment of an inflammatory disorder, such as osteoarthritis. In still another embodiment, the senolytic agent (DGLA, and/or GLA, and/or D5D inhibitor), alone or in combination with other senolytic agents may kill senescent fibro adipogenic progenitors or skeletal muscle satellite cells, which may be useful for treatment of skeletal muscle disorders such as sarcopenia or chemotherapy-related fatigue/wasting/physical dysfunction.

Senescent cells that are the targets of the methods described herein may be senescent due to replicative cellular senescence, stress-induced cellular senescence or therapy-induced senescence. A cell that is senescent due to stress may be induced by, but not limited to one or more of, ultraviolet light, reactive oxygen species, chemotherapeutics, environmental toxins, cigarette smoking, ionizing radiation, distortion of chromatin structure, excessive mitogenic signaling, and oncogenic mutations. In a specific embodiment, cellular senescence may be induced by ionizing radiation (IR). A senescent cell that is therapy-induced senescent may have been exposed to DNA-damaging therapy or certain drugs used to treat, for example, HIV-AIDS.

Non-limiting examples of senescent cells may include, but are not limited to, mammary epithelial cells, keratinocytes, cardiac myocytes, chondrocytes, endothelial cells (large vessels), endothelial cells (microvascular), epithelial cells, fibroblasts, follicle dermal papilla cells, hepatocytes, melanocytes, osteoblasts, preadipocytes, primary cells of the immune system, skeletal muscle cells, fibro adipogenic progenitors, skeletal muscle satellite cells, smooth muscle cells, adipocytes, neurons, glial cells, contractile cells, exocrine secretory epithelial cells, extracellular matrix cells, hormone secreting cells, keratinizing epithelial cells, islet cells, lens cells, mesenchymal stem cells, pancreatic acinar cells, paneth cells of the small intestine, primary cells of hemopoietic linage, primary cells of the nervous system, sense organ and peripheral neuron supporting cells, wet stratified barrier epithelial cells and the like.

In certain embodiments, senescent cells that are targets in the methods described herein may be found in renewable tissues, including the vasculature, hematopoietic system, epithelial organs and the stroma. The senescent cells may also be found at sites of aging or chronic age-related pathology, such as osteoarthritis and atherosclerosis. Further, the senescent cell may be associated with benign dysplastic or preneoplastic lesions and benign prostatic hyperplasia. In certain embodiments, the target senescent cell(s) may be found in normal and tumor tissues following DNA-damaging therapy. In a specific embodiment, a senescent cell may be found at a site of aging or age-related pathology.

Use of the Senolytic Agent DGLA in the Treatment of Cancer and/or the Prevention of Therapy Induced Senescent Cells.

### Use of DGLA in the Treatment of Cancer.

Senescence characterized by the accumulation of senescent cells (SASP phenotype) has been implicated in driving neoplastic transformation and tumor aggressiveness associated, inter alia, with the expression/secretion of wide-ranging pro-tumorigenic cytokines, growth factors, and matrix-degrading enzymes. Aging tissues accumulate senescent cells, and the in vivo selective elimination of spontaneously emerging, age-associated senescent cells has been documented to delay tumor formation and deterioration of cardiac, renal, and adipose tissue function. Accordingly, in certain embodiments, the senolytic agent DGLA alone or in combination with other senolytic agents is used to prevent, delay the onset of, slow the progression of and/or treat (e.g., ameliorate one or more symptoms) of cancer.

Non-limiting examples of cancers that may be treated using DGLA, and/or GLA, and/or D5D inhibitor, alone or in combination with other senolytic agents, include, but are not limited to leukemia, a secondary tumor, a solid tumor, acute leukemia, adrenal gland tumor, ameloblastoma, anaplastic carcinoma of the thyroid, angioma, apudoma, argentaffinoma, arrhenoblastoma, ascites tumor, astroblastoma, astrocytoma, ataxia-telangiectasia-associated tumors, basal cell carcinoma, bone cancer, brain tumor, brainstem glioma, breast cancer, Burkitt's lymphoma, cervical cancer, cholangioma, chondroblastoma, chondrosarcoma, chorioblastoma, choriocarcinoma, colon cancer, craniopharyngioma, cystocarcinoma, cystofbroma, cystoma, ductal carcinoma, ductal papilloma, dysgerminoma, encephaloma, endometrial carcinoma, endothelioma, ependymoma, erythroleukemia, Ewing's sarcoma, extra nodal lymphoma, fibro adenoma, fibro sarcoma, follicular cancer of the thyroid, ganglioglioma, gastrinoma cell, glioblastoma multiform, glioma, gonadoblastoma, haemangioblastoma, haemangioendothelioblastoma, haemangioendothelioma, haemangiopericytoma, haematolymphangioma, haemocytoblastoma, haemocytoma, hairy cell leukemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angioforoma, Kaposi sarcoma, kidney tumor, large cell lymphoma, leukemia, lipoma, liver cancer, liver metastases, Lucke carcinoma, lung cancer, lymphadenoma, lymphangioma, lymphocytic leukemia, lymphocytic lymphoma, lymphoedema, lymphoeytoma, lymphoma, malignant mesothelioma, malignant teratoma, mastocytoma, medulloblastome, melanoma, meningioma, mesothelioma, Morton's neuroma, multiple myeloma, myeloid leukemia, myelolipoma, myeloma, myoblastoma, myxoma, nasopharyngeal carcinoma, neuroblastoma, neurofibroma, neuroglioma, neuroma, non-Hodgkin's lymphoma, oligodendroglioma, optic glioma, osteochondroma, osteogenic sarcoma, osteosarcoma, ovarian cancer, pancoast tumor, pancreatic cancer, phaeochromocytoma, plasmacytoma, primary brain tumor, progonoma, prolactinoma, renal cell carcinoma, retinoblastoma, rhabdosarcoma, sarcoma, skin cancer, small cell carcinoma, squamous cell carcinoma, T-cell lymphoma, testicular cancer, thymoma, trophoblastic tumor, Wilm's tumor, and the like.

In certain embodiments the senolytic agent (DGLA, and/or GLA, and/or D5D inhibitor), alone or in combination with other senolytic agents can be used in conjunction with other treatments for cancer that may induce senescence, such as irradiation or chemotherapy (for example, treatment with doxorubicin, palbociclib, ribociclib or abemaciclib, or other chemotherapeutic agents). Thus, by treating at the same time, or after the other treatment, the agent can:
1) Eliminate cancer cells that have been pushed to senescence; and/or
2) Eliminate or reduce certain side effects produced by senescent cells

such as inflammation, promotion of cancer growth, promotion of metastasis and other side effects of chemotherapy or radiotherapy; and/or
3) Reduce or eliminate precancerous lesions; and/or
4) Eliminate or reduce cells that undergo senescence by treatment with CDK4/CDK6 inhibitors such as Palbociclib and genotoxic or cytotoxic drugs, which induce senescence.

In certain embodiments the senolytic agent (DGLA, and/or GLA, and/or D5D inhibitor), alone or in combination with other senolytic agents can reduce or eliminate precancerous (or pre-neoplastic) lesions. Senescent cells often exist in premalignant tumors, but not in malignant ones, although they can exist in stroma surrounding malignant tumors. In this regard, it is understood that a substantial number of cells in premalignant tumors undergo oncogene-induced senescence, but that cells in malignant tumors are unable to do this owing to the loss of oncogene-induced senescence effectors such as p16^{INK4a} or p53 (Collado et al. (2005) Nature, 436: 642). Thus, in certain embodiments the use of DGLA, alone or in combination with other senolytic agents for reducing or eliminating precancerous (or pre-neoplastic) lesions is contemplated.

In certain embodiments the effective agent(s) (DGLA, and/or GLA, and/or D5D inhibitor), alone or in combination with other senolytic agents can eliminate cancer cells that have been pushed to senescence. In one preferred embodiment, the agent delays tumorigenesis.

In certain embodiments DGLA, alone or in combination with other senolytic agents can eliminate or reduce effects produced by senescent cells that drive cancer occurrence and/or progression and/or metastasis formation. In this regard, it is noted that although cellular senescence suppresses tumorigenesis early in life, studies have shown that it may promote cancer in aged organisms (see, e.g., Krtolica et al. (2001) Proc. Natl. Acad. Sci. USA, 98(21): 12072-12077). Oncogene-induced senescence is classically considered a tumor defense barrier. However, several studies have shown that under certain circumstances, senescent cells may promote tumor progression because of their secretory phenotype (see, e.g., Angelini et al. (2013) Cancer Res. 73(1): 450-458).

In a particular embodiment, methods are provided for treating or preventing (i.e., reducing the likelihood of occurrence or development of) a senescence cell associated disease (or disorder or condition), which is metastasis. The active agent(s) described herein (DGLA, and/or GLA, and/or D5D inhibitor), alone or in combination with other senolytic agents may also be used according to the methods described herein for treating or preventing (i.e., reducing the likelihood of occurrence of) metastasis (i.e., the spreading and dissemination of cancer or tumor cells) from one organ or tissue to another organ or tissue in the body.

Accordingly, DGLA, and/or GLA, and/or a D5D inhibitor, alone or in combination with other senolytic agents when administered to a subject who has a cancer according to the methods described herein may inhibit tumor proliferation. Metastasis of a cancer occurs when the cancer cells (e.g., tumor cells) spread beyond the anatomical site of origin and initial colonization to other areas throughout the body of the subject. Tumor size may be determined by tumor size, which can be measured in various ways familiar to a person skilled in the art, such as by PET scanning, MRI, CAT scan, biopsy, for example. The effect of the therapeutic agent on tumor proliferation may also be evaluated by examining differentiation of the tumor cells.

As used herein and in the art, the terms cancer or tumor are clinically descriptive terms that encompass diseases typically characterized by cells exhibiting abnormal cellular proliferation. The term cancer is generally used to describe a malignant tumor or the disease state arising from the tumor. Alternatively, an abnormal growth may be referred to in the art as a neoplasm. The term tumor, such as in reference to a tissue, generally refers to any abnormal tissue growth that is characterized, at least in part, by excessive and abnormal cellular proliferation. A tumor may be metastatic and capable of spreading beyond its anatomical site of origin and initial colonization to other areas throughout the body of the subject. A cancer may comprise a solid tumor or may comprise a “liquid” tumor (e.g., leukemia and other blood cancers).

Cells are induced to senesce by cancer therapies, such as radiation and certain chemotherapy drugs. The presence of senescent cells increases the presence of inflammatory molecules by virtue of the SASP (see description herein of senescent cells), that can promote tumor progression, which may include promoting tumor growth and increasing tumor size, promoting metastasis, and altering differentiation. When senescent cells are destroyed, tumor progression is significantly inhibited, resulting in tumors of small size and with little or no observed metastatic growth (see, e.g., PTC Patent Publication No. WO 2013/090645).

In one embodiment, methods are provided for preventing (i.e., reducing the likelihood of occurrence of), inhibiting, or retarding metastasis in a subject who has a cancer by administering DGLA, and/or GLA, and/or a D5D inhibitor, alone or in combination with other senolytic agents. In a particular embodiment, DGLA, and/or GLA, and/or a D5D inhibitor, alone or in combination with other senolytic agents is administered on one or more days within a treatment window (i.e., treatment course) of no longer than 7 days or 14 days. In other embodiments, the treatment course is no longer than 2, 3, 4, 5, 6, 7, 8, 9, 10, 11, 12, 13, 14, 15, 16, 17, 18, 19, 20, or no longer than 21 days. In other embodiments, the treatment course is a single day. In certain embodiments, the DGLA, and/or GLA, and/or D5D inhibitor, alone or in combination with other senolytic agents is administered on two or more days within a treatment window of no longer than 7 days or 14 days, on 3 or more days within a treatment window of no longer than 7 days or 14 days; on 4 or more days within a treatment window of no longer than 7 days or 14 days; on 5 or more days within a treatment window of no longer than 7 days or 14 days; on 6, 7, 8, 9, 10, 11, 12, 13, or 14 days within a treatment window of no longer than 7 days or 14 days. In certain embodiments, when the DGLA, and/or GLA, and/or D5D inhibitor, alone or in combination with other senolytic agents is administered to a subject for a treatment window of 3 days or more, the agent may be administered every 2nd day (i.e., every other day). In other certain embodiments, when the DGLA, and/or GLA, and/or D5D inhibitor, alone or in combination with other senolytic agents is administered to a subject for a treatment window of 4 days or more, the agent may be administered every 3rd day (i.e., every other third day).

### Use of DGLA in the Prevention or Treatment of Therapy Induced Senescent Cells

Senescence is induced by a range of cancer therapies, including radiation, chemotherapies, and several targeted therapies. In certain cancer types, this therapy-induced senescence (TIS) promotes invasive and metastatic phenotypes. Eliminating TIS cells has been reported to reduce many side effects of cancer drugs, including bone marrow suppression, cardiac dysfunction, fatigue, and also to reduce cancer recurrence.

Accordingly in certain illustrative, but non-limiting, embodiments, DGLA, and/or GLA, and/or D5D inhibitor alone, or in combination with one or more additional senolytic agents, can be used in the treatment of chronic or long-term chemotherapy-induced or radiotherapy-induced side effects. Certain toxic effects can appear long after treatment and can result from damage to an organ or system by the therapy. Organ dysfunction, for example, neurological, pulmonary, cardiovascular, and endocrine dysfunction, can be observed in subjects who were treated for cancers during childhood. Chronic or late toxic side effects that occur in subjects who received chemotherapy or radiation therapy include, but are not limited to, cardiomyopathy, congestive heart disease, inflammation, early menopause, osteoporosis, infertility, impaired cognitive function, peripheral neuropathy, secondary cancers, cataracts and other vision problems, hearing loss, chronic fatigue, reduced lung capacity, and lung disease.

Accordingly, in various embodiments, methods are also provided for killing therapy-induced senescent cells. The methods comprise administering a composition comprising a therapeutically effective amount of the senolytic agent DGLA, and/or GLA, and/or D5D inhibitor to a subject that has received DNA-damaging therapy or prophylactically to a subject that is about to undergo a DNA-damaging therapy, or concurrently with a DNA damaging therapy and killing therapy induced-senescent cells in normal and/or tumor tissues following DNA-damaging therapy.

Based on the observation that ionizing radiation and various chemotherapeutic agents elicit a marked senescence response in vivo, therapy-induced senescent cells may be a cause of long-term ramifications after DNA-damaging therapy, such as cancer therapy. As such, the systemic accumulation of therapy-induced senescent cells may drive accelerated physical decline in cancer survivors. Accelerated physical decline may also be referred to as accelerated aging. Accordingly, once neoplastic cells are removed or eliminated by systemic radiation or chemotherapy, senescence may be triggered in a variety of other organs, leading to long-term ramifications for the patient. Long-term ramifications may include reduced quality of life predisposing the subject to disabilities and comorbidities. For example, a subject that has received DNA-damaging therapy may experience a disproportionate decline in physical function, such as inability to climb stairs, or to reach up to put things onto shelves and/or increased functional disabilities such as difficulty eating, dressing and maintaining adequate hygiene. Additionally, late effects of ionizing radiation may include long-term bone marrow injury and/or lung fibrosis. Long-term bone marrow injury can promote hypoplastic anemia and/or myelodysplastic syndrome or leukemia. Additionally, it has been demonstrated that following ionizing radiation, senescent cells in lung, muscle and brain are greatly increased. These long-term ramifications provide a link between accelerated aging and cancer treatment. Accordingly, in various embodiments, administration of DGLA, and/or GLA, and/or a D5D inhibitor alone or along with one or more other senolytic agents is contemplated, e.g., as an adjuvant (adjunct) therapy in the treatment of a cancer.

Illustrative cancers in which administration of DGLA, and/or GLA, and/or a D5D inhibitor alone or in combination with one or more other senolytic agents may provide an appropriate adjuvant therapy include, but are not limited to, a cancer selected from the group consisting of leukemia, a secondary tumor, a solid tumor, acute leukemia, adrenal gland tumor, ameloblastoma, anaplastic carcinoma of the thyroid, angioma, apudoma, argentaffinoma, arrhenoblastoma, ascites tumor, astroblastoma, astrocytoma, ataxia-telangiectasia-associated tumors, basal cell carcinoma, bone cancer, brain tumor, brainstem glioma, breast cancer, Burkitt's lymphoma, cervical cancer, cholangioma, chondroblastoma, chondrosarcoma, chorioblastoma, choriocarcinoma, colon cancer, craniopharyngioma, cystocarcinoma, cystofbroma, cystoma, ductal carcinoma, ductal papilloma, dysgerminoma, encephaloma, endometrial carcinoma, endothelioma, ependymoma, erythroleukemia, Ewing's sarcoma, extra nodal lymphoma, fibro adenoma, fibro sarcoma, follicular cancer of the thyroid, ganglioglioma, gastrinoma cell, glioblastoma multiform, glioma, gonadoblastoma, haemangioblastoma, haemangioendothelioblastoma, haemangioendothelioma, haemangiopericytoma, haematolymphangioma, haemocytoblastoma, haemocytoma, hairy cell leukemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angioforoma, Kaposi sarcoma, kidney tumor, large cell lymphoma, leukemia, lipoma, liver cancer, liver metastases, Lucke carcinoma, lung cancer, lymphadenoma, lymphangioma, lymphocytic leukemia, lymphocytic lymphoma, lymphoedema, lymphoeytoma, lymphoma, malignant mesothelioma, malignant teratoma, mastocytoma, medulloblastome, melanoma, meningioma, mesothelioma, Morton's neuroma, multiple myeloma, myeloid leukemia, myelolipoma, myeloma, myoblastoma, myxoma, nasopharyngeal carcinoma, neuroblastoma, neurofibroma, neuroglioma, neuroma, non-Hodgkin's lymphoma, oligodendroglioma, optic glioma, osteochondroma, osteogenic sarcoma, osteosarcoma, ovarian cancer, pancoast tumor, pancreatic cancer, phaeochromocytoma, plasmacytoma, primary brain tumor, progonoma, prolactinoma, renal cell carcinoma, retinoblastoma, rhabdosarcoma, sarcoma, skin cancer, small cell carcinoma, squamous cell carcinoma, T-cell lymphoma, testicular cancer, thymoma, trophoblastic tumor, Wilm's tumor, and the like.

Non-limiting examples of DNA-damaging therapy and/or cytotoxic therapy may include gamma-irradiation, alkylating agents such as nitrogen mustards (chlorambucil, cyclophosphamide, ifosfamide, melphalan), nitrosoureas (streptozocin, carmustine, lomustine), alkyl sulfonates (busulfan), triazines (dacarbazine, temozolomide) and ethylenimines (thiotepa, altretamine), platinum drugs such as cisplatin, carboplatin, oxalaplatin, antimetabolites such as 5-fluorouracil, 6-mercaptopurine, capecitabine, cladribine, clofarabine, cytarabine, floxuridine, fludarabine, gemcitabine, hydroxyurea, methotrexate, pemetrexed, pentostatin, thioguanine, anthracyclines such as daunorubicin, doxorubicin, epirubicin, idarubicin, anti-tumor antibiotics such as actinomycin-D, bleomycin, mitomycin-C, topoisomerase inhibitors such as topoisomerase I inhibitors (topotecan, irinotecan) and topoisomerase II inhibitors (etoposide, teniposide, mitoxantrone), mitotic inhibitors such as taxanes (paclitaxel, docetaxel), epothilones (ixabepilone), vinca alkaloids (vinblastine, vincristine, vinorelbine), estramustine, cyclin-dependent kinase inhibitors (roscovitine, palbociclib, abemaciclib, olaparib), epigenetic modifiers (curcumin, valproic acid), and HIV medications such as NRTIs (Nucleoside Reverse Transcriptase Inhibitors), NNRTIs (Non-Nucleoside Reverse Transcriptase Inhibitors), and protease inhibitors (azidothymidine, tenofovir, emtricitabine, abacavir, nevirapine, atazanavir, lopinavir). In various embodiments, administration of the active agent(s) described herein (DGLA, and/or GLA, and/or D5D inhibitor) alone or in combination with one or more other senolytic agents is contemplated, e.g., as an adjuvant (adjunct) to a therapeutic regimen comprising administration of one or more of the above-identified DNA-damaging therapeutics.

It will be recognized that the senolytic agent DGLA, and/or GLA, and/or a D5D inhibitor, alone or in combination with other senolytic agents, can eliminate or reduce chemotherapy-induced (or radiation-induced) senescence, for example, during or after treatment. Thus, in one illustrative embodiment, the senolytic agent(s) can be used in combination treatment with a chemotherapeutic agent, where the agent is administered separately, sequentially or concomitantly with the chemotherapeutic agent. Additionally, or alternatively, in certain embodiments, the senolytic agent(s) can be used in combination treatment with radiation treatments, where the senolytic agent is administered separately, sequentially or concomitantly with the radiation treatment(s).

In certain embodiments the senolytic agent (DGLA, and/or GLA, and/or a D5D inhibitor), alone or in combination with other senolytic agents, can eliminate or reduce senescence induced by treatment with a CDK inhibitor, for example, a CDK4 or CDK6 inhibitor such as Palbociclib. Thus, in one illustrative embodiment, the agent can be used in combination treatment with a CDK4 or CDK6 inhibitor, where the agent is administered separately, sequentially or concomitantly with the CDK4 or CDK6 inhibitor.

In another illustrative embodiment, the senolytic agent (DGLA, and/or GLA, and/or a D5D inhibitor), alone or in combination with other senolytic agents can eliminate or reduce Palbociclib-induced senescence. In the context of eliminating or reducing Palbociclib-induced senescence, administration of the senolytic agent(s) can potentially prevent cancer remission as cells reenter the cell cycle (see, e.g., Cadoo et al. (2014) Breast Cancer: Targets and Therapy, 6: 123-133).

In certain embodiments, e.g., when chemotherapy or radiotherapy is administered in a treatment cycle of at least one day on-therapy (e.g., chemotherapy or radiotherapy)) followed by at least 3, 4, 5, 6, 7, 8, 9, 10, 11, 12, 13, 14 (or about 2 weeks), 15, 16, 17, 18, 19, 20, 21 (or about 3 weeks) days, or about 4 weeks (about one month) off-therapy (e.g., off chemo- or radio-therapy), DGLA alone, or in combination with one or more additional senolytic agents can be administered on one or more days during the off-therapy time interval (time period) beginning on or after the second day of the off-therapy time interval and ending on or before the last day of the off-therapy time interval. By way of illustrative example, if n is the number of days off-therapy, then the active agent(s) described herein (DGLA, and/or GLA, and/or a D5D inhibitor) alone, or in combination with one or more additional senolytic agents are administered on at least one day and no more than one day prior of the off-therapy time interval. In a certain particular embodiment, when chemotherapy or radiotherapy is administered in a treatment cycle of at least one day on-therapy (e.g., chemotherapy or radiotherapy) followed by at least one week off-therapy, DGLA, and/or GLA, and/or a D5D inhibitor alone, or in combination with one or more additional senolytic agents are administered on one or more days during the off-therapy time interval beginning on or after the second day of the off-therapy time interval and ending on or before the last day of the off-therapy time interval. In a more specific illustrative embodiment, when chemotherapy or radiotherapy is administered in a treatment cycle of at least one day on-therapy (e.g., chemotherapy or radiotherapy) followed by at least one week off-therapy, DGLA, and/or GLA, and/or a D5D inhibitor alone, or in combination with one or more additional senolytic agents, are administered on one day that is the sixth day of the off-therapy time interval. In other specific embodiments, when chemotherapy or radiotherapy is administered in a treatment cycle of at least one day on-therapy (e.g., chemotherapy or radiotherapy) followed by at least two weeks off-therapy, DGLA, and/or GLA, and/or a D5D inhibitor alone, or in combination with one or more additional senolytic agents are administered beginning on the sixth day of the off-chemo- or radio-therapy time interval and ending at least one day or at least two days prior to the first day of a subsequent chemotherapy or radiation therapy treatment course. By way of example, if the off-chemo- or radio-therapy time interval is two weeks, DGLA, and/or GLA, and/or a D5D inhibitor alone, or in combination with one or more additional senolytic agents may be administered on at least one and on no more than 7 days (e.g., 1, 2, 3, 4, 5, 6, or 7 days) of the off-therapy time interval beginning on the sixth day after the chemotherapy or radiotherapy course ends (e.g., the sixth day of the off chemo-radio-therapy interval). When the off-chemo- or radio-therapy time interval is at least three weeks, DGLA, and/or GLA, and/or a D5D inhibitor alone, or in combination with one or more additional senolytic agents may be administered on at least one day and on no more than 14 days (e.g., 1-14 days: 1, 2, 3, 4, 5, 6, 7, 8, 9, 10, 11, 12, 13, or 14 days) of the off-therapy time interval beginning on the sixth day after the chemotherapy or radiotherapy course ends. In other embodiments, depending on the off-chemo-radio-therapy interval, the senolytic agent treatment course is at least one day and no longer than 2, 3, 4, 5, 6, 7, 8, 9, 10, 11, 12, 13, 14, 15, 16, 17, 18, 19, 20, or no more than 21 days (e.g., 1-21 days), provided that administration of DGLA, and/or GLA, and/or a D5D inhibitor alone, or in combination with one or more additional senolytic agents are is not concurrent with the chemotherapy or radiotherapy. In certain embodiments, the senolytic agent treatment course is a single day. In certain embodiments, DGLA, and/or GLA, and/or a D5D inhibitor alone, or in combination with one or more additional senolytic agents are administered on two or more days within a treatment window of no longer than 14 days, on 3 or more days within a treatment window of no longer than 14 days; on 4 or more days within a treatment window of no longer than 14 days; on 5 or more days within a treatment window of no longer than 14 days; on 6, 7, 8, 9, 10, 11, 12, 13, or 14 days within treatment window of no longer than 14 days. In certain embodiments, when the at least one senolytic agent (e.g., DGLA, and/or GLA, and/or a D5D inhibitor) is administered to a subject during a treatment course of 3 days or more, the agent may be administered every 2^{nd} day (e.g., every other day). In other certain embodiments, when the at least one senolytic agent (e.g., DGLA) is administered to a subject during a treatment course of 4 days or more, the agent may be administered every 3^{rd} day (e.g., every other third day).

Many chemotherapy and radiotherapy treatment regimens comprise a finite number of cycles of on-drug therapy followed by off-drug therapy or comprise a finite timeframe in which the chemotherapy or radiotherapy is administered. Such cancer treatment regimens may also be called treatment protocols. The protocols are typically determined by clinical trials, and clinical staff in conjunction with the subject to be treated. The number of cycles of a chemotherapy or radiotherapy or the total length of time of a chemotherapy or radiotherapy regimen can vary depending on the patient's response to the cancer therapy. The timeframe for such treatment regimens is readily determined by a person skilled in the oncology art. In another embodiment, for treating metastasis, DGLA, and/or GLA, and/or a D5D inhibitor alone, or in combination with one or more additional senolytic agents may be administered after the treatment regimen of chemotherapy or radiotherapy has been completed. In one particular illustrative embodiment, DGLA, and/or GLA, and/or a D5D inhibitor alone, or in combination with one or more additional senolytic agents are administered after the chemotherapy or radiotherapy has been completed on one or more days within the treatment window (e.g., senolytic agent treatment course) of no longer than 14 days. In other embodiments, the senolytic agent treatment course is no longer than 2, 3, 4, 5, 6, 7, 8, 9, 10, 11, 12, 13, 14, 15, 16, 17, 18, 19, 20, or no more than 21 days. In other embodiments, the treatment course is a single day. In certain embodiments, DGLA, and/or GLA, and/or a D5D inhibitor alone, or in combination with one or more additional senolytic agents are administered on two or more days within a treatment window of no longer than 14 days, on 3 or more days within a treatment window of no longer than 14 days; on 4 or more days within a treatment window of no longer than 14 days; on 5 or more days within a treatment window of no longer than 14 days; on 6, 7, 8, 9, 10, 11, 12, 13, or 14 days within treatment window of no longer than 14 days. In certain embodiments, when DGLA alone, or in combination with one or more additional senolytic agents are administered to a subject after chemotherapy or radiotherapy for a treatment window of 3 days or more, the agent(s) may be administered every 2^{nd} day (e.g., every other day). In other certain embodiments, when DGLA, and/or GLA, and/or a D5D inhibitor alone, or in combination with one or more additional senolytic agents are administered to a subject for a treatment window of 4 days or more, the senolytic agents may be administered every 3rd day (e.g., every other third day). In one embodiment, the treatment with the senolytic agent(s) (e.g., DGLA, and/or GLA, and/or a D5D inhibitor) may be initiated at least 1, 2, 3, 4, 5, 6, 7, 8, 9, 10, 11, 12, 13, or 14 days or later after the cancer treatment regimen has been completed. In a more particular illustrative embodiment, the treatment with DGLA alone, or in combination with one or more additional senolytic agents, may be initiated at least 6, 7, 8, 9, 10, 11, 12, 13, or 14 days or later after the cancer treatment regimen has been completed.

Delaying the Onset or Progression and/or Treatment of Age-Related Diseases

It has been demonstrated that senescent cells drive age-related pathologies, and that selective elimination of these cells can prevent or delay age-related deterioration. Thus, senescent cells may be therapeutic targets in the treatment of aging and age-related disease. As such, removal of senescent cells may delay tissue dysfunction and extend health span. Clearance of senescent cells is expected to improve the tissue milieu, thereby improving the function of the remaining non-senescent cells.

Accordingly, in various embodiments, methods are provided for delaying at least one feature of aging in a subject and/or for ameliorating one or more symptoms of aging in a subject, where the method involves administering a therapeutically effective amount of DGLA, and/or GLA, and/or a D5D inhibitor alone, or in combination with one or more additional senolytic agents to a subject. As used herein, “a feature of aging” may include, but is not limited to, systemic decline of the immune system, muscle atrophy and decreased muscle strength, decreased skin elasticity, delayed wound healing, retinal atrophy, reduced lens transparency, reduced hearing, osteoporosis, sarcopenia, hair graying, skin wrinkling, poor vision, frailty, and cognitive impairment.

An age-related pathology may include any disease or condition that is fully or partially mediated by the induction or maintenance of a non-proliferating or senescent state in a cell or a population of cells in a subject. Non-limiting examples include age-related tissue or organ decline which may lack visible indication of pathology, or overt pathology such as a degenerative disease or a function-decreasing disorder. For example, Alzheimer's disease, Parkinson's disease, cataracts, macular degeneration, glaucoma, atherosclerosis, acute coronary syndrome, myocardial infarction, stroke, hypertension, idiopathic pulmonary fibrosis (IPF), chronic obstructive pulmonary disease (COPD), osteoarthritis, sarcopenia, type 2 diabetes, obesity, coronary artery disease, cerebrovascular disease, periodontal disease, and cancer treatment-related disability such as atrophy and fibrosis in various tissues, brain and heart injury, and therapy-related myelodysplastic syndromes. Additionally, an age-related pathology may include an accelerated aging disease such as progeroid syndromes (i.e. Hutchinson-Gilford progeria syndrome, Werner syndrome, Bloom syndrome, Rothmund-Thomson Syndrome, Cockayne syndrome, trichothiodystrophy, combined xeroderma pigmentosum-Cockayne syndrome, restrictive dermopathy), ataxia telangiectasia, Fanconi anemia, Friedreich's ataxia, dyskeratosis congenital, aplastic anemia, IPF, and others. A method of identifying an age-related disease or condition as described herein may include detecting the presence of senescent cells.

Age related diseases or conditions can also include, for example, renal dysfunction, kyphosis, herniated intervertebral disc, frailty, hair loss, hearing loss, vision loss (blindness or impaired vision), muscle fatigue, skin conditions, skin nevi, diabetes, metabolic syndrome, and sarcopenia. Vision loss refers to the absence of vision when a subject previously had vision. Various scales have been developed to describe the extent of vision and vision loss based on visual acuity. Age-related diseases and conditions also include dermatological conditions, for example without limitation, treating one or more of the following conditions: wrinkles, including superficial fine wrinkles; hyperpigmentation; scars; keloids; dermatitis; psoriasis; eczema (including seborrheic eczema); rosacea; vitiligo; ichthyosis vulgaris; dermatomyositis; and actinic keratosis.

Frailty has been defined as a clinically recognizable state of increased vulnerability resulting from aging-associated decline in reserve and function across multiple physiologic systems that compromise a subject's ability to cope with every day or acute stressors. Frailty may be characterized by compromised energetics characteristics such as low grip strength, low energy, slowed waking speed, low physical activity, and/or unintentional weight loss. Studies have suggested that a patient may be diagnosed with frailty when three of five of the foregoing characteristics are observed (see, e.g., Fried et al. (2001) J. Gerontol. A Biol. Sci. Med, Sci. 56(3): M146-M156; Xue (2011) Clin. Geriatr. Med. 27(1): 1-15). In certain embodiments, aging and diseases and disorders related to aging may be treated or prevented (i.e., the likelihood of occurrence of is reduced) by administering DGLA, and/or GLA, and/or a D5D inhibitor alone, or in combination with one or more additional senolytic agents. The DGLA, and/or GLA, and/or a D5D inhibitor alone, or in combination with one or more additional senolytic agents, may inhibit senescence of adult stem cells or inhibit accumulation, kill, or facilitate removal of adult stem cells that have become senescent (see, e.g., Park et al. (2004) J. Clin. Invest. 113: 175-179, and Sousa-Victor (2014) Nature, 506: 316-321 describing importance of preventing senescence in stem cells to maintain regenerative capacity of tissues).

The effectiveness of DGLA, and/or GLA, and/or a D5D inhibitor alone, or in combination with one or more additional senolytic agents, with respect to treating a senescence-associated disease or disorder described herein can readily be determined by a person skilled in the medical and clinical arts. One or any combination of diagnostic methods appropriate for the particular disease or disorder, which methods are well known to a person skilled in the art, including physical examination, patient self-assessment, assessment and monitoring of clinical symptoms, performance of analytical tests and methods, including clinical laboratory tests, physical tests and exploratory surgery, for example, may be used for monitoring the health status of the subject and the effectiveness of the senolytic agent(s) (DGLA, and/or GLA, and/or a D5D inhibitor). The effects of the methods of treatment described herein can be analyzed using techniques known in the art, such as comparing symptoms of patients suffering from or at risk of a particular disease or disorder that have received the pharmaceutical composition comprising DGLA alone, or in combination with one or more additional senolytic agents, with those of patients who were not treated with the senolytic agent or who received a placebo treatment.

Therapeutic benefit for subjects to whom DGLA, and/or GLA, and/or a D5D inhibitor alone, or in combination with one or more additional senolytic agents described herein are administered, includes, for example, an improved clinical outcome, wherein the object is to prevent or slow or retard (lessen) an undesired physiological change associated with the disease, or to prevent or slow or retard (lessen) the expansion or severity of such disease. As discussed herein, effectiveness of the DGLA, and/or GLA, and/or a D5D inhibitor alone, or in combination with one or more additional senolytic agents, may include beneficial or desired clinical results that comprise, but are not limited to, abatement, lessening, or alleviation of symptoms that result from or are associated with the disease to be treated; decreased occurrence of symptoms; improved quality of life; longer disease-free status (i.e., decreasing the likelihood or the propensity that a subject will present symptoms on the basis of which a diagnosis of a disease is made); diminishment of extent of disease; stabilized (i.e., not worsening) state of disease; delay or slowing of disease progression; amelioration or palliation of the disease state; and remission (whether partial or total), whether detectable or undetectable; and/or overall survival. In certain embodiments, the effectiveness of DGLA, and/or GLA, and/or a D5D inhibitor alone, or in combination with one or more additional senolytic agents described herein may also mean prolonging survival when compared to expected survival if a subject were not receiving the senolytic agent(s).

In certain embodiments, administration of DGLA, and/or GLA, and/or a D5D inhibitor alone, or in combination with one or more additional senolytic agents described herein can prolong survival when compared to expected survival if a subject were not receiving treatment. Subjects in need of treatment include those who already have the disease or disorder as well as subjects prone to have or at risk of developing the disease or disorder, and those in which the disease, condition, or disorder is to be treated prophylactically. A subject may have a genetic predisposition for developing a disease or disorder that would benefit from clearance of senescent cells or may be of a certain age wherein receiving DGLA alone, or in combination with one or more additional senolytic agents would provide clinical benefit to delay development or reduce severity of a disease, including an age-related disease or disorder.

In another embodiment, a method is provided for treating a senescence-associated disease or disorder that further comprises identifying a subject who would benefit from treatment with a DGLA alone, or in combination with one or more additional senolytic agents described herein (i.e., phenotyping; individualized treatment). This method comprises first detecting the level of senescent cells in the subject, such as in a particular organ or tissue of the subject. A biological sample may be obtained from the subject, for example, a blood sample, serum or plasma sample, biopsy specimen, body fluids (e.g., lung lavage, ascites, mucosal washings, synovial fluid, vitreous fluid, spinal fluid, urine), bone marrow, lymph nodes, tissue explant, organ culture, or any other tissue or cell preparation from a subject. The level of senescent cells may be determined according to any of the in vitro assays or techniques described herein. For example, senescence cells may be detected by morphology (as viewed by microscopy, for example); production of senescence associated markers such as, senescence-associated β-galactosidase (SA-β-gal), p16INK4a, p21, PAI-1, or any one or more SASP factors (e.g., IL-6, MMP3). The senescent cells and non-senescent cells of the biological sample may also be used in an in vitro cell culture assay in which the cells are exposed to DGLA, and/or GLA, and/or a D5D inhibitor alone, or in combination with one or more additional senolytic agents described herein to determine the capability of the DGLA alone, or in combination with one or more additional senolytic agents, to kill the subject's senescent cells without undesired toxicity to non-senescent cells. As positive controls in these assays, the assay may incorporate the DGLA, and/or GLA, and/or a D5D inhibitor alone, or in combination with one or more additional senolytic agents described herein. In addition, these methods may be used to monitor the level of senescent cells in the subject before, during, and after treatment with DGLA, and/or GLA, and/or a D5D inhibitor alone, or in combination with one or more additional senolytic agents. In certain embodiments, the presence of senescence cells, may be detected (e.g., by determining the level of a senescent cell marker mRNA, for example), and the treatment course and/or non-treatment interval can be adjusted accordingly.

As indicated above, methods are provided herein for treating conditions, diseases, or disorders related to, associated with, or caused by cellular senescence, including age-related diseases and disorders in a subject in need thereof. A senescence-associated disease or disorder may also be called herein a senescence-associated disease. Senescence-associated diseases and disorders include, for example, cardiovascular diseases and disorders, inflammatory diseases and disorders, autoimmune diseases and disorders, pulmonary diseases and disorders, eye diseases and disorders, metabolic diseases and disorders, neurological diseases and disorders (e.g., neurodegenerative diseases and disorders); age-related diseases and disorders induced by senescence; skin conditions; age-related diseases; dermatological diseases and disorders; and transplant related diseases and disorders. A prominent feature of aging is a gradual loss of function or degeneration that occurs at the molecular, cellular, tissue, or organismal levels. Age-related degeneration gives rise to well-recognized pathologies, such as sarcopenia, atherosclerosis and heart failure, osteoporosis, pulmonary insufficiency, renal failure, neurodegeneration (including macular degeneration, Alzheimer's disease, and Parkinson's disease), and many others. Although different mammalian species vary in their susceptibilities to specific age-related pathologies, collectively, age-related pathologies generally rise with approximately exponential kinetics beginning at about the mid-point of the species-specific life span (e.g., 50-60 years of age for humans) (see, e.g., Campisi (2013) Annu. Rev. Physiol. 75: 685-705; Naylor et al. (2013) Clin. Pharmacol. Ther. 93:105-16).

Examples of senescence-associated conditions, disorders, or diseases that may be treated by administering DGLA, and/or GLA, and/or a D5D inhibitor alone, or in combination with one or more additional senolytic agents according to the methods described herein, include cognitive diseases (e.g., mild cognitive impairment (MCI), Alzheimer's disease and other dementias; Huntington's disease); cardiovascular disease (e.g., atherosclerosis, cardiac diastolic dysfunction, aortic aneurysm, angina, arrhythmia, cardiomyopathy, congestive heart failure, coronary artery disease, myocardial infarction, endocarditis, hypertension, carotid artery disease, peripheral vascular diseases, cardiac stress resistance, cardiac fibrosis); metabolic diseases and disorders (e.g., obesity, diabetes, metabolic syndrome); motor function diseases and disorders (e.g., Parkinson's disease, motor neuron dysfunction (MND); Huntington's disease); cerebrovascular disease; emphysema; osteoarthritis; benign prostatic hypertrophy; pulmonary diseases (e.g., idiopathic pulmonary fibrosis, chronic obstructive pulmonary disease (COPD), emphysema, obstructive bronchiolitis, asthma); inflammatory/immune diseases and disorders (e.g., osteoarthritis, eczema, psoriasis, osteoporosis, mucositis, transplantation related diseases and disorders); ophthalmic diseases or disorders (e.g., age-related macular degeneration, cataracts, glaucoma, vision loss, presbyopia); diabetic ulcer; metastasis; a chemotherapeutic side effect, a radiotherapy side effect; aging-related diseases and disorders (e.g., kyphosis, renal dysfunction, frailty, hair loss, hearing loss, muscle fatigue, skin conditions, sarcopenia, and herniated intervertebral disc) and other age-related diseases that are induced by senescence (e.g., diseases/disorders resulting from irradiation, chemotherapy, smoking tobacco, eating a high fat/high sugar diet, and environmental factors); wound healing; skin nevi; fibrotic diseases and disorders (e.g., cystic fibrosis, renal fibrosis, liver fibrosis, pulmonary fibrosis, oral submucous fibrosis, cardiac fibrosis, and pancreatic fibrosis). In certain embodiments, any one or more of the diseases or disorders described above or herein may be excluded.

In a more specific embodiment, methods are provided for treating a senescence-associated disease or disorder by killing senescent cells (i.e., established senescent cells) associated with the disease or disorder in a subject who has the disease or disorder by administering DGLA, and/or GLA, and/or a D5D inhibitor alone or in combination with one or more additional senolytic agents, wherein the disease or disorder is osteoarthritis; idiopathic pulmonary fibrosis; chronic obstructive pulmonary disease (COPD); or atherosclerosis.

In certain embodiments, subjects (e.g., patients, individuals (human or non-human animals)) who may benefit from use of DGLA, and/or GLA, and/or a D5D inhibitor as for the treatment of a pathology associated with aging can include those who may also have a cancer. The subject treated by these methods directed to delaying the onset or progression and/or treatment of age-related diseases may be in partial or complete remission (also called cancer remission). In other certain embodiments, the subject to be treated with DGLA, and/or GLA, and/or a D5D inhibitor does not have a cancer (i.e., the subject has not been diagnosed as having a cancer by a person skilled in the medical art).

### Cardiovascular Diseases and Disorders.

In another embodiment, the senescence-associated disease or disorder treated by the methods described herein is a cardiovascular disease. The cardiovascular disease may be any one or more of angina, arrhythmia, atherosclerosis, cardiomyopathy, congestive heart failure, coronary artery disease (CAD), carotid artery disease, endocarditis, heart attack (coronary thrombosis, myocardial infarction [MI]), high blood pressure/hypertension, aortic aneurysm, brain aneurysm, cardiac fibrosis, cardiac diastolic dysfunction, hypercholesterolemia/hyperlipidemia, mitral valve prolapse, peripheral vascular disease (e.g., peripheral artery disease (PAD)), cardiac stress resistance, and stroke.

In certain embodiments, methods are provided for treating senescence-associated cardiovascular disease that is associated with or caused by arteriosclerosis (i.e., hardening of the arteries). The cardiovascular disease may be any one or more of atherosclerosis (e.g., coronary artery disease (CAD) and carotid artery disease); angina, congestive heart failure, and peripheral vascular disease (e.g., peripheral artery disease (PAD)). The methods for treating a cardiovascular disease that is associated with or caused by arteriosclerosis may reduce the likelihood of occurrence of high blood pressure/hypertension, angina, stroke, and heart attack (i.e., coronary thrombosis, myocardial infarction (MI)). In certain embodiments, methods are provided for stabilizing atherosclerotic plaque(s) in a blood vessel (e.g., artery) of a subject, thereby reducing the likelihood of occurrence or delaying the occurrence of a thrombotic event, such as stroke or MI. In certain embodiments, these methods comprising administration of DGLA, and/or GLA, and/or a D5D inhibitor alone or in combination with one or more additional senolytic agents to reduce (i.e., cause decrease of) the lipid content of an atherosclerotic plaque in a blood vessel (e.g., artery) of the subject and/or increase the fibrous cap thickness (i.e., cause an increase, enhance or promote thickening and stabilization of the fibrous cap).

Atherosclerosis is characterized by patchy intimal plaques (atheromas) that encroach on the lumen of medium-sized and large arteries; the plaques contain lipids, inflammatory cells, smooth muscle cells, and connective tissue. Atherosclerosis can affect large and medium-sized arteries, including the coronary, carotid, and cerebral arteries, the aorta and its branches, and major arteries of the extremities. Atherosclerosis is characterized by patchy intimal plaques (atheromas) that encroach on the lumen of medium-sized and large arteries; the plaques contain lipids, inflammatory cells, smooth muscle cells, and connective tissue.

In one embodiment, methods are provided for inhibiting the formation of atherosclerotic plaques (or reducing, diminishing, causing decrease in formation of atherosclerotic plaques) by administering DGLA, and/or GLA, and/or a D5D inhibitor alone or in combination with one or more additional senolytic agents. In other embodiments, methods are provided for reducing (decreasing, diminishing) the amount (i.e., level) of plaque. Reduction in the amount of plaque in a blood vessel (e.g., artery) may be determined, for example, by a decrease in surface area of the plaque, or by a decrease in the extent or degree (e.g., percent) of occlusion of a blood vessel (e.g., artery), which can be determined by angiography or other visualizing methods used in the cardiovascular art. Also provided herein are methods for increasing the stability (or improving, promoting, or enhancing stability (see below) of atherosclerotic plaques that are present in one or more blood vessels (e.g., one or more arteries) of a subject, which methods comprise administering to the subject DGLA, and/or GLA, and/or a D5D inhibitor alone or in combination with one or more additional senolytic agents.

Atherosclerosis is often referred to as a “hardening” or furring of the arteries and is caused by the formation of multiple atheromatous plaques within the arteries. Atherosclerosis (also called arteriosclerotic vascular disease or ASVD herein and in the art) is a form of arteriosclerosis in which an artery wall thickens. Symptoms develop when growth or rupture of the plaque reduces or obstructs blood flow; and the symptoms may vary depending on which artery is affected. Atherosclerotic plaques may be stable or unstable. Stable plaques regress, remain static, or grow slowly, sometimes over several decades, until they may cause stenosis or occlusion. Unstable plaques are vulnerable to spontaneous erosion, fissure, or rupture, causing acute thrombosis, occlusion, and infarction long before they cause hemodynamically significant stenosis. Most clinical events result from unstable plaques, which do not appear severe on angiography; thus, plaque stabilization may be a way to reduce morbidity and mortality. Plaque rupture or erosion can lead to major cardiovascular events such as acute coronary syndrome and stroke (see, e.g., Du et al. (2014) BMC Cardiovascular Disorders 14: 83; Grimm et al. (2102) J. Cardiovasc. Magn. Res. 14: 80). Disrupted plaques were found to have a greater content of lipid, macrophages, and had a thinner fibrous cap than intact plaques (see, e.g., Felton et al.), 1007 Arteriosclerosis, Thrombosis, and Vascular Biology 17: 1337-1345).

Atherosclerosis is a syndrome affecting arterial blood vessels due in significant part to a chronic inflammatory response of white blood cells in the walls of arteries. This is promoted by low-density lipoproteins (LDL, plasma proteins that carry cholesterol and triglycerides) in the absence of adequate removal of fats and cholesterol from macrophages by functional high-density lipoproteins (HDL). The earliest visible lesion of atherosclerosis is the “fatty streak,” which is an accumulation of lipid-laden foam cells in the intimal layer of the artery. The hallmark of atherosclerosis is atherosclerotic plaque, which is an evolution of the fatty streak and has three major components: lipids (e.g., cholesterol and triglycerides); inflammatory cells and smooth muscle cells; and a connective tissue matrix that may contain thrombi in various stages of organization and calcium deposits. Within the outer-most and oldest plaque, calcium and other crystallized components (e.g., microcalcification) from dead cells can be found. Microcalcification and properties related thereto are also thought to contribute to plaque instability by increasing plaque stress (see, e.g., Bluestein et al., (2008) J. Biomech. 41(5): 1111-1118; Cilla et al. (2013) J. Engineering in Med. 227: 588-599). Fatty streaks reduce the elasticity of the artery walls, but may not affect blood flow for years because the artery muscular wall accommodates by enlarging at the locations of plaque. Lipid-rich atheromas are at increased risk for plaque rupture and thrombosis (see, e.g., Felton et al., supra; Fuster et al. (2005) J. Am. Coll. Cardiol. 46: 1209-1218). Reports have found that of all plaque components, the lipid core exhibits the highest thrombogenic activity (see, e.g., Fernandez-Ortiz et al. (1994) J. Am. Coll. Cardiol. 23: 1562-1569). Within major arteries in advanced disease, the wall stiffening may also eventually increase pulse pressure.

A vulnerable plaque that may lead to a thrombotic event (stroke or MI) and is sometimes described as a large, soft lipid pool covered by a thin fibrous cap (see, e.g., Li et al. (2006) Stroke, 37: 1195-1199; Trivedi et al. (2004) Neuroradiology, 46: 738-743). An advanced characteristic feature of advance atherosclerotic plaque is irregular thickening of the arterial intima by inflammatory cells, extracellular lipid (atheroma) and fibrous tissue (sclerosis) (see, e.g., Newby et al. (1999) Cardiovasc. Res. 41: 345-360). Fibrous cap formation is believed to occur from the migration and proliferation of vascular smooth muscle cells and from matrix deposition (see, e.g., Ross (1993) Nature, 362: 801-809; Sullivan et al. (2013) J. Angiology at dx.doi.org/10.1155/2013/592815). A thin fibrous cap contributes to instability of the plaque and to increased risk for rupture (see, e.g., Li et al., supra).

Both proinflammatory macrophages (M1) and anti-inflammatory macrophages (M2) can be found in arteriosclerotic plaque. The contribution of both types of cells to plaque instability is a subject of active investigation, with results suggesting that an increased level of the MI type versus the M2 type correlates with increased instability of plaque (see, e.g., Medbury et al. (2013) Int. Angiol. 32: 74-84; Lee et al. (2013) Am. J. Clin. Pathol. 139: 317-322; Martinet et al. (2007) Cir. Res. 751-753).

Subjects suffering from cardiovascular disease can be identified using standard diagnostic methods known in the art for cardiovascular disease. Generally, diagnosis of atherosclerosis and other cardiovascular disease is based on symptoms (e.g., chest pain or pressure (angina), numbness or weakness in arms or legs, difficulty speaking or slurred speech, drooping muscles in face, leg pain, high blood pressure, kidney failure and/or erectile dysfunction), medical history, and/or physical examination of a patient. Diagnosis may be confirmed by angiography, ultrasonography, or other imaging tests. Subjects at risk of developing cardiovascular disease include those having any one or more of predisposing factors, such as a family history of cardiovascular disease and those having other risk factors (i.e., predisposing factors) such as high blood pressure, dyslipidemia, high cholesterol, diabetes, obesity and cigarette smoking, sedentary lifestyle, and hypertension. In a certain embodiment, the cardiovascular disease that is a senescence cell associated disease/disorder is atherosclerosis.

The effectiveness of DGLA, and/or GLA, and/or a D5D inhibitor alone or in combination with one or more additional senolytic agents for treating or preventing (i.e., reducing or decreasing the likelihood of developing or occurrence of) a cardiovascular disease (e.g., atherosclerosis) can readily be determined by a person skilled in the medical and clinical arts. One or any combination of diagnostic methods, including physical examination, assessment and monitoring of clinical symptoms, and performance of analytical tests and methods described herein and practiced in the art (e.g., angiography, electrocardiogramon-stress test), may be used for monitoring the health status of the subject. The effects of DGLA, and/or GLA, and/or a D5D inhibitor alone or in combination with one or more additional senolytic agents or pharmaceutical compositions comprising same can be analyzed using techniques known in the art, such as comparing symptoms of patients suffering from or at risk of cardiovascular disease that have received the treatment with those of patients without such a treatment or with placebo treatment.

### Inflammatory and Autoimmune Diseases and Disorders.

In certain embodiments, a senescence-associated disease or disorder is an inflammatory disease or disorder, such as by way of a non-limiting example, osteoarthritis, that may be treated or prevented (i.e., likelihood of occurrence is reduced) according to the methods described herein that comprise administration of DGLA, and/or GLA, and/or a D5D inhibitor alone or in combination with one or more additional senolytic agents. Other inflammatory or autoimmune diseases or disorders that may be treated by administering DGLA, and/or GLA, and/or a D5D inhibitor alone or in combination with one or more additional senolytic agents described herein include osteoporosis, psoriasis, oral mucositis, rheumatoid arthritis, inflammatory bowel disease, eczema, kyphosis, herniated intervertebral disc, and the pulmonary diseases COPD and idiopathic pulmonary fibrosis.

Osteoarthritis is a degenerative joint disease characterized by fibrillation of the cartilage at sites of high mechanical stress, bone sclerosis, and thickening of the synovium and the joint capsule. Fibrillation is a local surface disorganization involving splitting of the superficial layers of the cartilage. The early splitting is tangential with the cartilage surface, following the axes of the predominant collagen bundles. Collagen within the cartilage becomes disorganized, and proteoglycans are lost from the cartilage surface. In the absence of protective and lubricating effects of proteoglycans in a joint, collagen fibers become susceptible to degradation, and mechanical destruction ensues. Predisposing risk factors for developing osteoarthritis include increasing age, obesity, previous joint injury, overuse of the joint, weak thigh muscles, and genetics. It is a common cause of chronic disability in the elderly. Symptoms of osteoarthritis include sore or stiff joints, particularly the hips, knees, and lower back, after inactivity or overuse; stiffness after resting that goes away after movement; and pain that is worse after activity or toward the end of the day. Osteoarthritis may also affect the neck, small finger joints, the base of the thumb, ankle, and big toe.

Chronic inflammation is thought to be the main age-related factor that contributes to osteoarthritis. In combination with aging, joint overuse and obesity appear to promote osteoarthritis.

Unexpectedly, by selectively killing senescent cells it is believed that DGLA, and/or GLA, and/or a D5D inhibitor alone, or in combination with one or more senolytic agents can prevent (e.g., reduces the likelihood of occurrence), reduces or inhibits loss or erosion of proteoglycan layers in a joint, reduces inflammation in the affected joint, and promotes (i.e., stimulates, enhances, induces) production of collagen (e.g., type 2 collagen). Removal of senescent cells causes a reduction in the amount (i.e., level) of inflammatory cytokines, such as IL-6, produced in a joint and reduction of inflammation. Methods are provided herein for treating osteoarthritis, for selectively killing senescent cells in an osteoarthritic joint of a subject, and/or inducing collagen (such as Type 2 collagen) production in the joint of a subject in need thereof by administering DGLA, and/or GLA, and/or a D5D inhibitor alone or in combination with one or more additional senolytic agents (which may be combined with at least one pharmaceutically acceptable excipient to form a pharmaceutical composition) to the subject. DGLA, and/or GLA, and/or a D5D inhibitor alone or in combination with one or more additional senolytic agents also may be used for decreasing (inhibiting, reducing) production of metalloproteinase 13 (MMP-13), which degrades collagen in a joint, and for restoring proteoglycan layer or inhibiting loss and/or degradation of the proteoglycan layer. Treatment with DGLA alone or in combination with one or more additional senolytic agents thereby also prevents (i.e., reduces likelihood of occurrence of), inhibits, or decreases erosion, or slows (i.e., decreases rate) erosion of the bone. As described in detail herein, in certain embodiments, DGLA, and/or GLA, and/or a D5D inhibitor alone or in combination with one or more additional senolytic agents is administered directly to an osteoarthritic joint (e.g., by intra-articular, topical, transdermal, intradermal, or subcutaneous delivery). Treatment with DGLA, and/or GLA, and/or a D5D inhibitor alone or in combination with one or more additional senolytic agents may also restore, improve, or inhibit deterioration of strength of a joint. In addition, the methods comprising administering DGLA, and/or GLA, and/or a D5D inhibitor alone or in combination with one or more additional senolytic agents can reduce joint pain and are therefore useful for pain management of osteoarthritic joints.

The effectiveness of DGLA, and/or GLA, and/or a D5D inhibitor alone or in combination with one or more additional senolytic agents for treatment or prophylaxis of osteoarthritis in a subject and monitoring of a subject who receives DGLA, and/or GLA, and/or a D5D inhibitor alone or in combination with one or more additional senolytic agents can readily be determined by a person skilled in the medical and clinical arts. One or any combination of diagnostic methods, including physical examination (such as determining tenderness, swelling or redness of the affected joint), assessment and monitoring of clinical symptoms (such as pain, stiffness, mobility), and performance of analytical tests and methods described herein and practiced in the art (e.g., determining the level of inflammatory cytokines or chemokines; X-ray images to determine loss of cartilage as shown by a narrowing of space between the bones in a joint; magnetic resonance imaging (MRI), providing detailed images of bone and soft tissues, including cartilage), may be used for monitoring the health status of the subject. The effects of the treatment of DGLA, and/or GLA, and/or a D5D inhibitor alone or in combination with one or more additional senolytic agents can be analyzed by comparing symptoms of patients suffering from or at risk of an inflammatory disease or disorder, such as osteoarthritis, who have received the treatment with those of patients who have not received such a treatment or who have received a placebo treatment.

In certain embodiments, DGLA, and/or GLA, and/or a D5D inhibitor alone or in combination with one or more additional senolytic agents may be used for treating and/or preventing (i.e., decreasing or reducing the likelihood of occurrence) rheumatoid arthritis (RA). Dysregulation of innate and adaptive immune responses characterize rheumatoid arthritis (RA), which is an autoimmune disease the incidence of which increases with age. Rheumatoid arthritis is a chronic inflammatory disorder that typically affects the small joints in hands and feet. Whereas osteoarthritis results from, at least in part, wear and tear of a joint, rheumatoid arthritis affects the lining of joints, resulting in a painful swelling that can lead to bone erosion and joint deformity. RA can sometimes also affect other organs of the body, such as the skin, eyes, lungs and blood vessels. RA can occur in a subject at any age; however, RA usually begins to develop after age 40. The disorder is much more common in women. In certain embodiments of the methods described herein, RA is excluded.

Chronic inflammation may also contribute to other age-related or aging related diseases and disorders, such as kyphosis and osteoporosis. Kyphosis is a severe curvature in the spinal column, and it is frequently seen with normal and premature aging (see, e.g., Katzman et al. (2010) J. Orthop. Sports Phys. Ther. 40: 352-360). Age-related kyphosis often occurs after osteoporosis weakens spinal bones to the point that they crack and compress. A few types of kyphosis target infants or teens. Severe kyphosis can affect lungs, nerves, and other tissues and organs, causing pain and other problems. Kyphosis has been associated with cellular senescence. Characterizing the capability of a DGLA, and/or GLA, and/or a D5D inhibitor alone or in combination with one or more additional senolytic agents for treating kyphosis may be determined in pre-clinical animal models used in the art. By way of example, TTD mice develop kyphosis (see, e.g., de Boer et al. (2002) Science, 296: 1276-1279); other mice that may be used include BubR1^{H/H} mice, which are also known to develop kyphosis (see, e.g., Baker et al. (2011) Nature, 479: 232-36). Kyphosis formation is visually measured over time. The level of senescent cells decreased by treatment with DGLA, and/or GLA, and/or a D5D inhibitor alone or in combination with one or more additional senolytic agents can be determined by detecting the presence of one or more senescent cell associated markers such as by SA-β-Gal staining.

Osteoporosis is a progressive bone disease that is characterized by a decrease in bone mass and density that may lead to an increased risk of fracture. Bone mineral density (BMD) is reduced, bone microarchitecture deteriorates, and the amount and variety of proteins in bone are altered. Osteoporosis is typically diagnosed and monitored by a bone mineral density test. Post-menopausal women or women who have reduced estrogen are most at risk. While both men and women over 75 are at risk, women are twice as likely to develop osteoporosis than men. The level of senescent cells decreased by treatment with DGLA, and/or GLA, and/or a D5D inhibitor alone or in combination with one or more additional senolytic agents can be determined by detecting the presence of one or more senescent cell associated markers such as by SA-β-Gal staining.

In still other embodiments, an inflammatory/autoimmune disorder that may be treated or prevented (i.e., likelihood of occurrence is reduced) with DGLA, and/or GLA, and/or a D5D inhibitor alone or in combination with one or more additional senolytic agents includes irritable bowel syndrome (IBS) and inflammatory bowel diseases, such as ulcerative colitis and Crohn's disease. Inflammatory bowel disease (IBD) involves chronic inflammation of all or part of the digestive tract. In addition to life-threatening complications arising from IBD, the disease can be painful and debilitating. Ulcerative colitis is an inflammatory bowel disease that causes long-lasting inflammation in part of the digestive tract. Symptoms usually develop over time, rather than suddenly. Ulcerative colitis usually affects only the innermost lining of the large intestine (colon) and rectum. Crohn's disease is an inflammatory bowel disease that causes inflammation anywhere along the lining of your digestive tract, and often extends deep into affected tissues. This can lead to abdominal pain, severe diarrhea, and malnutrition. The inflammation caused by Crohn's disease can involve different areas of the digestive tract. Diagnosis and monitoring of the diseases is performed according to methods and diagnostic tests routinely practiced in the art, including blood tests, colonoscopy, flexible sigmoidoscopy, barium enema, CT scan, MRI, endoscopy, and small intestine imaging.

In other embodiments, the methods described herein may be useful for treating a subject who has herniated or degenerated intervertebral discs. Subjects with these discs exhibit elevated presence of cell senescence in the blood, in vessel walls (see e.g., Roberts et al. (2006) Eur. Spine J. 15 Suppl 3: S312-316) and/or in the discs (Patil et al. (2019) Aging Cell 18: e12927). Symptoms of a herniated or degenerate intervertebral disc may include pain, numbness or tingling, or weakness in an arm or leg. Increased levels of proinflammatory molecules and matrix metalloproteases are also found in aging and degenerating discs tissues, suggesting a role for senescence cells (see e.g., Chang-Qing et al. (2007) Ageing Res. Rev. 6: 247-61). Animal models may be used to characterize the effectiveness of DGLA, and/or GLA, and/or a D5D inhibitor alone or in combination with one or more additional senolytic agents in treating herniated or degenerated (Patil et al. (2019) Aging Cell, 18: e12927) intervertebral discs; degeneration of the intervertebral disc is induced in mice by compression and disc strength evaluated (see e.g., Lotz et al. (1998) Spine (Philadelphia Pa. 1976). 23: 2493-506).

Other inflammatory or autoimmune diseases that may be treated or prevented (e.g., likelihood of occurrence is reduced) by using DGLA, and/or GLA, and/or a D5D inhibitor alone or in combination with one or more additional senolytic agents include eczema, psoriasis, osteoporosis, and pulmonary diseases (e.g., chronic obstructive pulmonary disease (COPD), idiopathic pulmonary fibrosis (IPF), asthma), inflammatory bowel disease, and mucositis (including oral mucositis, which in some instances is induced by radiation). Certain fibrosis or fibrotic conditions of organs such as renal fibrosis, liver fibrosis, pancreatic fibrosis, cardiac fibrosis, skin wound healing, and oral submucous fibrosis may be treated with DGLA, and/or GLA, and/or a D5D inhibitor alone or in combination with one or more additional senolytic agents.

In certain embodiments, the senescent cell associated disorder is an inflammatory disorder of the skin, such as by way of a non-limiting examples, psoriasis and eczema that may be treated or prevented (i.e., likelihood of occurrence is reduced) according to the methods described herein that comprise administration of DGLA, and/or GLA, and/or a D5D inhibitor alone or in combination with one or more additional senolytic agents. Psoriasis is characterized by abnormally excessive and rapid growth of the epidermal layer of the skin. A diagnosis of psoriasis is usually based on the appearance of the skin. Skin characteristics typical for psoriasis are scaly red plaques, papules, or patches of skin that may be painful and itch. In psoriasis, cutaneous and systemic overexpression of various proinflammatory cytokines is observed such as IL-6, a key component of the SASP. Eczema is an inflammation of the skin that is characterized by redness, skin swelling, itching and dryness, crusting, flaking, blistering, cracking, oozing, or bleeding. The effectiveness of DGLA, and/or GLA, and/or a D5D inhibitor alone or in combination with one or more additional senolytic agents for treatment of psoriasis and eczema and monitoring of a subject who receives such the senolytic agent(s) can be readily determined by a person skilled in the medical or clinical arts. One or any combination of diagnostic methods, including physical examination (such as skin appearance), assessment of monitoring of clinical symptoms (such as itching, swelling, and pain), and performance of analytical tests and methods described herein and practiced in the art (i.e., determining the level of pro-inflammatory cytokines).

Other immune disorders or conditions that may be treated or prevented (i.e., likelihood of occurrence is reduced) with DGLA, and/or GLA, and/or a D5D inhibitor alone or in combination with one or more additional senolytic agents include conditions resulting from a host immune response to an organ transplant (e.g., kidney, bone marrow, liver, lung, or heart transplant), such as rejection of the transplanted organ. In certain embodiments DGLA, and/or GLA, and/or a D5D inhibitor alone or in combination with one or more additional senolytic agents may be used for treating or reducing the likelihood of occurrence of graft-vs-host disease.

### Pulmonary Diseases and Disorders.

In one embodiment, methods are provided for treating or preventing (i.e., reducing the likelihood of occurrence of) a senescence-associated disease or disorder that is a pulmonary disease or disorder by killing senescent cells (i.e., established senescent cells) associated with the disease or disorder in a subject who has the disease or disorder by administering DGLA, and/or GLA, and/or a D5D inhibitor alone or in combination with one or more additional senolytic agents. Senescence associated pulmonary diseases and disorders include, for example, idiopathic pulmonary fibrosis (IPF), chronic obstructive pulmonary disease (COPD), asthma, cystic fibrosis, bronchiectasis, and emphysema.

COPD is a lung disease defined by persistently poor airflow resulting from the breakdown of lung tissue (emphysema) and the dysfunction of the small airways (obstructive bronchiolitis). Primary symptoms of COPD include shortness of breath, wheezing, chest tightness, chronic cough, and excess sputum production. Elastase from cigarette smoke-activated neutrophils and macrophages disintegrates the extracellular matrix of alveolar structures, resulting in enlarged air spaces and loss of respiratory capacity (see, e.g., Shapiro et al., Am. J. Respir. Cell Mol. Biol. 32, 367-372 (2005)). COPD is most commonly caused by tobacco smoke (including cigarette smoke, cigar smoke, secondhand smoke, pipe smoke), occupational exposure (e.g., exposure to dust, smoke or fumes), and pollution, occurring over decades thereby implicating aging as a risk factor for developing COPD.

The processes involved in causing lung damage include, for example, oxidative stress produced by the high concentrations of free radicals in tobacco smoke; cytokine release due to inflammatory response to irritants in the airway; and impairment of anti-protease enzymes by tobacco smoke and free radicals, allowing proteases to damage the lungs. Genetic susceptibility can also contribute to the disease. In about 1% of people with COPD, the disease results from a genetic disorder that causes low level production of alpha-1-antitrypsin in the liver. The enzyme is normally secreted into the bloodstream to help protect the lungs.

Pulmonary fibrosis is a chronic and progressive lung disease characterized by stiffening and scarring of the lung, which may lead to respiratory failure, lung cancer, and heart failure. Fibrosis is associated with repair of epithelium. Fibroblasts are activated, production of extracellular matrix proteins is increased, and transdifferentiation to contractile myofibroblasts contribute to wound contraction. A provisional matrix plugs the injured epithelium and provides a scaffold for epithelial cell migration, involving an epithelial-mesenchymal transition (EMT). Blood loss associated with epithelial injury induces platelet activation, production of growth factors, and an acute inflammatory response. Normally, the epithelial barrier heals and the inflammatory response resolves. However, in fibrotic disease the fibroblast response continues, resulting in unresolved wound healing. Formation of fibroblastic foci is a feature of the disease, reflecting locations of ongoing fibrogenesis. As the name connotes, the etiology of IPF is unknown. The involvement of cellular senescence in IPF is suggested by the observations that the incidence of the disease increases with age and that lung tissue in IPF patients is enriched for SA-β-Gal-positive cells and contains elevated levels of the senescence marker p21 (see, e.g., Minagawa et al., (2011) Am. J. Physiol. Lung Cell. Mol. Physiol. 300: L391-L401; Naylor et al., supra). Short telomeres are a risk factor common to both IPF and cellular senescence (see, e.g., Alder et al. (2008) Proc. Natl. Acad. Sci. USA, 105:13051-13056). Without wishing to be bound by theory, the contribution of cellular senescence to IPF is suggested by the report that SASP components of senescent cells, such as IL-6, IL-8, and IL-1β, promotes fibroblast-to-myofibroblast differentiation and epithelial-mesenchymal transition, resulting in extensive remodeling of the extracellular matrix of the alveolar and interstitial spaces (see, e.g., Minagawa et al., supra; Wiley et al. (2019) J. Clin. Invest. Insight. 4: e130056).

Subjects at risk of developing pulmonary fibrosis include those exposed to environmental or occupational pollutants, such as asbestosis and silicosis; who smoke cigarettes; having some typical connective tissue diseases such as rheumatoid arthritis, systemic lupus erythematosus and scleroderma; having other diseases that involve connective tissue, such as sarcoidosis and Wegener's granulomatosis; having infections; taking certain medications (e.g., amiodarone, bleomycin, busufan, methotrexate, and nitrofurantoin); those subject to radiation therapy to the chest; and those whose family member has pulmonary fibrosis.

Symptoms of COPD may include any one of shortness of breath, especially during physical activities; wheezing; chest tightness; having to clear your throat first thing in the morning because of excess mucus in the lungs; a chronic cough that produces sputum that may be clear, white, yellow or greenish; blueness of the lips or fingernail beds (cyanosis); frequent respiratory infections; lack of energy; unintended weight loss (observed in later stages of disease). Subjects with COPD may also experience exacerbations, during which symptoms worsen and persist for days or longer. Symptoms of pulmonary fibrosis are known in the art and include shortness of breath, particularly during exercise; dry, hacking cough; fast, shallow breathing; gradual unintended weight loss; tiredness; aching joints and muscles; and clubbing (widening and rounding of the tips of the fingers or toes).

Subjects suffering from COPD or pulmonary fibrosis can be identified using standard diagnostic methods routinely practiced in the art. Monitoring the effect DGLA, and/or GLA, and/or a D5D inhibitor alone or in combination with one or more additional senolytic agents administered to a subject who has or who is at risk of developing a pulmonary disease may be performed using the methods typically used for diagnosis. Generally, one or more of the following exams or tests may be performed: physical exam, patient's medical history, patient's family's medical history, chest X-ray, lung function tests (such as spirometry), blood test (e.g., arterial blood gas analysis), bronchoalveolar lavage, lung biopsy, CT scan, and exercise testing.

Other pulmonary diseases or disorders that may be treated by using DGLA, and/or GLA, and/or a D5D inhibitor alone or in combination with one or more additional senolytic agents include, for example, emphysema, asthma, bronchiectasis, and cystic fibrosis (see, e.g., Fischer et al. (2013) Am. J. Physiol. Lung Cell Mol. Physiol. 304(6): L394-400). These diseases may also be exacerbated by tobacco smoke (including cigarette smoke, cigar smoke, secondhand smoke, pipe smoke), occupational exposure (e.g., exposure to dust, smoke, or fumes), infection, and/or pollutants that induce cells into senescence and thereby contribute to inflammation. Emphysema is sometimes considered as a subgroup of COPD.

Bronchiectasis results from damage to the airways that causes them to widen and become flabby and scarred. Bronchiectasis usually is caused by a medical condition that injures the airway walls or inhibits the airways from clearing mucus. Examples of such conditions include cystic fibrosis and primary ciliary dyskinesia (PCD). When only one part of the lung is affected, the disorder may be caused by a blockage rather than a medical condition.

The methods described herein for treating or preventing (i.e., reducing the likelihood of occurrence of) a senescence associated pulmonary disease or disorder may also be used for treating a subject who is aging and has loss (or degeneration) of pulmonary function (i.e., declining or impaired pulmonary function compared with a younger subject) and/or degeneration of pulmonary tissue. The respiratory system undergoes various anatomical, physiological and immunological changes with age. The structural changes include chest wall and thoracic spine deformities that can impair the total respiratory system compliance resulting in increased effort to breathe. The respiratory system undergoes structural, physiological, and immunological changes with age. An increased proportion of neutrophils and lower percentage of macrophages can be found in bronchoalveolar lavage (BAL) of older adults compared with younger adults. Persistent low-grade inflammation in the lower respiratory tract can cause proteolytic and oxidant-mediated injury to the lung matrix resulting in loss of alveolar unit and impaired gas exchange across the alveolar membrane seen with aging. Sustained inflammation of the lower respiratory tract may predispose older adults to increased susceptibility to toxic environmental exposure and accelerated lung function decline. (See, for example, Sharma et al., Clinical Interventions in Aging 1:253-60 (2006)). Oxidative stress exacerbates inflammation during aging (see, e.g., Brod (2000) Inflamm. Res. 49: 561-570; Hendel et al. (2010) Cell Death and Differentiation, 17: 596-606). Alterations in redox balance and increased oxidative stress during aging precipitate the expression of cytokines, chemokines, and adhesion molecules, and enzymes (see, e.g., Chung et al. (2009) Ageing Res. Rev. 8: 18-30). Constitutive activation and recruitment of macrophages, T cells, and mast cells foster release of proteases leading to extracellular matrix degradation, cell death, remodeling, and other events that can cause tissue and organ damage during chronic inflammation (see, e.g., Demedts et al. (2006) Respir. Res. 7: 53-63). By administering DGLA, and/or GLA, and/or a D5D inhibitor alone or in combination with one or more additional senolytic agents to an aging subject (which includes a middle-aged adult who is asymptomatic), the decline in pulmonary function may be decelerated or inhibited by killing and removing senescent cells from the respiratory tract.

The effectiveness of DGLA, and/or GLA, and/or a D5D inhibitor alone or in combination with one or more additional senolytic agents can readily be determined by a person skilled in the medical and clinical arts. One or any combination of diagnostic methods, including physical examination, assessment and monitoring of clinical symptoms, and performance of analytical tests and methods described herein, may be used for monitoring the health status of the subject. The effects of the treatment with DGLA, and/or GLA, and/or a D5D inhibitor alone or in combination with one or more additional senolytic agents or pharmaceutical composition comprising the agent can be analyzed using techniques known in the art, such as comparing symptoms of patients suffering from or at risk of the pulmonary disease that have received the treatment with those of patients without such a treatment or with placebo treatment. In addition, methods and techniques that evaluate mechanical functioning of the lung, for example, techniques that measure lung capacitance, elastance, and airway hypersensitivity may be performed. To determine lung function and to monitor lung function throughout treatment, any one of numerous measurements may be obtained, expiratory reserve volume (ERV), forced vital capacity (FVC), forced expiratory volume (FEV) (e.g., FEV in one second, FEV1), FEV1/FEV ratio, forced expiratory flow 25% to 75%, and maximum voluntary ventilation (MVV), peak expiratory flow (PEF), slow vital capacity (SVC). Total lung volumes include total lung capacity (TLC), vital capacity (VC), residual volume (RV), and functional residual capacity (FRC). Gas exchange across alveolar capillary membrane can be measured using diffusion capacity for carbon monoxide (DLCO). Peripheral capillary oxygen saturation (SpO₂) can also be measured; normal oxygen levels are typically between 95% and 100%. An SpO₂ level below 90% suggests the subject has hypoxemia. Values below 80% are considered critical and requiring intervention to maintain brain and cardiac function and avoid cardiac or respiratory arrest.

### Neurological Diseases and Disorders.

Senescence-associated diseases or disorders treatable by administering DGLA, and/or GLA, and/or a D5D inhibitor alone or in combination with one or more additional senolytic agents include neurological diseases or disorders. Such senescence-associated diseases and disorders include Parkinson's disease, Alzheimer's disease (and other dementias), motor neuron dysfunction (MND), mild cognitive impairment (MCI), Huntington's disease, and diseases and disorders of the eyes, such as age-related macular degeneration. Other diseases of the eye that are associated with increasing age are glaucoma, vision loss, presbyopia, and cataracts.

Parkinson's disease (PD) is the second most common neurodegenerative disease. It is a disabling condition of the brain characterized by slowness of movement (bradykinesia), shaking, stiffness, and in the later stages, loss of balance. Many of these symptoms are due to the loss of certain nerves in the brain, which results in a lack of dopamine. This disease is characterized by neurodegeneration, such as the loss of about 50% to 70% of the dopaminergic neurons in the substantia nigra pars compacta, a profound loss of dopamine in the striatum, and/or the presence of intracytoplasmic inclusions (Lewy bodies), which are composed mainly of alpha-synuclein and ubiquitin. Parkinson's disease also features locomotor deficits, such as tremor, rigidity, bradykinesia, and/or postural instability. Subjects at risk of developing Parkinson's disease include those having a family history of Parkinson's disease and those exposed to pesticides (e.g., rotenone or paraquat), herbicides (e.g., agent orange), or heavy metals. Senescence of dopamine-producing neurons is thought to contribute to the observed cell death in PD through the production of reactive oxygen species (see, e.g., Cohen et al. (1983) J. Neural Transm. Suppl. 19: 89-103); therefore, the methods described herein are useful for treatment and prophylaxis of Parkinson's disease.

Methods for detecting, monitoring or quantifying neurodegenerative deficiencies and/or locomotor deficits associated with Parkinson's disease are known in the art, such as histological studies, biochemical studies, and behavioral assessment (see, e.g., U.S. Application Publication No. 2012/0005765). Symptoms of Parkinson's disease are known in the art and include, but are not limited to, difficulty starting or finishing voluntary movements, jerky, stiff movements, muscle atrophy, shaking (tremors), and changes in heart rate, but normal reflexes, bradykinesia, and postural instability. There is a growing recognition that people diagnosed with Parkinson's disease may have cognitive impairment, including mild cognitive impairment, in addition to their physical symptoms.

Alzheimer's disease (AD) is a neurodegenerative disease that shows a slowly progressive mental deterioration with failure of memory, disorientation, and confusion, leading to profound dementia. Age is the single greatest predisposing risk factor for developing AD, which is the leading cause of dementia in the elderly (see, e.g., Hebert, et al. (2003) Arch. Neural. 60: 1119-1122). Early clinical symptoms show remarkable similarity to mild cognitive impairment (see below). As the disease progresses, impaired judgment, confusion, behavioral changes, disorientation, and difficulty in walking and swallowing occur.

Alzheimer's disease is characterized by the presence of neurofibrillary tangles and amyloid (senile) plaques in histological specimens. The disease predominantly involves the limbic and cortical regions of the brain. The argyrophilic plaques containing the amyloidogenic Aβ fragment of amyloid precursor protein (APP) are scattered throughout the cerebral cortex and hippocampus. Neurofibrillary tangles are found in pyramidal neurons predominantly located in the neocortex, hippocampus, and nucleus basalis of Meynert. Other changes, such as granulovacuolar degeneration in the pyramidal cells of the hippocampus, and neuron loss and gliosis in the cortex and hippocampus, are observed. Subjects at risk of developing Alzheimer's disease include those of advanced age, those with a family history of Alzheimer's disease, those with genetic risk genes (e.g., ApoE4) or deterministic gene mutations (e.g., APP, PS1, or PS2), and those with history of head trauma or heart/vascular conditions (e.g., high blood pressure, heart disease, stroke, diabetes, high cholesterol).

A number of behavioral and histopathological assays are known in the art for evaluating Alzheimer's disease phenotype, for characterizing therapeutic agents, and assessing treatment. Histological analyses are typically performed postmortem. Histological analysis of Aβ levels may be performed using Thioflavin-S. Congo red, or anti-Aβ staining (e.g., 4G8, 10D5, or 6E10 antibodies) to visualize Aβ deposition on sectioned brain tissues (see, e.g., Holcomb et al. (1998) Nat. Med. 4: 97-100; Borchelt et al. (1997) Neuron, 19: 939-945; Dickson et al. (1988) Am. J. Path. 132: 86-101). In vivo methods of visualizing Aβ deposition in transgenic mice have been also described. BSB ((trans, trans)-1-bromo-2,5-bis-(3-hydroxycarbonyl-4-hydroxy)styrylbenzene) and PET tracer ¹¹C-labelled Pittsburgh Compound-B (PIB) bind to AP plaques (see, e.g., Skovronsky et al. (2000) Proc. Natl. Acad. Sci. USA, 97: 7609-7614; Klunk et al. (2004) Ann. Neurol. 55: 306-319). ¹⁹F-containing amyloidophilic Congo red-type compound FSB ((E,E)-1-fluoro-2,5-bis-(3-hydroxycarbonyl-4-hydroxy)styrylbenzene) allows visualization of Aβ plaques by MRI (see, e.g., Higuchi et al. (2005) Nat. Neurosci. 8: 527-533). Radiolabeled, putrescine-modified amyloid-beta peptide labels amyloid deposits in vivo in a mouse model of Alzheimer's disease (see, e.g., Wengenack et al. (2000) Nat. Biotechnol. 18: 868-872).

Increased glial fibrillary acidic protein (GFAP) in astrocytes is a marker for astroglial activation and gliosis during neurodegeneration. AP plaques are associated with GFAP-positive activated astrocytes, and may be visualized via GFAP staining (see, e.g., Nagele et al. (2004) Neurobiol. Aging, 25: 663-674; Mandybur et al. (1990) Neurology, 40: 635-639; Liang et al. (2010) J. Biol. Chem. 285: 27737-27744). Neurofibrillary tangles may be identified by immunohistochemistry using thioflavin-S fluorescent microscopy and Gallyas silver stains (see, e.g., Gotz et al. (2001) J. Biol. Chem. 276: 529-534; U.S. Pat. No. 6,664,443). Axon staining with electron microscopy and axonal transport studies may be used to assess neuronal degeneration (see, e.g., Ishihara et al. (1999) Neuron, 24: 751-762).

Subjects suffering from Alzheimer's disease can be identified using standard diagnostic methods known in the art for Alzheimer's disease. Generally, diagnosis of Alzheimer's disease is based on symptoms (e.g., progressive decline in memory function, gradual retreat from and frustration with normal activities, apathy, agitation or irritability, aggression, anxiety, sleep disturbance, dysphoria, aberrant motor behavior, disinhibition, social withdrawal, decreased appetite, hallucinations, dementia), medical history, neuropsychological tests, neurological and/or physical examination of a patient. Cerebrospinal fluid may also be for tested for various proteins that have been associated with Alzheimer pathology, including tau, amyloid beta peptide, and AD7C-NTP. Genetic testing is also available for early-onset familial Alzheimer disease (eFAD), an autosomal-dominant genetic disease. Clinical genetic testing is available for individuals with AD symptoms or at-risk family members of patients with early-onset disease. In the U.S., mutations for PS2, and APP may be tested in a clinical or federally approved laboratory under the Clinical Laboratory Improvement Amendments. A commercial test for PSI mutations is also available (Elan Pharmaceuticals).

The effectiveness of DGLA, and/or GLA, and/or a D5D inhibitor alone or in combination with one or more additional senolytic agents and monitoring of a subject who receives one or more senolytic agent(s) can readily be determined by a person skilled in the medical and clinical arts. One or any combination of diagnostic methods, including physical examination, assessment and monitoring of clinical symptoms, and performance of analytical tests and methods described herein, may be used for monitoring the health status of the subject. The effects of administering DGLA, and/or GLA, and/or a D5D inhibitor alone or in combination with one or more additional senolytic agents can be analyzed using techniques known in the art, such as comparing symptoms of patients suffering from or at risk of Alzheimer's disease that have received the treatment with those of patients without such a treatment or with placebo treatment.

### Mild Cognitive Impairment (MCI).

MCI is a brain-function syndrome involving the onset and evolution of cognitive impairments beyond those expected based on age and education of the individual, but which are not significant enough to interfere with this individual's daily activities. MCI is an aspect of cognitive aging that is considered to be a transitional state between normal aging and the dementia into which it may convert (see, Pepeu et al. (2004) Dialogues Clin. Neurosci. 6: 369-377). MCI that primarily affects memory is known as “amnestic MCI.” A person with amnestic MCI may start to forget important information that he or she would previously have recalled easily, such as recent events. Amnestic MCI is frequently seen as prodromal stage of Alzheimer's disease. MCI that affects thinking skills other than memory is known as “non-amnestic MCI.” This type of MCI affects thinking skills such as the ability to make sound decisions, judge the time or sequence of steps needed to complete a complex task, or visual perception. Individuals with non-amnestic MCI are believed to be more likely to convert to other types of dementias (e.g., dementia with Lewy bodies).

Persons in the medical art have a growing recognition that people diagnosed with Parkinson's disease may have MCI in addition to their physical symptoms. Recent studies show 20-30% of people with Parkinson's disease have MCI, and that their MCI tends to be non-amnestic. Parkinson's disease patients with MCI sometimes go on to develop full blown dementia (Parkinson's disease with dementia).

Methods for detecting, monitoring, quantifying or assessing neuropathological deficiencies associated with MCI are known in the art, including astrocyte morphological analyses, release of acetylcholine, silver staining for assessing histological hallmarks of neurodegeneration, and PiB PET imaging to detect beta amyloid deposits (see, e.g., U.S. Patent Application Publication No. 2012/0071468; Pepeu, 2004, supra). Methods for detecting, monitoring, quantifying or assessing behavioral deficiencies associated with MCI are also known in the art, including eight-arm radial maze paradigm, non-matching-to-sample task, allocentric place determination task in a water maze, Morris maze test, visuospatial tasks, and delayed response spatial memory task, olfactory novelty test (Id.).

### Motor Neuron Dysfunction (MND).

MND is a group of progressive neurological disorders that destroy motor neurons, the cells that control essential voluntary muscle activity such as speaking, walking, breathing and swallowing. It is classified according to whether degeneration affects upper motor neurons, lower motor neurons, or both. Examples of MNDs include, but are not limited to Amyotrophic Lateral Sclerosis (ALS), also known as Lou Gehrig's Disease, progressive bulbar palsy, pseudobulbar palsy, primary lateral sclerosis, progressive muscular atrophy, lower motor neuron disease, and spinal muscular atrophy (SMA) (e.g., SMA1 also called Werdnig-Hoffmann Disease, SMA2, SMA3 also called Kugelberg-Welander Disease, and Kennedy's disease), post-polio syndrome, and hereditary spastic paraplegia. In adults, the most common MND is amyotrophic lateral sclerosis (ALS), which affects both upper and lower motor neurons. It can affect the arms, legs, or facial muscles. Primary lateral sclerosis is a disease of the upper motor neurons, while progressive muscular atrophy affects only lower motor neurons in the spinal cord. In progressive bulbar palsy, the lowest motor neurons of the brain stem are most affected, causing slurred speech and difficulty chewing and swallowing. There are almost always mildly abnormal signs in the arms and legs. Patients with MND exhibit a phenotype of Parkinson's disease (e.g., having tremor, rigidity, bradykinesia, and/or postural instability). Methods for detecting, monitoring or quantifying locomotor and/or other deficits associated with Parkinson's diseases, such as MND, are known in the art (see, e.g., U.S. Application Publication No. 2012/0005765).

Methods for detecting, monitoring, quantifying or assessing motor deficits and histopathological deficiencies associated with MND are known in the art, including histopathological, biochemical, and electrophysiological studies and motor activity analysis (see, e.g., Rich et al. (2002) J. Neurophysiol. 88: 3293-3304; Appel et al., (1991) Proc. Natl. Acad. Sci. USA, 88: 647-51). Histopathologically, MNDs are characterized by death of motor neurons, progressive accumulation of detergent-resistant aggregates containing SOD1 and ubiquitin and aberrant neurofilament accumulations in degenerating motor neurons. In addition, reactive astroglia and microglia are often detected in diseased tissue. Patients with an MND show one or more motor deficits, including muscle weakness and wasting, uncontrollable twitching, spasticity, slow and effortful movements, and overactive tendon reflexes.

### Ophthalmic Diseases and Disorders

In certain embodiments, a senescence-associated disease or disorder is an ocular disease, disorder, or condition, for example, presbyopia, macular degeneration, or cataracts. In other certain embodiments, the senescence-associated disease or disorder is glaucoma. Macular degeneration is a neurodegenerative disease that causes the loss of photoreceptor cells in the central part of retina, called the macula. Macular degeneration generally is classified into two types: dry type and wet type. The dry form is more common than the wet, with about 90% of age-related macular degeneration (ARMD or AMD) patients diagnosed with the dry form. The wet form of the disease usually leads to more serious vision loss. While the exact causes of age-related macular degeneration are still unknown, the number of senescent retinal pigmented epithelial (RPE) cells increases with age. Age and certain genetic factors and environmental factors are risk factors for developing ARMD (see, e.g., Lyengar et al. (2004) Am. J. Hum. Genet. 74: 20-39); Kenealy et al. (2004) Mol. Vis. 10: 57-61; Gorin et al. (1999) Mol. Vis. 5: 29). Environment predisposing factors include omega-3 fatty acids intake (see, e.g., Christen et al. (2011) Arch Ophthalmol. 129: 921-929); estrogen exposure (see, e.g., Feshanich et al. (2008) Arch Ophthalmol. 126(4): 519-524); and increased serum levels of vitamin D (see, e.g., Millen, et al. (2011) Arch Ophthalmol. 129(4): 481-489). Genetic predisposing risk factors include reduced levels of Dicer1 (enzyme involved in maturation of micro RNA) in eyes of patients with dry AMD, and decreased micro RNAs contributes to a senescent cell profile; and DICER1 ablation induces premature senescence (see, e.g., Mudhasani et al. (2008) J. Cell Biol. 181(7): 1055-1063).

Dry ARMD is associated with atrophy of RPE layer, which causes loss of photoreceptor cells. The dry form of ARMD may result from aging and thinning of macular tissues and from deposition of pigment in the macula. Senescence appears to inhibit both replication and migration of RPE, resulting in permanent RPE depletion in the macula of dry AMD patients (see, e.g., Iriyama et al. (2008) J. Biol. Chem. 283: 11947-11953). With wet ARMD, new blood vessels grow beneath the retina and leak blood and fluid. This abnormal leaky choroidal neovascularization causes the retinal cells to die, creating blind spots in central vision. Different forms of macular degeneration may also occur in younger patients. Non-age related etiology may be linked to heredity, diabetes, nutritional deficits, head injury, infection, or other factors.

Declining vision noticed by the patient or by an ophthalmologist during a routine eye exam may be the first indicator of macular degeneration. The formation of exudates, or “drusen,” underneath the Bruch's membrane of the macula is often the first physical sign that macular degeneration may develop. Symptoms include perceived distortion of straight lines and, in some cases, the center of vision appears more distorted than the rest of a scene; a dark, blurry area or “white-out” appears in the center of vision; and/or color perception changes or diminishes. Diagnosing and monitoring of a subject with macular degeneration may be accomplished by a person skilled in the ophthalmic art according to art-accepted periodic eye examination procedures and report of symptoms by the subject.

Presbyopia is an age-related condition where the eye exhibits a progressively diminished ability to focus on near objects as the speed and amplitude of accommodation of a normal eye decreases with advancing age. Loss of elasticity of the crystalline lens and loss of contractility of the ciliary muscles have been postulated as its cause (see, e.g., Heys et al. (2004) Mol. Vis. 10: 956-963; Petrash (2013) Invest. Ophthalmol. Vis. Sci. 54: ORSF54-ORSF59). Age-related changes in the mechanical properties of the anterior lens capsule and posterior lens capsule suggest that the mechanical strength of the posterior lens capsule decreases significantly with age (see, e.g., Krag et al. (2003) Invest. Ophthalmol. Vis. Sci. 44: 691-696 (2003); Krag et al. (1997) Invest. Ophthalmol. Vis. Sci. 38: 357-463).

The laminated structure of the capsule also changes and may result, at least in part, from a change in the composition of the tissue (see, e.g., Krag et al., 1997, supra, and references cited therein). The major structural component of the lens capsule is basement membrane type IV collagen that is organized into a three-dimensional molecular network (see, e.g., Cummings et al. (2014) Connect. Tissue Res. 55: 8-12; Veis et al. (1981) Coll. Relat. Res. 1: 269-286). Type IV collagen is composed of six homologous α chains (α1-6) that associate into heterotrimeric collagen IV protomers, with each comprising a specific chain combination of α112, α345, or α556 (see, e.g., Khoshnoodi et al. (2008) Microsc. Res. Tech. 71: 357-370). Protomers share structural similarities of a triple-helical collagenous domain with the triplet peptide sequence of Gly-X-Y (Timpl et al. (1979) Eur. J. Biochem. 95: 255-263), ending in a globular C-terminal region termed the non-collagenous 1 (NC1) domain. The N-termini are composed of a helical domain termed the 7S domain (see, e.g., Risteli et al. (1980) Eur. J. Biochem. 108: 239-250), which is also involved in protomer-protomer interactions.

Research has suggested that collagen IV influences cellular function, inferred from the positioning of basement membranes underneath epithelial layers, and data support the role of collagen IV in tissue stabilization (see, e.g., Cummings et al., supra). Posterior capsule opacification (PCO) develops as a complication in approximately 20-40% of patients in subsequent years after cataract surgery (see, e.g., Awasthi et al. (2009) Arch Ophthalmol. 127: 555-562). PCO results from proliferation and activity of residual lens epithelial cells along the posterior capsule in a response akin to wound healing (see, e.g., Awasthi et al. (2009) Arch Ophthalmol. 127: 555-562). Growth factors, such as fibroblast growth factor, transforming growth factor β, epidermal growth factor, hepatocyte growth factor, insulin-like growth factor, and interleukins IL-1 and IL-6 may also promote epithelial cell migration, (see, e.g., Awasthi et al., supra; Raj et al., supra). As discussed herein, these factors and cytokines are also produced by senescent cells as part of the SASP. In contrast, in vitro studies show that collagen IV promotes adherence of lens epithelial cells (see, e.g., Olivero et al. (1993) Invest. Ophthalmol. Vis. Sci. 34: 2825-2834). Adhesion of the collagen IV, fibronectin, and laminin to the intraocular lens inhibits cell migration and may reduce the risk of PCO (see, e.g., Raj et al. (2007) Int. J. Biomed. Sci. 3: 237-250).

Without wishing to be bound by any particular theory, selective killing of senescent cells by DGLA, and/or GLA, and/or a D5D inhibitor alone or in combination with one or more additional senolytic agents may slow or impede (delay, inhibit, retard) the disorganization of the type IV collagen network. Removal of senescent cells and thereby removal of the inflammatory effects of SASP may decrease or inhibit epithelial cell migration and may also delay (suppress) the onset of presbyopia or decrease or slow the progressive severity of the condition (such as slow the advancement from mild to moderate or moderate to severe). DGLA, and/or GLA, and/or a D5D inhibitor alone or in combination with one or more additional senolytic agents may also be useful post-cataract surgery to reduce the likelihood of occurrence of PCO.

While no direct evidence for the involvement of cellular senescence with the development of cataracts has been obtained from human studies, BubR1 hypomorphic mice develop posterior subcapsular cataracts bilaterally early in life, suggesting that senescence may play a role (see, e.g., Baker et al. (2008) Nat. Cell Biol. 10: 825-836). Cataracts are a clouding of the lens of an eye, causing blurred vision, and if left untreated can result in blindness. Surgery is effective and routinely performed to remove cataracts. Administration of DGLA, and/or GLA, and/or a D5D inhibitor alone or in combination with one or more additional senolytic agents may result in decreasing the likelihood of occurrence of a cataract or may slow or inhibit progression of a cataract. The presence and severity of a cataract can be monitored by eye exams using methods routinely performed by a person skilled in the ophthalmology art.

In certain embodiments, DGLA, and/or GLA, and/or a D5D inhibitor alone or in combination with one or more additional senolytic agents may be administered to a subject who is at risk of developing presbyopia, cataracts, or macular degeneration. Treatment with DGLA, and/or GLA, and/or a D5D inhibitor alone or in combination with one or more additional senolytic agents may be initiated when a human subject is at least 40 years of age to delay or inhibit onset or development of cataracts, presbyopia, and macular degeneration. Because almost all humans develop presbyopia, in certain embodiments, DGLA, and/or GLA, and/or a D5D inhibitor alone or in combination with one or more additional senolytic agents may be administered in a manner as described herein to a human subject after the subject reaches the age of 40 to delay or inhibit onset or development of presbyopia.

In certain embodiments, the senescence associated disease or disorder is glaucoma. Glaucoma is a broad term used to describe a group of diseases that causes visual field loss, often without any other prevailing symptoms. The lack of symptoms often leads to a delayed diagnosis of glaucoma until the terminal stages of the disease. Even if subjects afflicted with glaucoma do not become blind, their vision is often severely impaired. Normally, clear fluid flows into and out of the front part of the eye, known as the anterior chamber. In individuals who have open/wide-angle glaucoma, this fluid drains too slowly, leading to increased pressure within the eye. If left untreated, this high pressure subsequently damages the optic nerve and can lead to complete blindness. The loss of peripheral vision is caused by the death of ganglion cells in the retina. Ganglion cells are a specific type of projection neuron that connects the eye to the brain. When the cellular network required for the outflow of fluid was subjected to SA-β-Gal staining, a fourfold increase in senescence has been observed in glaucoma patients (see, e.g., Liton et al. (2005) Exp. Gerontol. 40: 745-748).

For monitoring the effect of a therapy on inhibiting progression of glaucoma, standard automated perimetry (visual field test) is the most widely used technique. In addition, several algorithms for progression detection have been developed (see, e.g., Wesselink et al. (2009) Arch Ophthalmol. 127(3): 270-274, and references therein). Additional methods include gonioscopy (examines the trabecular meshwork and the angle where fluid drains out of the eye); imaging technology, for example scanning laser tomography (e.g., HRT3), laser polarimetry (e.g., GDX), and ocular coherence tomography); ophthalmoscopy; and pachymeter measurements that determine central corneal thickness.

### Metabolic Disease or Disorder.

Senescence-associated diseases or disorders treatable by administering DGLA, and/or GLA, and/or a D5D inhibitor alone or in combination with one or more additional senolytic agents include metabolic diseases or disorders. Such senescent cell associated diseases and disorders include diabetes, metabolic syndrome, diabetic ulcers, and obesity.

Diabetes is characterized by high levels of blood glucose caused by defects in insulin production, insulin action, or both. The great majority (90 to 95%) of all diagnosed cases of diabetes in adults are type 2 diabetes, characterized by the gradual loss of insulin production by the pancreas. Diabetes is the leading cause of kidney failure, nontraumatic lower-limb amputations, and new cases of blindness among adults in the U.S. Diabetes is a major cause of heart disease and stroke and is the seventh leading cause of death in the U.S. (see, e.g., Centers for Disease Control and Prevention, National diabetes fact sheet: national estimates and general information on diabetes and pre-diabetes in the United States, 2011 (“Diabetes fact sheet”)). In certain embodiments, DGLA, and/or GLA, and/or a D5D inhibitor alone or in combination with one or more additional senolytic agents may be used for treating type 2 diabetes, particularly age-, diet- and obesity-associated type 2 diabetes.

Involvement of senescent cells in metabolic disease, such as obesity and type 2 diabetes, has been suggested as a response to injury or metabolic dysfunction (see, e.g., Tchkonia et al. (2010) Aging Cell, 9: 667-684). Fat tissue from obese mice showed induction of the senescence markers SA-β-Gal, p53, and p21 (see, e.g., Tchkonia et al., supra; Minamino et al. (2009) Nat. Med. 15: 1082-1087). A concomitant up-regulation of pro-inflammatory cytokines, such as tumor necrosis factor-α and Ccl2/MCP1, was observed in the same fat tissue (see, e.g., Minamino et al., supra). Induction of senescent cells in obesity potentially has clinical implications because pro-inflammatory SASP components are also suggested to contribute to type 2 diabetes (see, e.g., Tchkonia et al., supra). A similar pattern of up-regulation of senescence markers and SASP components are associated with diabetes, both in mice and in humans (see, e.g., Minamino et al., supra). Accordingly, the methods described herein that comprise administering DGLA, and/or GLA, and/or a D5D inhibitor alone or in combination with one or more additional senolytic agents may be useful for treatment or prophylaxis of type 2 diabetes, as well as obesity and metabolic syndrome. Without wishing to be bound by theory, contact of senescent pre-adipocytes with DGLA, and/or GLA, and/or a D5D inhibitor alone or in combination with one or more additional senolytic agents thereby killing the senescent pre-adipocytes may provide clinical and health benefit to a person who has any one of diabetes, obesity, or metabolic syndrome.

Subjects suffering from type 2 diabetes can be identified using standard diagnostic methods known in the art for type 2 diabetes. Generally, diagnosis of type 2 diabetes is based on symptoms (e.g., increased thirst and frequent urination, increased hunger, weight loss, fatigue, blurred vision, slow-healing sores or frequent infections, and/or areas of darkened skin), medical history, and/or physical examination of a patient. Subjects at risk of developing type 2 diabetes include those who have a family history of type 2 diabetes and those who have other risk factors such as excess weight, fat distribution, inactivity, race, age, prediabetes, and/or gestational diabetes.

The effectiveness of DGLA, and/or GLA, and/or a D5D inhibitor alone or in combination with one or more additional senolytic agents can readily be determined by a person skilled in the medical and clinical arts. One or any combination of diagnostic methods, including physical examination, assessment and monitoring of clinical symptoms, and performance of analytical tests and methods, such as those described herein, may be used for monitoring the health status of the subject. A subject who is receiving DGLA, and/or GLA, and/or a D5D inhibitor alone or in combination with one or more additional senolytic agents for treatment or prophylaxis of diabetes can be monitored, for example, by assaying glucose and insulin tolerance, energy expenditure, body composition, fat tissue, skeletal muscle, and liver inflammation, and/or lipotoxicity (muscle and liver lipid by imaging in vivo and muscle, liver, bone marrow, and pancreatic β-cell lipid accumulation and inflammation by histology). Other characteristic features or phenotypes of type 2 diabetes are known and can be assayed as described herein and by using other methods and techniques known and routinely practiced in the art.

Obesity and obesity-related disorders are used to refer to conditions of subjects who have a body mass that is measurably greater than ideal for their height and frame. Body Mass Index (BMI) is a measurement tool used to determine excess body weight and is calculated from the height and weight of a subject. A human is considered overweight when the person has a BMI of 25-29; a person is considered obese when the person has a BMI of 30-39, and a person is considered severely obese when the person has a BMI of ≥40. Accordingly, the terms obesity and obesity-related refer to human subjects with body mass index values of greater than 30, greater than 35, or greater than 40. A category of obesity not captured by BMI is called “abdominal obesity” in the art, which relates to the extra fat found around a subject's middle, which is an important factor in health, even independent of BMI. The simplest and most often used measure of abdominal obesity is waist size. Generally abdominal obesity in women is defined as a waist size 35 inches or higher, and in men as a waist size of 40 inches or higher. More complex methods for determining obesity require specialized equipment, such as magnetic resonance imaging or dual energy X-ray absorption metry machines.

A condition or disorder associated with diabetes and senescence is a diabetic ulcer (i.e., diabetic wound). An ulcer is a breakdown in the skin, which may extend to involve the subcutaneous tissue or even muscle or bone. These lesions occur, particularly, on the lower extremities. Patients with diabetic venous ulcer exhibit elevated presence of cellular senescence at sites of chronic wounds (see, e.g., Stanley et al. (2001) J. Vas. Surg. 33: 1206-1211). Chronic inflammation is also observed at sites of chronic wounds, such as diabetic ulcers (see, e.g., Goren et al. (2006) Am. J. Pathol. 7 168: 65-77; Seitz et al. (2010) Exp. Diabetes Res. 2010: 476969), suggesting that the proinflammatory cytokine phenotype of senescent cells has a role in the pathology.

Subjects who have type 2 diabetes or who are at risk of developing type 2 diabetes may have metabolic syndrome. Metabolic syndrome in humans is typically associated with obesity and characterized by one or more of cardiovascular disease, liver steatosis, hyperlipidemia, diabetes, and insulin resistance. A subject with metabolic syndrome may present with a cluster of metabolic disorders or abnormalities that may include, for example, one or more of hypertension, type-2 diabetes, hyperlipidemia, dyslipidemia (e.g., hypertriglyceridemia, hypercholesterolemia), insulin resistance, liver steatosis (steatohepatitis), hypertension, atherosclerosis, and other metabolic disorders.

### Renal Dysfunction

Nephrological pathologies, such as glomerular disease, arise in the elderly. Glomerulonephritis is characterized by inflammation of the kidney and by the expression of two proteins, IL1α and IL1β (see, e.g., Niemir et al. (1997) Kidney Int. 52: 393-403). IL1α and IL1β are considered master regulators of SASP (see, e.g., Coppe et al. (2008) PLoS. Biol. 6: 2853-68). Glomerular disease is associated with elevated presence of senescent cells, especially in fibrotic kidneys (see, e.g., Sis et al. (2007) Kidney Int. 71: 218-226) as well as diabetic kidney disease.

### Dermatological Disease or Disorder.

Senescence-associated diseases or disorders treatable by administering DGLA, and/or GLA, and/or a D5D inhibitor alone or in combination with one or more additional senolytic agents include dermatological diseases or disorders. Such senescent cell associated diseases and disorders include psoriasis and eczema, which are also inflammatory diseases and are discussed in greater detail above. Other dermatological diseases and disorders that are associated with senescence include rhytides (wrinkles due to aging), pruritis (linked to diabetes and aging), dysesthesia (chemotherapy side effect that is linked to diabetes and multiple sclerosis), psoriasis (as noted) and other papulosquamous disorders, for example, erythroderma, lichen planus, and lichenoid dermatosis, atopic dermatitis (a form of eczema and associated with inflammation), eczematous eruptions (often observed in aging patients and linked to side effects of certain drugs). Other dermatological diseases and disorders associated with senescence include eosinophilic dermatosis (linked to certain kinds of hemotologic cancers), reactive neutrophilic dermatosis (associated with underlying diseases such as inflammatory bowel syndrome), pemphigus (an autoimmune disease in which autoantibodies form against desmoglein), pemphigoid and other immunobullous dermatosis (autoimmune blistering of skin), fibrohistocytic proliferations of skin, which is linked to aging, and cutaneous lymphomas that are more common in older populations. Another dermatological disease that may be treatable according to the methods described herein includes cutaneous lupus, which is a symptom of lupus erythematosus. Late onset lupus may be linked to decreased (i.e., reduced) function of T-cell and B-cells and cytokines (immunosenescence) associated with aging. Yet another dermatologica disease that may be treatable by methods describe herein includes squamous cell carcinoma (Alimirah et al., (2020) Cancer Res, in press).

### D5D Inhibitors

As noted above, in certain embodiments the methods described herein involve administering to a subject an effective amount of one or more agents selected from the group consisting of dihomo-gamma-linolenic acid (DGLA), and/or gamma-linolenic acid (GLA), and/or a delta-5-desaturase inhibitor (D5D inhibitor). In certain embodiments the D5D inhibitor is administered in conjunction with DGLA. In certain embodiments the D5D inhibitor is administered in conjunction with GLA. In certain embodiments DGLA, GLA, and a D5D inhibitor are all administered.

D5D inhibitors are well known to those of skill in the art. Illustrative examples of D5D inhibitors are described in U.S. Patent Publication No: 2019/0070193. The D5d inhibitors described therein include, but are not limited to iminodibenzyl, iminostilbene, and derivatives thereof as shown in Table 1.

TABLE 1
Illustrative D5D inhibitors described in U.S. Patent Publication No:
2019/0070193.

| Compound | Structure |
|---|---|
| Imino- dibenzyl | 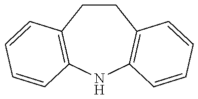 [SMILES: c1ccc2c(c1)CCc1ccccc1N2] |
| Imino- stilbene | 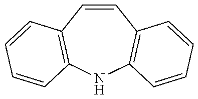 [SMILES: C1=Cc2ccccc2Nc2ccccc21] |
| 1a | 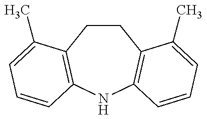 [SMILES: Cc1cccc2c1CCc1c(C)cccc1N2] |
| 3a | 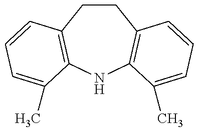 [SMILES: Cc1cccc2c1Nc1c(C)cccc1CC2] |
| 1b | 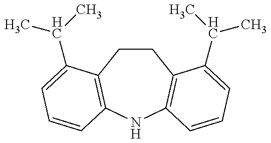 [SMILES: CC(C)c1cccc2c1CCc1c(cccc1C(C)C)N2] |
| 3b | 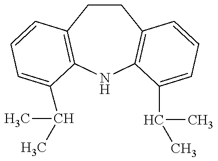 [SMILES: CC(C)c1cccc2c1Nc1c(cccc1C(C)C)CC2] |
| 1d | 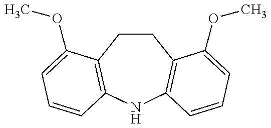 |
| 1e | 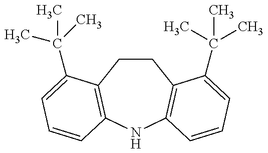 [SMILES: CC(C)(C)c1cccc2c1CCc1c(cccc1C(C)(C)C)N2] |
| 1f | 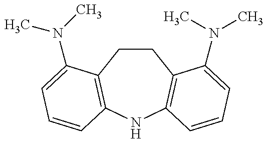 |
| 2e | 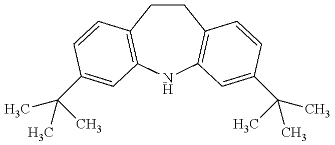 [SMILES: CC(C)(C)c1ccc2c(c1)Nc1cc(C(C)(C)C)ccc1CC2] |
| 3e | 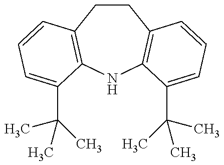 [SMILES: CC(C)(C)c1cccc2c1Nc1c(cccc1C(C)(C)C)CC2] |
| 2f | 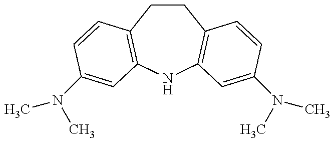 |
| 3f | 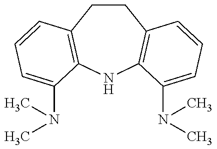 [SMILES: CN(C)c1cccc2c1Nc1c(cccc1N(C)C)CC2] |

Still other illustrative and non-limiting examples of D5D inhibitors are described in PCT Publication Nos: WO2008/089307, and WO2008/089310. These include but are not limited to compounds 1-354 shows in Table 2.

TABLE 2
Illustrative D5D inhibitors described in PCT Publication
Nos: WO2008/089307, and WO2008/089310.

| Cmpd | Name |
|---|---|
| 1 | 6-(furan-3-yl)-N-phenylquinazolin-4-amine |
| 2 | 7-chloro-N-(3- |
| | chlorophenyl)quinazolin-4-amine |
| 3 | l-(3-chlorophenyl)-3-(3,5- |
| | dimethylphenyl)urea |
| 4 | N-(3-chlorophenyl)-6- |
| | methoxyquinazolin-4-amine |
| 5 | 4-(3-chlorophenylamino)quinazolin-6-ol |
| 6 | tert-butyl 5-(3-chlorophenylamino)-3,4- |
| | dihydroisoquinoline-2(1H)-carboxylate |
| 7 | 4-(3-chlorophenylamino)quinazolin-6- |
| | yl acetate |
| 8 | 5-bromo-N-(3-chlorophenyl)-1- |
| | methyl-1H-indazol-3-amine |
| 9 | N³-(3-chlorophenyl)-1H-indazole-3,5- |
| | diamine |
| 10 | methyl 2-(3-(3-chlorophenylamino)- |
| | 1H-indazol-5-ylamino)acetate |
| 11 | N-(3-(3-chlorophenylamino)-1H- |
| | indazol-7-yl)acetamide |
| 12 | N-(3-chlorophenyl)-7- |
| | (trifluoromethyl)-1H-indazol-3-amine |
| 13 | N-(3-chlorophenyl)-6- |
| | fluoroquinazolin-4-amine |
| 14 | (3-(3- |
| | chlorophenylamino)benzo[b]thiophen- |
| | 2-yl)methanol |
| 15 | N-(3-chlorophenyl)-7-ethyl-1 H- |
| | indazol-3-amine |
| 16 | 5-bromo-N,l-bis(3-chlorophenyl)-1H- |
| | indazol-3-amine |
| 17 | 5-bromo-N-(3-chlorophenyl)-1H- |
| | indazol-3-amine |
| 18 | N-(3-chlorophenyl)-7-nitro-1H- |
| | indazol-3-amine |
| 19 | 7-bromo-N-(3-chlorophenyl)-1H- |
| | indazol-3-amine |
| 20 | 3-(3-chlorophenylamino)-1H-indazol-5-ol |
| 21 | (Z)-7-(but-2-en-2-yl)-N-(3- |
| | chlorophenyl)-1H-indazol-3-amine |
| 22 | N-(3-chlorophenyl)benzo[d]isothiazol- |
| | 3-amine |
| 23 | N-(3-chlorophenyl)isoquinolin-l-amine |
| 24 | N-(3-chlorophenyl)quinazolin-4-amine |
| 25 | N-(3-chlorophenyl)-5-methyl-7H- |
| | pyrrolo[2,3-d]pyrimidin-4-amine |
| 26 | N-(3-chlorophenyl)-7H-pyrrolo[2,3- |
| | d]pyrimidin-4-amine |
| 27 | 4-chloro-N-(3- |
| | chlorophenyl)phthalazin-1-amine |
| 28 | N³-(3-chlorophenyl)-1H-indazole-3,7- |
| | diamine |
| 29 | N-(3-chlorophenyl)-5-methyl-1H- |
| | indazol-3-amine |
| 30 | N⁶ < 3-cMorophenyl)-N⁶-methyl- |
| | N⁶ < (R)4-phenylethyl)-23-dihydro4H- |
| | indene-l,6-diamine |
| 31 | N-(3-chlorophenyl)-5-methoxy-l H- |
| | indazol-3-amine |
| 32 | 2-amino-N-m-tolylbenzamide |
| 33 | 5-amino-N-(3-chlorophenyl)-2- |
| | hydroxybenzamide |
| 34 | N-(3-chlorophenyl)naphthalen-1-amine |
| 35 | N-(3-chlorophenyl)isoquinolin-4-amine |
| 36 | N-(3-chlorophenyl)-8- |
| | fluoroquinazolin-4-amine |
| 37 | N\hat{ }N\hat{ }-bisCS-chlorophenyO-l\hat{ }′\hat{ }\hat{ }′- |
| | tetrahydro-1HJH-S\hat{ }′-biindene-S\hat{ }′- |
| | diamine |
| 38 | N⁶-(3-chlorophenyl)-N⁶-(2- |
| | methoxyethyl)-N⁶-methyl-2,3- |
| | dihydro-1H-indene-l,6-diamine |
| 39 | N-(3-chlorophenyl)-5,7- |
| | difluoroquinazolin-4-amine |
| 40 | N-(3-chlorophenyl)-6,7- |
| | difluoroquinazolin-4-amine |
| 41 | N-(3-chlorophenyl)thieno[2,3- |
| | d]pyrimidin-4-amine |
| 42 | N⁶-(3-chlorophenyl)isoquinoline-l,3- |
| | diamine |
| 43 | N-(3-chlorophenyl)thieno [3,2- |
| | d]pyrimidin-4-amine |
| 44 | N⁶-(3-chlorophenyl)-N⁶-(4- |
| | fluorobenzyl)-N⁶-methyl-2,3-dihydro- |
| | 1H-indene-l,6-diamine |
| 45 | N⁶-(3-chlorophenyl)-N⁶-(3- |
| | methoxybenzyl)-N⁶-methyl-2,3- |
| | dihydro-1H-indene-l,6-diamine |
| 46 | N-(3-chlorophenyl)-2-methoxy-7H- |
| | pyrrolo[2,3-d]pyrimidin-4-amine |
| 47 | N-(3-chlorophenyl)fluro[3,2-c]pyridin- |
| | 4-amine |
| 48 | N-(3-chlorophenyl)furo[3,2-c]pyridin- |
| | 4-amine |
| 49 | N⁶-(3-chlorophenyl)-N⁶-(4- |
| | methoxyphenyl)-N⁶-methyl-2,3- |
| | dihydro-1H-indene-l,6-diamine |
| 50 | N⁶-(4-methoxyphenyl)-N⁶-(3-((4- |
| | methoxyphenyl)(methyl)amino)phenyl)- |
| | N⁶-methyl-2,3-dihydro-1H-indene- |
| | 1,6-diamine |
| 51 | (R)-6-chloro-N-(3-chlorophenyl)-2,3- |
| | dihydro-1H-inden-l-amine |
| 52 | N⁶-(3-chlorophenyl)-N⁶-methyl-N⁶-(4- |
| | morpholinobenzyl)-2,3-dihydro-1H- |
| | indene-l,6-diamine |
| 53 | N⁶-(3-chlorophenyl)-N⁶-(2,4- |
| | dimethoxybenzyl)-N⁶-methyl-2,3- |
| | dihydro-1H-indene-1,6-diamine |
| 54 | N⁶-(3-chlorophenyl)-N⁶-(2- |
| | (dimethylamino)ethyl)-N⁶-methyl-2,3- |
| | dihydro-1H-indene-1,6-diamine |
| 55 | N-(3-chlorophenyl)-7-methyl-7H- |
| | pyrrolo[2,3-d]pyrimidin-4-amine |
| 56 | N-(3-chlorophenyl)imidazo[l,2- |
| | a]pyridin-8-amine |
| 57 | N-(3-chlorophenyl)cinnolin-4-amine |
| 58 | 4-(3-chlorophenylamino)thieno[3,2- |
| | c]pyridine-2-carbonitrile |
| 59 | N⁶-(3-chlorophenyl)-N⁶-(4- |
| | methoxybenzyl)-2,3-dihydro-1H- |
| | indene-l,6-diamine |
| 60 | N⁶-(3-chlorophenyl)-N⁶-methyl-N⁶-(4- |
| | (trifluoromethyl)benzyl)-2,3-dihydro- |
| | 1H-indene-1,6-diamine |
| 61 | N⁶-benzyl-N⁶-(3-chlorophenyl)-N⁶- |
| | methyl-2,3-dihydro-1H-indene-l,6- |
| | diamine |
| 62 | N-(3-bromophenyl)quinolin-4-amine |
| 63 | 6-chloro-N-(3-chlorophenyl)-2,3- |
| | dihydro-1H-inden-1-amine |
| 64 | N-(3-chlorophenyl)-6-methyl-2,3- |
| | dihydro-1H-inden-1-amine |
| 65 | 3-(3-chlorophenylamino)-2,3-dihydro- |
| | 1H-indene-5-carbonitrile |
| 66 | N⁶-(3-chlorophenyl)-N⁶-(2- |
| | ethoxyethyl)-2,3-dihydro-1H-indene- |
| | l,6-diamine |
| 67 | N-(3-chlorophenyl)-6-methoxy-2,3- |
| | dihydro-1H-inden-1-amine |
| 68 | 6-chloro-N-(3- |
| | chlorophenyl)quinazolin-4-amine |
| 69 | N-(3-chlorophenyl)phthalazin-1-amine |
| 70 | N-(3-chlorophenyl)-4- |
| | methylnaphthalen-1-amine |
| 71 | N-(3-chlorophenyl)-6-methoxy-2,3- |
| | dihydro-1H-inden-1-amine |
| 72 | 2-bromo-N-(3- |
| | chlorophenyl)thieno[3,2-c]pyridin-4- |
| | amine |
| 73 | N-(3-fluorophenyl)-2,3-dihydro-1H- |
| | inden-1-amine |
| 74 | (2R)-l-(3-chlorophenylamino)-2,3- |
| | dihydro-1H-inden-2-ol |
| 75 | N⁶ < 3-cMorophenyl)-N⁶-methyl- |
| | N⁶ < (R)4-phenylethyl)-23-dihydro4H- |
| | indene-l,6-diamine |
| 76 | N-(3-chlorophenyl)-2,3-dihydro-1H- |
| | inden-4-amine |
| 77 | N-(l-(3- |
| | chlorophenylamino)isoquinolin-3- |
| | yl)acetamide |
| 78 | N-(3-chlorophenyl)-5,6,7,8- |
| | tetrahydronaphthalen-l-amine |
| 79 | 2-chloro-N-(3- |
| | chlorophenyl)thieno[3,2-c]pyridin-4- |
| | amine |
| 80 | N-(3-chlorophenyl)-2-methylfuro[3,2- |
| | c]pyridin-4-amine |
| 81 | N-(3-chlorophenyl)-6-fluoro-2,3- |
| | dihydro-1H-inden-1-amine |
| 82 | 6-(benzyl(methyl)amino)-2,3-dihydro- |
| | 1H-inden-1-one |
| 83 | N⁶-benzyl-N⁶-(3-chlorophenyl)-N⁶- |
| | ethyl-2,3-dihydro-1H-indene-l,6- |
| | diamine |
| 84 | 3-amino-N-(3-chlorophenyl)-2- |
| | naphthamide |
| 85 | 3-chloro-N-(4-fluoro-1H-indazol-3- |
| | yl)benzamide |
| 86 | N-(2,3-dihydro-1H-inden-4-yl)-7H- |
| | pyrrolo[2,3-d]pyrimidin-4-amine |
| 87 | N-(3-chlorophenyl)-N-(6- |
| | (trifluoromethyl)quinolin-4- |
| | yl)acetamide |
| 88 | 4-(3-chlorophenylamino)-6- |
| | (trifluoromethyl)quinoline 1-oxide |
| 89 | N⁶-(3-chlorophenyl)-N⁶-(2-fluoro-4- |
| | methoxybenzyl)-N⁶-methyl-2,3- |
| | dihydro-1H-indene-l,6-diamine |
| 90 | N⁶-(3-chlorophenyl)-N⁶-(3-fluoro-4- |
| | methoxybenzyl)-N⁶-methyl-2,3- |
| | dihydro-1H-indene-1,6-diamine |
| 91 | 7-chloro-N-(3-chlorophenyl)-1H- |
| | indazol-3-amine |
| 92 | 5-chloro-N-(3-chlorophenyl)-1H- |
| | indazol-3-amine |
| 93 | N-(3-chlorophenyl)-5,7-difluoro-1H- |
| | indazol-3-amine |
| 94 | 5-fluoro-N-(3-fluorophenyl)-1H- |
| | indazol-3-amine |
| 95 | N-(3-fluorophenyl)-1H-indazol-3- |
| | amine |
| 96 | 5-fluoro-N-phenyl-1H-indazol-3- |
| | amine |
| 97 | 3-(3-chlorophenylamino)benzofuran- |
| | 2-carboxamide |
| 98 | 5-chloro-N-(3-chlorophenyl)-1- |
| | methyl-1H-indazol-3-amine |
| 99 | 5-chloro-N-(3-fluorophenyl)-1- |
| | methyl-1H-indazol-3-amine |
| 100 | N-(3-chlorophenyl)benzo[d]isoxazol- |
| | 3-amine |
| 101 | N-(3-chlorophenyl)-l H-indazol-3- |
| | amine |
| 102 | N-(3-chlorophenyl)-l-methyl-1H- |
| | pyrazolo[3,4-b]pyridin-3-amine |
| 103 | N-phenyl-1H-indazol-3-amine |
| 104 | N,1-diphenyl-l H-indazol-3-amine |
| 105 | N-(3-chlorophenyl)-5-fluoro-1H- |
| | indazol-3-amine |
| 106 | N-(3-chlorophenyl)-5-methoxy-1- |
| | methyl-1H-indazol-3-amine |
| 107 | N-(3-fluorophenyl)-5-methoxy-l- |
| | methyl-1H-indazol-3-amine |
| 108 | 4-chloro-N-(5-methoxy-l H-indazol-3- |
| | yl)benzamide |
| 109 | N-(3-chlorophenyl)-6- |
| | phenylquinazolin-4-amine |
| 110 | N-(3-chlorophenyl)-6-(4- |
| | fluorophenyl)quinazolin-4-amine |
| 111 | N-(3-chlorophenyl)-6-(2,3- |
| | difluorophenyl)quinazolin-4-amine |
| 112 | methyl 3-(4-(4-(3- |
| | chlorophenylamino)quinazolin-6- |
| | yl)phenyl)propanoate |
| 113 | N-(3-chlorophenyl)-6-(thiophen-3- |
| | yl)quinazolin-4-amine |
| 114 | N-(3-chlorophenyl)-8-methyl-9H- |
| | purin-6-amine |
| 115 | 6-(6-bromopyridin-3-yl)-N-(3- |
| | chlorophenyl)quinazolin-4-amine |
| 116 | 4-(4-(3- |
| | chlorophenylamino)quinazolin-6- |
| | yl)benzoic acid |
| 117 | (3-chlorophenyl)(5-methoxy-1H- |
| | indazol-3-yl)methanone |
| 118 | 4-chloro-N-(5-methoxy-l H-indazol-3- |
| | yl)benzenesulfonamide |
| 119 | N-(3-chlorophenyl)-6-(6- |
| | chloropyridin-3-yl)quinazolin-4-amine |
| 120 | N-(3-chlorophenyl)-6-(3- |
| | fluorobiphenyl-4-yl)quinazolin-4- |
| | amine |
| 121 | tert-butyl 4-(4-(3- |
| | chlorophenylamino)quinazolin-6- |
| | yl)phenylcarbamate N-(3- |
| | chlorophenyl)-6-ethylquinazolin-4- |
| | amine |
| 122 | N-(3-chlorophenyl)-6-(4- |
| | chlorophenyl)quinazolin-4-amine |
| 123 | 6-(benzo[d] [1,3]dioxol-5-yl)-N-(3- |
| | chlorophenyl)quinazolin-4-amine |
| 124 | N-(3-chlorophenyl)-6-(2-fluoro-3- |
| | methoxyphenyl)quinazolin-4-amine |
| 125 | N-(3-chlorophenyl)-6-(4- |
| | (morpholinosulfonyl)phenyl)quinazolin- |
| | 4-amine |
| 126 | l-(4-(4-(3- |
| | chlorophenylamino)quinazolin-6- |
| | yl)benzoyl)piperidin-4-one |
| 127 | 4-(4-(3- |
| | chlorophenylamino)quinazolin-6- |
| | yl)phenol |
| 128 | N-(3-chlorophenyl)-6-(3- |
| | fluorophenyl)quinazolin-4-amine |
| 129 | 6-bromo-N-(3- |
| | chlorophenyl)quinazolin-4-amine |
| 130 | N-(3-chlorophenyl)-6-(2- |
| | fluorophenyl)quinazolin-4-amine |
| 131 | N-(3-chlorophenyl)-6-(thiophen-2- |
| | yl)quinazolin-4-amine |
| 132 | N-(3-chlorophenyl)-6-(3,5- |
| | dimethylisoxazol-4-yl)quinazolin-4- |
| | amine |
| 133 | N-(3-chlorophenyl)-6- |
| | cyclopropylquinazolin-4-amine |
| 134 | N-(3-chlorophenyl)-1H-pyrazolo[3,4- |
| | d]pyrimidin-4-amine |
| 135 | N-(3-chlorophenyl)-6-(5- |
| | methoxypyridin-3-yl)quinazolin-4- |
| | amine |
| 136 | 4-(4-(3- |
| | chlorophenylamino)quinazolin-6- |
| | yl)benzonitrile |
| 137 | N-(3-chlorophenyl)-3-methyl-1H- |
| | pyrazolo [3,4-d]pyrimidin-4-amine |
| 138 | N²,N⁴-bis(3-chlorophenyl)pyrido[2,3- |
| | d]pyrimidine-2,4-diamine |
| 139 | N,1-bis(4-methoxybenzyl)-5-nitro- |
| | 1H-indazol-3-amine |
| 140 | N-(5-methoxy-l H-indazol-3- |
| | yl)thiophene-2-sulfonamide |
| 141 | N-(3-chlorophenyl)-6-(6- |
| | fluoropyridin-3-yl)quinazolin-4-amine |
| 142 | N-(3-chlorophenyl)-6- |
| | methylthieno[2,3-d]pyrimidin-4-amine |
| 143 | N-(3-chlorophenyl)-6-(5- |
| | methylthiophen-2-yl)quinazolin-4- |
| | amine |
| 144 | N-(3-chlorophenyl)-6- |
| | methylquinazolin-4-amine |
| 145 | 6-(5-bromothiophen-2-yl)-N-(3- |
| | chlorophenyl)quinazolin-4-amine |
| 146 | N-(3-chlorophenyl)-7- |
| | methylthieno[3,2-d]pyrimidin-4-amine |
| 147 | N-(3-chlorophenyl)-6- |
| | cyclohexenylquinazolin-4-amine |
| 148 | N-(3-chlorophenyl)-6-(1H-pyrrol-2- |
| | yl)quinazolin-4-amine |
| 149 | N-(3-chlorophenyl)-1H-pyrrolo[3,2- |
| | c]pyridin-4-amine |
| 150 | N-(3-chlorophenyl)-5-nitro-1H- |
| | indazol-3-amine |
| 151 | 2-fluoro-N-(5-methoxy-l H-indazol-3- |
| | yl)benzenesulfonamide |
| 152 | N-(3-chlorophenyl)quinolin-4-amine |
| 153 | 8-chloro-N-(3-chlorophenyl)quinolin- |
| | 4-amine |
| 154 | N-(3-chlorophenyl)-6-fluoroquinolin- |
| | 4-amine |
| 155 | N-(5-chloro-2-fluorophenyl)-7H- |
| | pyrrolo[2,3-d]pyrimidin-4-amine |
| 156 | N-(3-chlorophenyl)-6- |
| | isopropylthieno[2,3-d]pyrimidin-4- |
| | amine |
| 157 | N-(3-chlorophenyl)-2,3-dihydro-1H- |
| | inden-l-amine |
| 158 | N-(3-chlorophenyl)-2-ethylfuro[3,2- |
| | c]pyridin-4-amine |
| 159 | 6-bromo-N-(3-chlorophenyl)-2,3- |
| | dihydro-1H-inden-1-amine |
| 160 | N-(3-chlorophenyl)-6-morpholino-2,3- |
| | dihydro-1H-inden-l-amine |
| 161 | N⁶-(3-chlorophenyl)-N⁶-(2- |
| | (dimethylamino)ethyl)-2,3-dihydro- |
| | 1H-indene-l,6-diamine |
| 162 | N-(3-chlorophenyl)-2-methylquinolin- |
| | 4-amine |
| 163 | N-(3-chlorophenyl)-4- |
| | fluoronaphthalen-1-amine |
| 164 | 3-chloro-N-(3- |
| | chlorophenyl)isoquinolin-1-amine |
| 165 | N-(3-chlorophenyl)-6- |
| | (trifluoromethyl)quinolin-4-amine |
| 166 | N⁶-(3-chlorophenyl)-N⁶-(4- |
| | methoxybenzyl)-N⁶-methyl-2,3- |
| | dihydro-1H-indene-l,6-diamine |
| 167 | N-(3-chlorophenyl)-5-methoxy-l-(4- |
| | methoxybenzyl)-1H-indazol-3-amine |
| 168 | N-(3-chlorophenyl)-5-methoxy-l-(4- |
| | methoxybenzyl)-1H-indazol-3-amine |
| 169 | 6-bromo-N-(3-chlorophenyl)-2,3- |
| | dihydro-1H-inden-l-amine |
| 170 | N⁶-(3-chlorophenyl)-N⁶-methyl-N⁶- |
| | phenethyl-2,3-dihydro-1H-indene-l,6- |
| | diamine |
| 171 | N⁶-(3-chlorophenyl)-N⁶-(4- |
| | ethylbenzyl)-N⁶-methyl-2,3-dihydro- |
| | 1H-indene-l,6-diamine |
| 172 | N⁶-benzyl-N⁶-(3-chlorophenyl)-N⁶- |
| | methyl-2,3-dihydro-1H-indene-l,6- |
| | diamine |
| 173 | N-(3-chlorophenyl)-l-methyl-1H- |
| | indole-3-carboxamide |
| 174 | 5-(difluoromethyl)-3-(naphthalen-2- |
| | yl)-1H-pyrazole |
| 175 | N-(3-chlorophenyl)-2,3-dihydro-1H- |
| | inden-l-amine |
| 176 | N-(3-chlorophenyl)-6,8- |
| | difluoroquinolin-4-amine |
| 177 | N-(3-fluorophenyl)-7H-pyrrolo[2,3- |
| | d]pyrimidin-4-amine |
| 178 | N-(3-chlorophenyl)-5H- |
| | pyrrolo[3,2-d]pyrimidin-4-amine |
| 179 | N⁶-(3-chlorophenyl)-N⁶-(4- |
| | (dimethylamino)benzyl)-N⁶- |
| | methyl-2,3-dihydro-1H-indene- |
| | 1,6-diamine |
| 180 | N⁶-(4-chlorobenzyl)-N⁶-(3- |
| | chlorophenyl)-N⁶-methyl-2,3- |
| | dihydro-1H-indene-l,6-diamine |
| 181 | N⁶-butyl-N⁶-(3-chlorophenyl)-N⁶- |
| | methyl-2,3-dihydro-1H-indene- |
| | l,6-diamine |
| 182 | N⁶-(3-chloro-phenyl)-N⁶-methyl- |
| | N⁶-(l-methyl-piperidin-4-yl)- |
| | indan-l,6-diamine |
| 183 | N³-(3-chloro-phenyl)-N⁵-indan-1- |
| | yl-1-methyl-1H-indazole-3,5- |
| | diamine |
| 184 | (3-chloro-phenyl)-[5-(3-methoxy- |
| | benzyloxy)-l-methyl-1H-indazol- |
| | 3-yl]-amine |
| 185 | (3-chloro-phenyl)-(6-pyrazol-1- |
| | yl-indan-1-yl)-amine |
| 186 | N³-(3-chloro-phenyl)-N⁵- |
| | isobutyl-l,N⁵-dimethyl-1H- |
| | indazole-3,5-diamine |
| 187 | N³-(3-chloro-phenyl)-N⁵-(2- |
| | methoxy-ethyl)-l,N⁵-dimethyl- |
| | 1H-indazole-3,5-diamine |
| 188 | (R)-N⁶-(3-chloro-phenyl)-N⁶-(4- |
| | methoxy-benzyl)-N⁶-methyl- |
| | indan-l,6-diamine |
| 189 | (3,5-difluoro-phenyl)-[5- |
| | methoxy-1-(4-methoxy-benzyl)- |
| | 1H-indazol-3-yl]-amine |
| 190 | (3,5-dichloro-phenyl)-[5- |
| | methoxy-l-(4-methoxy-benzyl)- |
| | 1H-indazol-3-yl]-amine |
| 191 | (3,5-difluoro-phenyl)-(5- |
| | methoxy-1H-indazol-3-yl)-amine |
| 192 | (3,5-dichloro-phenyl)-(5- |
| | methoxy-1H-indazol-3-yl)-amine |
| 193 | N³-(3-chloro-phenyl)-N⁵-(2- |
| | methoxy-ethyl)-l-methyl-1H- |
| | indazole-3,5-diamine |
| 194 | N⁶-(3-chloro-phenyl)-N⁶-methyl- |
| | N⁶-pyridin-3-ylmethyl-indan-l,6- |
| | diamine |
| 195 | N⁶-(3-chloro-phenyl)-N⁶-methyl- |
| | N⁶-pyridin-4-ylmethyl-indan-l,6- |
| | diamine |
| 196 | (3-chloro-phenyl)-(5-fluoro-l- |
| | methyl-1H-indazol-3-yl)-amine |
| 197 | (3-chloro-phenyl)-(l-methyl-5- |
| | propoxy-1H-indazol-3-yl)-amine |
| 198 | N⁶-(3-chloro-phenyl)-N⁶-methyl- |
| | N⁶-thiophen-2-ylmethyl-indan- |
| | 1,6-diamine |
| 199 | (3-chloro-phenyl)-(l-methyl-1H- |
| | indazol-3-yl)-amine |
| 200 | 4-(3-chloro-phenylamino)- |
| | chromen-2-one |
| 201 | l-[3-(3-chloro-phenylamino)-l- |
| | methyl-1H-indazol-5-yl]- |
| | ethanone |
| 202 | N³-(3-chloro-phenyl)-N⁵-(2- |
| | methoxy-ethyl)-N⁵-methyl-1H- |
| | indazole-3,5-diamine N³-(3- |
| | chloro-phenyl)-N⁵-ethyl-l,N⁵- |
| | dimethyl-1H-indazole-3,5- |
| | diamine |
| 203 | (3-Fluoro-phenyl)-(5-methoxy- |
| | 1H-indazol-3-yl)-amine |
| 204 | (2-chloro-pyridin-4-yl)-(5- |
| | methoxy-1H-indazol-3-yl)-amine |
| 205 | (5-chloro-pyridin-3-yl)-(5- |
| | methoxy-1H-indazol-3-yl)-amine |
| 206 | N⁶-(3-chloro-phenyl)-N⁶-methyl- |
| | N⁶-thiophen-3-ylmethyl-indan- |
| | l,6-diamine |
| 207 | 1-[3-(3-chloro-phenylamino)-l- |
| | methyl-1H-indazol-5-yl]-ethanol |
| 208 | (3-chloro-phenyl)-(1-methyl-5- |
| | methylsulfanyl-1H-indazol-3-yl)- |
| | amine |
| 209 | 2-[3-(3-chloro-phenylamino)-5- |
| | methoxy-indazol-l-yl]-acetamide |
| 210 | (3-chloro-phenyl)-(5- |
| | methoxymethyl-1-methyl-1H- |
| | indazol-3-yl)-amine |
| 211 | (3-chloro-phenyl)-(l-methyl-5- |
| | pyrrol-l-yl-1H-indazol-3-yl)- |
| | amine |
| 212 | (3-chloro-phenyl)-(1-methyl-5- |
| | pyrazol-1-yl-1H-indazol-3-yl)- |
| | amine |
| 213 | 3-(3-chloro-phenylamino)-l- |
| | methyl-1H-indazole-5-carboxylic |
| | acid amide |
| 214 | N³-(3-chloro-phenyl)-l,N⁵- |
| | dimethyl-1H-indazole-3,5- |
| | diamine |
| 215 | {[3-(3-chloro-phenylamino)- |
| | indan-5-yl]-methyl-amino}-acetic |
| | acid ethyl ester |
| 216 | 1-[3-(3-chloro-phenylamino)-5- |
| | methoxy-indol-1-yl]-ethanone |
| 217 | (3-chloro-phenyl)-(5-pyrazol-l-yl- |
| | 1H-indazol-3-yl)-amine |
| 218 | (2-chloro-pyrimidin-4-yl)-[5- |
| | methoxy-1-(4-methoxy-benzyl)- |
| | 1H-indazol-3-yl]-amine |
| 219 | (3-chloro-phenyl)-(5-methoxy- |
| | 1H-indol-3-yl)-amine |
| 220 | (5-chloro-benzo[d]isoxazol-3-yl)- |
| | (3-chloro-phenyl)-amine |
| 221 | [3-(3-chloro-phenylamino)-1H- |
| | indazol-5-yl]-methanol |
| 222 | (3-chloro-phenyl)-(1H- |
| | pyrazolo[4,3-b]pyridin-3-yl)- |
| | amine |
| 223 | 3-(3-chloro-phenylamino)-1H- |
| | indazole-5-carboxylic acid amide |
| 224 | (3-chloro-phenyl)-[1-methyl-5- |
| | (4-methyl-pyrazol-1-yl)-1H- |
| | indazol-3-yl]-amine |
| 225 | 3-(3-chloro-phenylamino)-1H- |
| | indazole-5-carboxylic acid |
| | methyl ester |
| 226 | indole-1-carboxylic acid (3- |
| | chloro-phenyl)-amide |
| 227 | (3-bromo-phenyl)-(5-chloro-1- |
| | methyl-1H-indazol-3-yl)-amine |
| 228 | (5-chloro-1-methyl-1H-indazol-3- |
| | yl)-(3-iodo-phenyl)-amine |
| 229 | (3-chloro-phenyl)-(1H- |
| | pyrazolo[3,4-b]pyridin-3-yl)- |
| | amine |
| 230 | 3-(3-chloro-phenylamino)-1H- |
| | indazole-5-carbonitrile |
| 231 | [5-(4-bromo-pyrazol-l-yl)-l- |
| | methyl-1H-indazol-3-yl]-(3- |
| | chloro-phenyl)-amine |
| 232 | (7-chloro-4-fluoro-l H-indazol-3- |
| | yl)-(3-chloro-phenyl)-amine |
| 233 | (5-chloro-l-methyl-1H- |
| | pyrazolo[3,4-b]pyridin-3-yl)-(3- |
| | chloro-phenyl)-amine |
| 234 | (5-chloro-4-fluoro-l H-indazol-3- |
| | yl)-(3-chloro-phenyl)-amine |
| 235 | (3-chloro-phenyl)-(5-methoxy- |
| | benzo[d]isoxazol-3-yl)-amine |
| 236 | (3-chloro-phenyl)-(1H- |
| | pyrazolo[3,4-c]pyridin-3-yl)- |
| | amine |
| 237 | [5-chloro-l-(4-methoxy-benzyl)- |
| | 1H-pyrazolo[3,4-b]pyridin-3-yl]- |
| | (3-chloro-phenyl)-amine |
| 238 | (3-chloro-phenyl)-(6-iodo- |
| | quinazolin-4-yl)-amine |
| 239 | (3-chloro-phenyl)-(5- |
| | trifluoromethoxy-1H-indazol-3- |
| | yl)-amine |
| 240 | (3-chloro-phenyl)-(5-chloro-1H- |
| | pyrazolo[3,4-b]pyridin-3-yl)- |
| | amine |
| 241 | (3-chloro-phenyl)-(5-nitro- |
| | benzo[d]isothiazol-3-yl)-amine |
| 242 | (3-chloro-phenyl)-(2-thiophen-2- |
| | yl-oxazolo[5,4-d]pyrimidin-7-yl)- |
| | amine |
| 243 | (5-chloro-l-methyl-1H- |
| | thieno[2,3-c]pyrazol-3-yl)-(3- |
| | chloro-phenyl)-amine |
| 244 | (3-chloro-phenyl)-[5-methoxy-l- |
| | (2-pyrrolidin-l-yl-ethyl)-1H- |
| | indazol-3-yl]-amine (3-chloro- |
| | phenyl)-(5-methoxy-l- |
| | methoxymethyl-l H-indazol-3- |
| | yl)-amine |
| 245 | (3-chloro-phenyl)-[5-methoxy-l- |
| | (2-morpholin-4-yl-ethyl)-l H- |
| | indazol-3-yl]-amine |
| 246 | (3-chloro-phenyl)-(6,8-diiodo- |
| | quinazolin-4-yl)-amine |
| 247 | (3-chloro-phenyl)-(6-fluoro- |
| | chroman-4-yl)-amine |
| 248 | l-[3-(3-chloro-phenylamino)-5- |
| | methoxy-indazol-l-yl]-2- |
| | morpholin-4-yl-ethanone 3-[3-(3- |
| | chloro-phenylamino)-indazol-1- |
| | yl]-N,N-dimethyl-benzamide |
| 249 | (3-chloro-phenyl)-(5-methoxy-l- |
| | pyridin-4-ylmethyl-l H-indazol-3- |
| | yl)-amine |
| 250 | 4-chloro-2-(7H-pyrrolo[2,3- |
| | d]pyrimidin-4-ylamino)-phenol |
| 25 | (3-chloro-phenyl)-chroman-4-yl- |
| | amine |
| 252 | (5-chloro-2-methoxy-phenyl)- |
| | (7H-pyrrolo[2,3-d]pyrimidin-4- |
| | yl)-amine |
| 253 | (6-chloro-pyridin-2-yl)-(7H- |
| | pyrrolo[2,3-d]pyrimidin-4-yl)- |
| | amine |
| 254 | (3-chloro-2-fluoro-phenyl)-(7H- |
| | pyrrolo[2,3-d]pyrimidin-4-yl)- |
| | amine |
| 255 | [2-(2-bromo-phenyl)-ethyl]-(7H- |
| | pyrrolo[2,3-d]pyrimidin-4-yl)- |
| | amine |
| 256 | [2-(4-bromo-phenyl)-ethyl]-(7H- |
| | pyrrolo[2,3-d]pyrimidin-4-yl)- |
| | amine |
| 257 | (3-chloro-phenyl)-(5-methoxy-1- |
| | pyridin-3-ylmethyl-1H-indazol-3- |
| | yl)-amine |
| 258 | (3-chloro-phenyl)-(5-methoxy-1- |
| | pyridin-2-ylmethyl-1H-indazol-3- |
| | yl)-amine |
| 259 | (1-Benzyl-5-methoxy-1H-indazol- |
| | 3-yl)-(3-chloro-phenyl)-amine |
| 260 | (3-chloro-5-fluoro-phenyl)-(7H- |
| | pyrrolo[2,3-d]pyrimidin-4-yl)- |
| | amine |
| 261 | (3-chloro-phenyl)-quinolin-5-yl- |
| | amine |
| 262 | (3-chloro-phenyl)-(5-methoxy-l- |
| | thiazol-4-ylmethyl-l H-indazol-3- |
| | yl)-amine |
| 263 | (3-chloro-phenyl)-(5-methoxy-1- |
| | pyridin-3-yl-1H-indazol-3-yl)- |
| | amine |
| 264 | (3-chloro-4-fluoro-phenyl)-(7H- |
| | pyrrolo[2,3-d]pyrimidin-4-yl)- |
| | amine |
| 265 | N⁴-(3-chloro-phenyl)-7H- |
| | pyrrolo[2,3-d]pyrimidine-2,4- |
| | diamine |
| 266 | 2-[3-(3-chloro-phenylamino)-5- |
| | methoxy-indazol-l-yl]-N-methyl- |
| | acetamide |
| 267 | (5-bromo-1H-pyrazolo[3,4- |
| | b]pyridin-3-yl)-(3-chloro- |
| | phenyl)-amine |
| 268 | (3-chloro-phenyl)-[1-(1- |
| | isobutyl-1H-pyrazol-4-yl)-5- |
| | methoxy-1H-indazol-3-yl]-amine |
| 269 | (3-chloro-phenyl)-[5-methoxy-l- |
| | (tetrahydro-pyran-2-ylmethyl)- |
| | 1H-indazol-3-yl]-amine |
| 270 | (3-chloro-phenyl)-[5-methoxy-l- |
| | (2-pyrrol-l-yl-ethyl)-1H-indazol- |
| | 3-yl]-amine |
| 271 | (7H-pyrrolo[2,3-d]pyrimidin-4- |
| | ylamino)-acetic acid ethyl ester |
| 272 | (3-chloro-phenyl)-[5-methoxy-l- |
| | (l-phenyl-ethyl)-1H-indazol-3- |
| | yl]-amine |
| 273 | 3-[3-(3-chloro-phenylamino)-5- |
| | methoxy-indazol-l-yl]-N,N- |
| | dimethyl-benzamide |
| 274 | (3-chloro-phenyl)-[5-methoxy-l- |
| | (tetrahydro-pyran-4-ylmethyl)- |
| | 1H-indazol-3-yl]-amine |
| 275 | (3-chloro-phenyl)-[5-methoxy-l- |
| | (tetrahydro-furan-2-ylmethyl)- |
| | 1H-indazol-3-yl]-amine |
| 276 | (3-chloro-phenyl)-[5-methoxy-l- |
| | (5-methyl-isoxazol-3-ylmethyl)- |
| | 1H-indazol-3-yl]-amine |
| 277 | 2-[3-(3-chloro-phenylamino)-5- |
| | methoxy-indazol-1-yl]-1-phenyl- |
| | ethanone |
| 278 | 2-[3-(3-chloro-phenylamino)-5- |
| | methoxy-indazol-l-yl]-l-(2- |
| | methoxy-phenyl)-ethanone |
| 279 | 2-[3-(3-chloro-phenylamino)-5- |
| | methoxy-indazol-1-yl]-1-(3- |
| | methoxy-phenyl)-ethanone |
| 280 | 2-[3-(3-chloro-phenylamino)-5- |
| | methoxy-indazol-l-yl]-l-phenyl- |
| | ethanol |
| 28 | 3-[3-(3-chloro-phenylamino)-5- |
| | methoxy-indazol-1-yl]- |
| | benzonitrile |
| 282 | (3-chloro-phenyl)-[1-(2- |
| | diethylamino-ethyl)-5-methoxy- |
| | 1H-indazol-3-yl]-amine |
| 283 | (3-chloro-phenyl)-[5-methoxy-1- |
| | (2-methyl-thiazol-4-ylmethyl)- |
| | 1H-indazol-3-yl]-amine |
| 284 | 2-{2-[3-(3-chloro-phenylamino)- |
| | 5-methoxy-indazol-1-yl]-ethyl}- |
| | isoindole-1,3-dione |
| 285 | 1-{3-[3-(3-chloro-phenylamino)- |
| | 5-methoxy-indazol-1-yl]- |
| | phenyl}-ethanone |
| 286 | [5-bromo-l-(4-methoxy-benzyl)- |
| | 1H-pyrazolo[3,4-b]pyridin-3-yl]- |
| | (3-chloro-phenyl)-amine |
| 287 | 1-{3-[3-(3-chloro-phenylamino)- |
| | 5-methoxy-indazol-1-yl]- |
| | phenyl}-ethanol |
| 288 | (l-Allyl-5-methoxy-1H-indazol-3- |
| | yl)-(3-chloro-phenyl)-amine |
| 289 | (3-chloro-phenyl)-[l-(2-fluoro- |
| | benzyl)-5-methoxy-1H-indazol-3- |
| | yl]-amine |
| 290 | (3-chloro-phenyl)-[l-(3-fluoro- |
| | benzyl)-5-methoxy-1H-indazol-3- |
| | yl]-amine |
| 291 | (3-chloro-phenyl)-[l-(4-fluoro- |
| | benzyl)-5-methoxy-1H-indazol-3- |
| | yl]-amine |
| 292 | 2-[3-(3-chloro-phenylamino)-5- |
| | methoxy-indazol-1-yl]-1-phenyl- |
| | propan-1-one |
| 293 | 3-[3-(3-chloro-phenylamino)-5- |
| | methoxy-indazol-l-yl]-benzoic |
| | acid methyl ester |
| 294 | (3-chloro-phenyl)-[l-(3,5- |
| | difluoro-benzyl)-5-methoxy-1H- |
| | indazol-3-yl]-amine |
| 295 | (3-chloro-phenyl)-[5-methoxy-l- |
| | (2-trifluoromethoxy-benzyl)-1H- |
| | indazol-3-yl]-amine |
| 296 | 3-[3-(3-chloro-phenylamino)-5- |
| | methoxy-indazol-1-yl]-benzoic |
| | acid |
| 297 | (l-sec-butyl-5-methoxy-1H- |
| | indazol-3-yl)-(3-chloro-phenyl)- |
| | amine |
| 298 | (3-chloro-phenyl)-{1-[2-(4- |
| | fluoro-phenoxy)-ethyl]-5- |
| | methoxy-1H-indazol-3-yl}-amine |
| 299 | (3-chloro-phenyl)-[1-(3- |
| | methanesulfonyl-phenyl)-5- |
| | methoxy-1H-indazol-3-yl]-amine |
| 300 | 3-[3-(3-chloro-phenylamino)-5- |
| | methoxy-indazol-1-yl]- |
| | benzaldehyde |
| 301 | (3-chloro-phenyl)-{5-methoxy-1- |
| | [3-(pyrrolidine-1-sulfonyl)- |
| | phenyl]-l H-indazol-3-yl}-amine |
| 302 | (3-chloro-phenyl)-{1-[2-(1H- |
| | indol-3-yl)-ethyl]-5-methoxy-1H- |
| | indazol-3-yl}-amine (l- |
| | benzo[l,2,5]thiadiazol-4- |
| | ylmethyl-5-methoxy-1H-indazol- |
| | 3-yl)-(3-chloro-phenyl)-amine |
| 303 | 3-[3-(3-chloro-phenylamino)-5- |
| | methoxy-indazol-l-yl]-2,2- |
| | dimethyl-propan-l-ol |
| 304 | (l-benzenesulfonyl-5-methoxy- |
| | 1H-indazol-3-yl)-(3-chloro-4- |
| | fluoro-phenyl)-amine |
| 305 | (3-chloro-4-fluoro-phenyl)-(5- |
| | methoxy-1H-indazol-3-yl)-amine |
| 306 | 3-[3-(3-chloro-phenylamino)-5- |
| | methoxy-indazol-1-yl]-N-methyl- |
| | benzamide |
| 307 | (3-chloro-phenyl)-{5-methoxy-1- |
| | [3-(tetrahydro-pyran-2-yloxy)- |
| | propyl]-1H-indazol-3-yl}-amine |
| 308 | 3-[3-(3-chloro-phenylamino)-5- |
| | methoxy-indazol-1-yl]-N- |
| | isopropyl-benzamide |
| 309 | 3-[3-(3-chloro-phenylamino)-5- |
| | methoxy-indazol-l-yl]-propane- |
| | l,2-diol |
| 310 | 3-[3-(3-chloro-phenylamino)-5- |
| | methoxy-indazol-1-yl]-propan-1- |
| | ol |
| 311 | (3-chloro-phenyl)-[5-methoxy-l- |
| | (2-phenyl-thiazol-4-ylmethyl)- |
| | 1H-indazol-3-yl]-amine |
| 312 | (3-chloro-phenyl)-[5-methoxy-l- |
| | (2-thiophen-2-yl-thiazol-4- |
| | ylmethyl)-1H-indazol-3-yl]- |
| | amine |
| 313 | (3-chloro-4-fluoro-phenyl)-(6,8- |
| | difluoro-quinolin-4-yl)-amine |
| 314 | (3-chloro-4-fluoro-phenyl)-(6- |
| | methyl-thieno[2,3-d]pyrimidin-4- |
| | yl)-amine |
| 315 | (3-chloro-4-fluoro-phenyl)- |
| | isoquinolin-l-yl-amine |
| 316 | (3-chloro-4-fluoro-phenyl)-(6- |
| | trifluoromethyl-quinolin-4-yl)- |
| | amine |
| 317 | (3-chloro-4-fluoro-phenyl)-(6- |
| | fluoro-quinolin-4-yl)-amine |
| 318 | (3-chloro-phenyl)-{1-[3- |
| | (isopropylamino-methyl)- |
| | phenyl]-5-methoxy-1H-indazol- |
| | 3-yl}-amine |
| 319 | (3-chloro-phenyl)-{5-methoxy-1- |
| | [(S)-1-(tetrahydro-pyran-2- |
| | yl)methyl]-1H-indazol-3-yl}- |
| | amine |
| 320 | (3-chloro-phenyl)-{5-methoxy-1- |
| | [(R)-1-(tetrahydro-pyran-2- |
| | yl)methyl]-1H-indazol-3-yl}- |
| | amine |
| 321 | 3-[3-(3-chloro-phenylamino)-5- |
| | methoxy-indazol-l-yl]-N-(2,4- |
| | dimethoxy-benzyl)-benzamide |
| 322 | 3-[3-(3-chloro-phenylamino)-5- |
| | methoxy-indazol-1-yl]-N,N- |
| | dimethyl-benzenesulfonamide |
| 323 | (3-chloro-phenyl)-(2-chloro- |
| | quinazolin-4-yl)-amine |
| 324 | (3-chloro-phenyl)-[1-(2-[1,3] |
| | dioxolan-2-yl-ethyl)-5-methoxy- |
| | 1H-indazol-3-yl]-amine |
| 325 | 4-(3-chloro-phenylamino)- |
| | naphthalene-1-carbonitrile |
| 326 | N⁴-(3-chloro-phenyl)-N²-pentyl- |
| | quinazoline-2,4-diamine |
| 327 | (3-chloro-phenyl)-(2-morpholin- |
| | 4-yl-quinazolin-4-yl)-amine |
| 328 | 4-(3-chloro-phenylamino)- |
| | quinazoline-2-carboxylic acid |
| | ethyl ester |
| 329 | N²-benzyl-N⁴-(3-chloro-phenyl)- |
| | quinazoline-2,4-diamine |
| 330 | N⁴-(3-chloro-phenyl)-N²-(2- |
| | methoxy-ethyl)-N²-methyl- |
| | quinazoline-2,4-diamine |
| 331 | 4-[3-(3-chloro-phenylamino)-5- |
| | methoxy-indazol-l-yl]-butan-2- |
| | one |
| 332 | (3-chloro-2-fluoro-phenyl)-(6- |
| | trifluoromethyl-quinolin-4-yl)- |
| | amine |
| 333 | (3-chloro-5-fluoro-phenyl)-(6- |
| | trifluoromethyl-quinolin-4-yl)- |
| | amine |
| 334 | (5-chloro-2-fluoro-phenyl)-(6- |
| | trifluoromethyl-quinolin-4-yl)- |
| | amine |
| 335 | (3-chloro-phenyl)-[5-methoxy-l- |
| | (2-methoxy-ethyl)-1H-indazol-3- |
| | yl]-amine |
| 336 | 2-[3-(3-chloro-phenylamino)-5- |
| | methoxy-indazol-1-yl]-N,N- |
| | dimethyl-acetamide |
| 337 | 3-[3-(3-chloro-phenylamino)-5- |
| | methoxy-indazol-1-yl]-butan-2- |
| | one |
| 338 | 2-[3-(3-chloro-phenylamino)-5- |
| | methoxy-indazol-1-yl]- |
| | propionitrile |
| 339 | (3-chloro-phenyl)-[l-(3- |
| | ethanesulfonyl-phenyl)-5- |
| | methoxy-1H-indazol-3-yl]-amine |
| | (3-chloro-2-fluoro-phenyl)- |
| | isoquinolin-l-yl-amine |
| 340 | (3-chloro-5-fluoro-phenyl)- |
| | isoquinolin-l-yl-amine |
| 341 | [l-(2-amino-ethyl)-5-methoxy- |
| | 1H-indazol-3-yl]-(3-chloro- |
| | phenyl)-amine |
| 342 | 5-methoxy-1-(4-methoxy- |
| | benzyl)-1H-pyrazolo [3,4- |
| | b]pyridin-3-ylamine |
| 343 | 4-[3-(3-chloro-phenylamino)-5- |
| | methoxy-indazol-l-yl]-butan-2-ol |
| 344 | 3-[3-(3-chloro-phenylamino)-5- |
| | methoxy-indazol-1-yl]-butan-2-ol |
| 345 | (5-chloro-2-fluoro-phenyl)- |
| | isoquinolin-l-yl-amine |
| 346 | N⁴-(3-chloro-phenyl)-N²- |
| | phenethyl-quinazoline-2,4- |
| | diamine |
| 347 | N²-benzyl-N⁴-(3-chloro-phenyl)- |
| | N²-methyl-quinazoline-2,4- |
| | diamine |
| 348 | N⁴-(3-chloro-phenyl)-N²-(2- |
| | methoxy-ethyl)-quinazoline-2,4- |
| | diamine |
| 349 | 4-(3-chloro-phenylamino)- |
| | quinazoline-2-carboxylic acid |
| 350 | (3-chloro-phenyl)-[l-(4- |
| | methanesulfonyl-phenyl)-5- |
| | methoxy-1H-indazol-3-yl]-amine |
| 351 | 3-[3-(3-chloro-phenylamino)-5- |
| | methoxy-indazol-1-yl]- |
| | benzenesulfonamide |
| 352 | (3-chloro-phenyl)-(5-methoxy- |
| | 1H-pyrazolo[3,4-b]pyridin-3-yl)- |
| | amine |
| 353 | N⁴-(3-chloro-phenyl)-N²-phenyl- |
| | quinazoline-2,4-diamine or |
| 354 | 2-pyrazin-2-yl-thiazole-4- |
| | carboxylic acid (2-pyridin-2-yl- |
| | ethyl)-amide. |

Other illustrative, but non-limiting D5D inhibitors include, but are not limited to CP 24,879, (4-(3-methylbutoxy)-benzenamine, monohydrochloride)

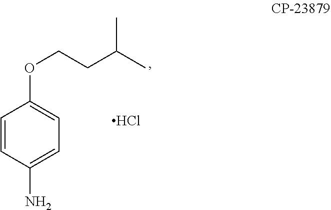

T3364366 (N-[2-[3,4-Dihydro-4-oxo-3-[4-(2,2,2-trifluoroethoxy)phenyl]thieno[3,4-d]pyrimidin-2-yl]thio]ethyl]acetamide):

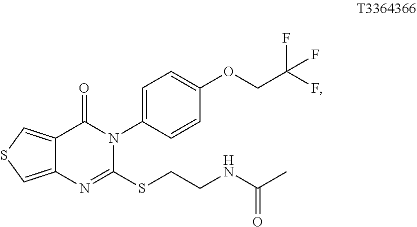 [SMILES: CC(=O)NCCSc1nc2cscc2c(=O)n1-c1ccc(OCC(F)(F)F)cc1]

and/or D5D-IN-326 (2-(2,2,3,3,3-Pentafluoropropoxy)-3-[4-(2,2,2-trifluoroethoxy) phenyl]-5,7-dihydro-3H-pyrrolo[2,3-d]pyrimidine-4,6-dione, CAS No.: 1236767-85-3) described by Takagahara et al. (2016) PLoS ONE, 11(11): e0166198 the structure of which is shown below:

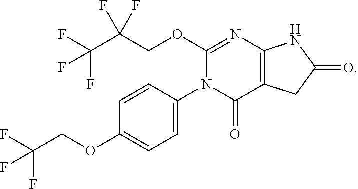 [SMILES: O=C1Cc2c(nc(OCC(F)(F)C(F)(F)F)n(-c3ccc(OCC(F)(F)F)cc3)c2=O)N1]

The foregoing D5D inhibitors are illustrative and non-limiting. Using the teachings provided herein, numerous other D5D inhibitors for use in the methods described herein will be recognized by one of skill in the art.

### Administration

In certain aspects, a prophylactically effective and/or a therapeutically effective amount of one or more of the active agents described herein (e.g., DGLA, and/or GLA, and/or D5D inhibitor) alone or in combination with one or more additional senolytic agents may be administered to a subject. Administration is performed using standard effective techniques, including peripherally (e.g., not by administration into the central nervous system) or locally to the central nervous system. Peripheral administration includes but is not limited to oral, inhalation, intravenous, intraperitoneal, intra-articular, subcutaneous, pulmonary, transdermal, intramuscular, intranasal, buccal, sublingual, or suppository administration. Local administration, including directly into the central nervous system (CNS), includes but is not limited to via a lumbar, intraventricular or intraparenchymal catheter or using a surgically implanted controlled release formulation. The route of administration may be dictated by the disease or condition to be treated. For example, if the disease or condition is COPD or IPF, the composition may be administered via inhalation. Alternatively, if the disease or condition is osteoarthritis, the composition may be administered via intra-articular administration. It is within the skill of one in the art, to determine the route of administration based on the disease or condition to be treated. In a specific embodiment, a composition of the invention is administered orally.

Pharmaceutical compositions comprising one or more of the active agents described herein (e.g., DGLA, and/or GLA, and/or D5D inhibitor) alone or in combination with one or more additional senolytic agents for effective administration are deliberately designed to be appropriate for the selected mode of administration, and pharmaceutically acceptable carriers such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners.

For therapeutic applications, a therapeutically effective amount of DGLA alone or in combination with one or more additional senolytic agents is administered to a subject. A “therapeutically effective amount” is an amount of the therapeutic composition sufficient to produce a measurable response (e.g., cell death of senescent cells, an anti-aging response, a delay in the onset of or progression of or improvement in symptoms associated with a degenerative disease, a delay in the onset of or progression of or an improvement in symptoms associated with a function-decreasing disorder, or a delay in the onset of, or progression of, or improvement in symptoms associated with a DNA damaging therapy). Actual dosage levels of active ingredients in a therapeutic composition of the invention can be varied so as to administer an amount of the active compound(s) that is effective to achieve the desired therapeutic response for a particular subject. The selected dosage level will depend upon a variety of factors, including the activity of the therapeutic composition, formulation, the route of administration, combination with other drugs or treatments, age, the age-related disease or condition, the degenerative disease, the function-decreasing disorder, the symptoms, and the physical condition and prior medical history of the subject being treated. In some embodiments, a minimal dose is administered, and the dose is escalated in the absence of dose-limiting toxicity. Determination and adjustment of a therapeutically effective dose, as well as evaluation of when and how to make such adjustments, are known to those of ordinary skill in the art of medicine.

In various embodiments, the frequency of dosing may be daily or once, twice, three times or more per week or per month, as needed for prophylactic effect or as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments.

Dosages of DGLA and/or GLA, and/or D5D inhibitor alone or in combination with one or more additional senolytic agents can vary between wide limits, depending upon the disease or disorder to be treated, the age and condition of the subject to be treated. In an embodiment where a composition comprising DGLA and/or GLA, and/or D5D inhibitor alone or in combination with one or more additional senolytic agents is contacted with a sample, the concentration of the DGLA and/or GLA, and/or D5D inhibitor may be from about 1 μM to about 1000 μM. In certain embodiments, the concentration of DGLA and/or GLA, and/or D5D inhibitor may be from about 5 μM to about 25 μM. For example, the concentration of DGLA and/or GLA, and/or D5D inhibitor may be about 1, about 2.5 about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, or about 40 μM. Additionally, the concentration of the DGLA and/or GLA, and/or D5D inhibitor may be greater than 40 μM. For example, the concentration of DGLA and/or GLA, and/or D5D inhibitor may be about 40, about 45, about 50, about 55, about 60, about 65, about 70, about 75, about 80, about 85, about 90, about 95 or about 100 μM.

In certain embodiments, the composition comprising a DGLA and/or GLA, and/or D5D inhibitor may be from about 0.1 mg/kg to about 500 mg/kg or higher, for example up to 2000 mg/kg of the active agent(s). For example, the dose of a DGLA and/or GLA, and/or D5D inhibitor may be about 0.1 mg/kg, about 0.5 mg/kg, about 1 mg/kg, about 5 mg/kg, about 10 mg/kg, about 15 mg/kg, about 20 mg/kg, or about 25 mg/kg. Alternatively, the dose of the DGLA may be about 25 mg/kg, about 50 mg/kg, about 75 mg/kg, about 100 mg/kg, about 125 mg/kg, about 150 mg/kg, about 175 mg/kg, about 200 mg/kg, about 225 mg/kg, or about 250 mg/kg. Additionally, in certain embodiments, the dose of DGLA and/or GLA, and/or D5D inhibitor may be about 300 mg/kg, about 325 mg/kg, about 350 mg/kg, about 375 mg/kg, about 400 mg/kg, about 425 mg/kg, about 450 mg/kg, about 475 mg/kg, about 500 mg/kg, about 1000 mg/kg or about 2000 mg/kg.

Typical dosage levels can be determined and optimized using standard clinical techniques and will be dependent on the mode of administration.

### Subject

A subject may be a rodent, a human, a livestock animal, a companion animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In still another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In a preferred embodiment, the subject is a human.

The human subject may be of any age. However, since senescent cells are normally associated with aging, a human subject may be an older human subject. In some embodiments, the human subject may be about 30, 35, 40, 45, 50, 55, 60, 65, 70, 75, 80, 85, 90, 95 years of age or older. In some preferred embodiments, the human subject is 30 years of age or older. In other preferred embodiments, the human subject is 40 years of age or older. In other preferred embodiments, the human subject is 45 years of age or older. In yet other preferred embodiments, the human subject is 50 years of age or older. In still other preferred embodiments, the human subject is 55 years of age or older. In other preferred embodiments, the human subject is 60 years of age or older. In yet other preferred embodiments, the human subject is 65 years of age or older. In still other preferred embodiments, the human subject is 70 years of age or older. In other preferred embodiments, the human subject is 75 years of age or older. In still other preferred embodiments, the human subject is 80 years of age or older. In yet other preferred embodiments, the human subject is 85 years of age or older. In still other preferred embodiments, the human subject is 90 years of age or older.

Additionally, a subject in need thereof may be a subject suffering from an age-related disease or condition as described above.

### Pharmaceutical Formulations.

In various embodiments, pharmaceutical formulations comprising DGLA and/or GLA, and/or D5D inhibitor are provided. In certain embodiments, the pharmaceutical formulation comprises DGLA and/or GLA, and/or D5D inhibitor and at least one “pharmaceutically acceptable carrier”.

In various embodiments illustrative, but non-limiting embodiments, the pharmaceutically acceptable carrier may comprise a diluent, a binder, a filler, a buffering agent, a pH modifying agent, a disintegrant, a dispersant, a preservative, a lubricant, taste-masking agent, a flavoring agent, and/or a coloring agent. The amount and types of carriers utilized to form pharmaceutical compositions may be selected according to known principles of pharmaceutical science.

In one illustrative, but non-limiting embodiment, the carrier may comprise a diluent. In various embodiments, the diluent may be compressible (i.e., plastically deformable) or abrasively brittle. Non-limiting examples of suitable compressible diluents include microcrystalline cellulose (MCC), cellulose derivatives, cellulose powder, cellulose esters (e.g., acetate and butyrate mixed esters), ethyl cellulose, methyl cellulose, hydroxypropyl cellulose, hydroxypropyl methylcellulose, sodium carboxymethylcellulose, corn starch, phosphated corn starch, pregelatinized corn starch, rice starch, potato starch, tapioca starch, starch-lactose, starch-calcium carbonate, sodium starch glycolate, glucose, fructose, lactose, lactose monohydrate, sucrose, xylose, lactitol, mannitol, malitol, sorbitol, xylitol, maltodextrin, and trehalose. Non-limiting examples of suitable abrasively brittle diluents include dibasic calcium phosphate (anhydrous or dihydrate), calcium phosphate tribasic, calcium carbonate, and magnesium carbonate.

In certain embodiments, the carrier may comprise a binder. Suitable binders include, but are not limited to, starches, pregelatinized starches, gelatin, polyvinylpyrrolidone, cellulose, methylcellulose, sodium carboxymethylcellulose, ethylcellulose, polyacrylamides, polyvinyloxoazolidone, polyvinylalcohols, C12-C18 fatty acid alcohol, polyethylene glycol, polyols, saccharides, oligosaccharides, polypeptides, oligopeptides, and combinations thereof.

In certain embodiments, the carrier may comprise a filler. Suitable fillers include, but are not limited to, carbohydrates, inorganic compounds, and polyvinylpyrrolidone. By way of non-limiting example, the filler may be calcium sulfate, both di- and tri-basic, starch, calcium carbonate, magnesium carbonate, microcrystalline cellulose, dibasic calcium phosphate, magnesium carbonate, magnesium oxide, calcium silicate, talc, modified starches, lactose, sucrose, mannitol, or sorbitol.

In certain embodiments, the carrier may comprise a buffering agent. Representative examples of suitable buffering agents include, but are not limited to, phosphates, carbonates, citrates, tris buffers, and buffered saline salts (e.g., Tris buffered saline or phosphate buffered saline, and the like).

In various embodiments, the carrier may comprise a pH modifier. By way of non-limiting example, in certain embodiments, the pH modifying agent may comprise sodium carbonate, sodium bicarbonate, sodium citrate, citric acid, or phosphoric acid.

In certain embodiments, the carrier may comprise a disintegrant. The disintegrant may be non-effervescent or effervescent. Suitable examples of non-effervescent disintegrants include, but are not limited to, starches such as corn starch, potato starch, pregelatinized and modified starches thereof, sweeteners, clays, such as bentonite, micro-crystalline cellulose, alginates, sodium starch glycolate, gums such as agar, guar, locust bean, karaya, pecitin, and tragacanth. Non-limiting examples of suitable effervescent disintegrants include sodium bicarbonate in combination with citric acid and sodium bicarbonate in combination with tartaric acid.

In certain embodiments, the carrier may comprise a dispersant or dispersing enhancing agent. Suitable dispersants may include, but are not limited to, starch, alginic acid, polyvinylpyrrolidones, guar gum, kaolin, bentonite, purified wood cellulose, sodium starch glycolate, isoamorphous silicate, and microcrystalline cellulose.

In certain embodiments, the carrier may comprise a preservative. Non-limiting examples of suitable preservatives include antioxidants, such as BHA, BHT, vitamin A, vitamin C, vitamin E, or retinyl palmitate, citric acid, sodium citrate; chelators such as EDTA or EGTA; antimicrobials, such as parabens, chlorobutanol, or phenol; and the like.

In certain embodiments, the carrier may comprise be a lubricant. Non-limiting examples of suitable lubricants include minerals such as talc or silica and/or fats such as vegetable stearin, magnesium stearate or stearic acid.

In certain embodiments, the carrier may comprise a taste-masking agent. Taste-masking materials include cellulose ethers, polyethylene glycols, polyvinyl alcohol, polyvinyl alcohol and polyethylene glycol copolymers, monoglycerides or triglycerides, acrylic polymers, mixtures of acrylic polymers with cellulose ethers, cellulose acetate phthalate, and combinations thereof.

In certain embodiments, the carrier may comprise a flavoring agent. In certain embodiments, flavoring agents may be chosen from synthetic flavor oils and flavoring aromatics and/or natural oils, extracts from plants, leaves, flowers, fruits, and combinations thereof.

In certain embodiments, the carrier may comprise a coloring agent. Suitable color additives include, but are not limited to, food, drug and cosmetic colors (FD&C), drug and cosmetic colors (D&C), or external drug and cosmetic colors (Ext. D&C).

In certain embodiments, the weight fraction of the carrier or combination of carriers in the composition may be about 99% or less, about 97% or less, about 95% or less, about 90% or less, about 85% or less, about 80% or less, about 75% or less, about 70% or less, about 65% or less, about 60% or less, about 55% or less, about 50% or less, about 45% or less, about 40% or less, about 35% or less, about 30% or less, about 25% or less, about 20% or less, about 15% or less, about 10% or less, about 5% or less, about 2%, or about 1% or less of the total weight of the composition.

In certain embodiments, composition can be formulated into various dosage forms and administered by a number of different means that will deliver a therapeutically effective or prophylactically effective amount of the active ingredient(s) (DGLA and/or GLA, and/or D5D inhibitor). Such compositions can be administered orally, parenterally, or topically in dosage unit formulations containing conventional nontoxic pharmaceutically acceptable carriers, adjuvants, and vehicles as desired. Topical administration may also involve the use of transdermal administration such as transdermal patches or iontophoresis devices. The term parenteral as used herein includes subcutaneous, intravenous, intramuscular, or intrasternal injection, or infusion techniques. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980). In one embodiment, the composition may comprise a food supplement or the composition may comprise a cosmetic.

Solid dosage forms for oral administration include capsules, tablets, caplets, pills, powders, pellets, and granules. In such solid dosage forms, the active ingredient is ordinarily combined with one or more pharmaceutically acceptable carriers, examples of which are detailed above. Oral preparations may also be administered as aqueous suspensions, elixirs, or syrups. For these, the active ingredient may be combined with various sweetening or flavoring agents, coloring agents, and, if so desired, emulsifying and/or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use.

In certain embodiments, extemporaneous injection solutions and suspensions may be prepared from sterile powders, granules and tablets.

For topical (e.g., transdermal or transmucosal) administration, penetrants appropriate to the barrier to be permeated are generally included in the preparation. Pharmaceutical compositions adapted for topical administration may be formulated as ointments, creams, suspensions, lotions, powders, solutions, pastes, gels, sprays, aerosols or oils. In some embodiments, the pharmaceutical composition is applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base. Pharmaceutical compositions adapted for topical administration to the eye include eye drops wherein the active ingredient is dissolved or suspended in a suitable carrier, especially an aqueous solvent. Pharmaceutical compositions adapted for topical administration in the mouth include lozenges, pastilles and mouth washes. Transmucosal administration may be accomplished through the use of nasal sprays, aerosol sprays, tablets, or suppositories, and transdermal administration may be via ointments, salves, gels, patches, or creams as generally known in the art.

In certain embodiments, a composition comprising DGLA and/or GLA, and/or D5D inhibitor, is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering DGLA and/or GLA, and/or D5D inhibitor. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one illustrative embodiment, a liposome delivery vehicle may be utilized. Liposomes, depending upon the embodiment, are suitable for delivery of DGLA and/or GLA alone or in combination with a D5D inhibitor and/or one or more additional senolytic agents described herein in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome and lipid bilayer to cells. DGLA and GLA are hydrophobic and readily incorporated in the lipid bilayer. In this manner, DGLA may be selectively delivered to a cell by incorporation into the lipid bilayer of a liposome that fuses with the targeted cell's membrane. It will be noted that in certain embodiments, a D5D inhibitor can be contained within the liposome. In certain embodiments one or more additional senolytic agents can be contained within the liposome, while DGLA is present in the lipid bilayer.

Liposomes comprising DGLA and/or GLA in the lipid bilayer optionally containing a D5D inhibitor and/or one or more additional senolytic agents may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar liposomes.

In another embodiment, DGLA and/or GLA, and/or D5D inhibitor alone or in combination with one or more senolytic agent(s) may be delivered to a cell as a microemulsion. Microemulsions are generally clear, thermodynamically stable solutions comprising an aqueous solution, a surfactant, and “oil.” The “oil” in this case DGLA and/or GLA, provides the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids and, in various embodiments the lipophilic agents described herein (e.g., GLA, DGLA). Any of the phospholipids detailed above for liposomes are suitable for embodiments directed to microemulsions. It will also be recognized that where DGLA and/or GLA are to be administered in conjunbation with another agent, e.g. another senolytic agent and/or a D5D inhibitor, the DGLA alone or in combination with one or more senolytic agent(s) may be encapsulated in the microemulsion by any method generally known in the art.

The foregoing methods, compositions, and formulations are illustrative and not limiting. Using the teaching provided herein numerous other methods, compositions, and formulations will be available to one of skill in the art.

Some animal models which are related to senescence-associated diseases or disorders are already well known in the art. For example, a mouse model related to Osteoarthritis which is one of the senescence-associated diseases is disclosed (Jeon O H et al. (2017) Nat Med 23(6): 775-781). Other mouse models related to senescence-associated diseases or disorders are also well known in the art, for example, progeroid model mice (Baker et al. (2011) Nature, 479(7372): 232-236; Zhang et al. (2017) Redox Biol. 11: 30-37), aged model mice (Baker et al. (2016) Nature, 530(7589): 184-189; Chang et al. (2016) Nat. Med. 22(1): 78-83; Xu et al. (2018) Nat. Med. 24(8): 1246-1256), irradiation-induced side-effect model mice (Chang et al. (2016) Nat. Med. 22(1): 78-83), chemotherapy-induced side effect model mice (Demaria et al. (2017) Cancer Discov, 7(2): 165-176; Baar et al. (2017) Cell, 169(1): 132-147.e16), idiopathic pulmonary fibrosis model mice (Schafer et al. (2017) Nat. Commun. 8: 14532; Wiley et al. (2019) JCI Insight, 4(24): 130056), experimental ocular hypertension model mice (Rocha et al. (2019) Aging Cell, 19(2): e13089), Parkinson's disease model mice (Chinta et al. (2018) Cell Rep. 22(4): 930-940), Alzheimer's disease model mice (Wei et al. (2016) Chin. Med. J. 129(15): 1835-44; Zhang et al. (2019) Nat. Neurosci. 22(5): 719-728), tau-dependent neurodegenerative model mice (Bussian et al. (2018) Nature, 562(7728): 578-582), diabetes and hepatic steatosis model mice (Ogrodnik et al. (2017) Nat. Commun. 8: 15691; Aguayo-Mazzucato et al. (2019) Cell Metab. 30(1): 129-142.e4) and atherosclerosis model mice (Childs et al. (2016) Science, 354(6311):472-477). These animal models can be used to investigate the effectiveness of DGLA alone or in combination with one or more additional senolytic agents.

### Kits.

In certain embodiments kits for the use of DGLA and/or GLA, and/or a D5D inhibitor alone or in combination with one or more additional senolytic agents are provided. Typically, such kit will comprise a container containing DGLA alone or in combination with one or more additional senolytic agents. In certain embodiments the DGLA alone or in combination with one or more additional senolytic agents can be provided in a unit dosage formulation (e.g., vial, tablet, caplet, patch, etc.) and/or may be optionally combined with one or more pharmaceutically acceptable excipients.

In addition, in certain embodiments, the kits optionally include labeling and/or instructional materials providing directions (i.e., protocols) for the use of DGLA and/or GLA, and/or a D5D inhibitor alone or in combination with one or more additional senolytic agents as described herein. Thus, for example, the kit may contain directions for the use of DGLA alone or in combination with one or more additional senolytic agents in the treatment of the treatment of cancer and/or the prevention of therapy induced senescent cells, and/or for delaying the onset or progression of age-related diseases.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit, the claimed invention.

### Example 1

### Identification of Dihomo-γ-Linolenic Acid (DGLA) as a Senolytic Agent

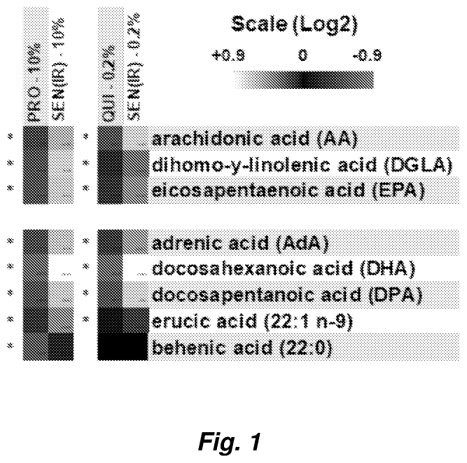

It has been demonstrated that senescent cells accumulate signaling lipids and their polyunsaturaged fatty acid (PUFA) precursors. These precursors include, but are not limited to Dihomo-γ-Linolenic Acid (DGLA) (see, e.g., FIG. 1).

DGLA was measured in either proliferating (PRO—10% FBS) or irradiation-induced senescent IMR-90 fibroblasts [SEN(IR)—10% FBS] 10 days after treatment, and relative abundances were measured by mass spectrometry and normalized to total protein (BCA assay). As shown in FIG. 14, panel A, DGLA shows significantly higher abundance in senescent cells. MiDAS (mitochondrial dysfunction induces a senescence) was induced by serial passage of IMR-90 fibroblasts in the presence of ethidium bromide to deplete mitochondrial DNA, followed by pyruvate depletion and culture in 0.2% FBS (MiDAS-0.2% FBS). As shown in FIG. 15, panel B, DGLA was significantly increased in senescent (IR induced) cells and significantly higher in the MiDAS cells.

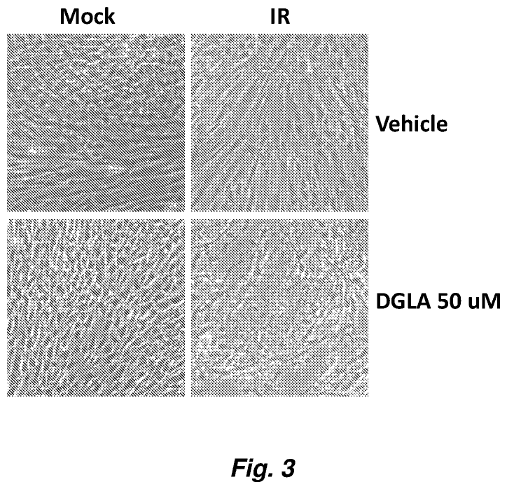

To evaluate the effect of DGLA on senescent cells, IMR-90 fibroblasts were either mock-irradiated (Mock) or irradiated with 10Gy of ionization radiation (IR) to induce senescence. Ten days later cells were cultured in the presence of either vehicle (media plus FBS carrier) or 50 micromolar DGLA for 2 days. Cells were then photographed using light microscopy. As shown in FIG. 3 DGLA was selectively toxic to senescent cells.

Toxic reactive carbon species such as 8-HOA are made as minority products of DGLA oxygenation by COX-2. In particular, DGLA is peroxidated on carbon 8 by COX-2 as a minority product of the enzyme activity. Beta-scission on either side of this residue results in formation of either a heptanoic acid radical, or an 8-hydroxy-octanoic acid (8-HOA) radical. These are toxic to the cell and induce apoptosis.

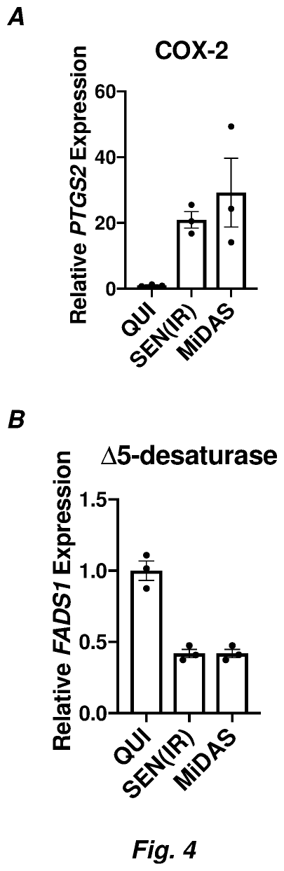

In view of this, one potential vulnerability of senescent cells is that they elevate prostaglandin synthase 2 expression (aka COX-2, gene name PTGS2) as illustrated in FIG. 4, panel A. This is coupled to a loss of 45-desaturase (aka D5D, gene name FADS1), an enzyme that desaturates PUFAs as illustrated in FIG. 4, panel B.

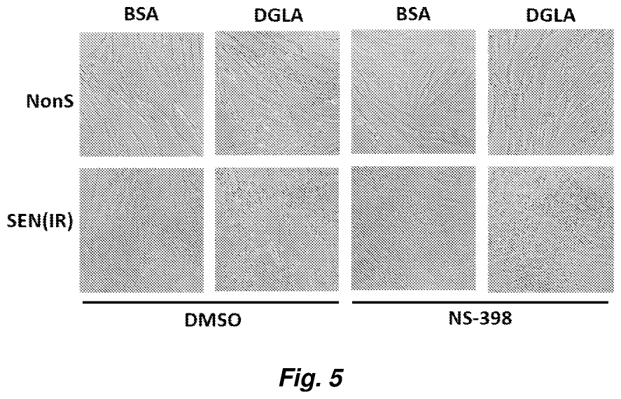

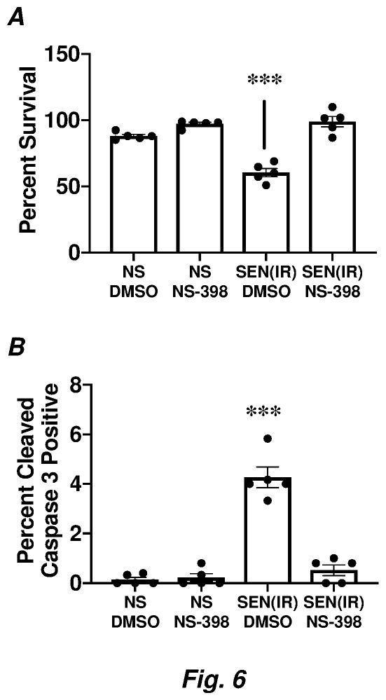

As shown in FIG. 5 and FIG. 6, panels A and B, we found that the endogenous lipid, DGLA, kills senescent cells in a COX-2-dependent manner. Since DGLA is converted to non-toxic arachidonic acid by D5D in most normal cells (Nakamura & Nara (2004) Annu. Rev. Nutr. 24: 345-376), the combination of gain of COX-2 and loss of D5D in senescent cells makes this an exploitable weakness. As an endogenous lipid that is only likely to be toxic to senescent cells, DGLA is a potentially superior option to most current senolytics. Its derivatives, the 1-series prostaglandins (PGX1's) are largely anti-inflammatory, and therefore may also have positive effects in the context of sterile inflammation associated with aging, so-called “inflammaging” (Franceschi & Campisi (2014) J. Gerontol. A Biol. Sci. Med. Sci. 69 (Suppl 1): S4-9).

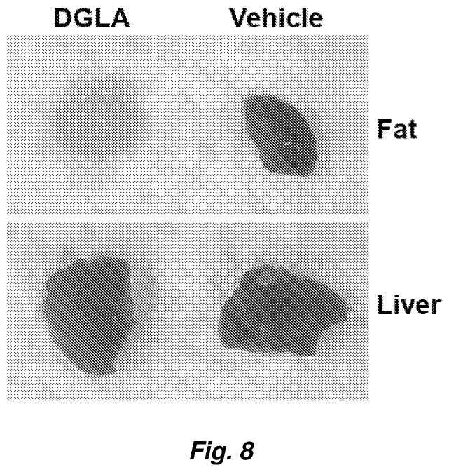

As shown in FIG. 8, less senescence-associated beta-glactodsidase was observed in tissues from DGLA-treated mice.

### REFERENCES

1. Coppe, J. P., et al., Senescence-associated secretory phenotypes reveal cell-nonautonomous functions of oncogenic RAS and the p53 tumor suppressor. PLoS Biol, 2008. 6(12): 2853-2868.
2. Wiley, C. D. and J. Campisi, From Ancient Pathways to Aging Cells-Connecting Metabolism and Cellular Senescence. Cell Metab, 2016. 23(6): 1013-1021.
3. Basisty, N., et al., A proteomic atlas of senescence-associated secretomes for aging biomarker development. PLoS Biol, 2020. 18: p. e3000599.
4. Kuilman, T., et al., The essence of senescence. Genes Dev, 2010. 24(22): p. 2463-2479.
5. Baker, D. J., et al., Naturally occurring p16(Ink4a)-positive cells shorten healthy lifespan. Nature, 2016. 530(7589): p. 184-189.
6. Farr, J. N., et al., Targeting cellular senescence prevents age-related bone loss in mice. Nat Med, 2017. 23(9): p. 1072-1079.
7. Thompson, P. J., et al., Targeted Elimination of Senescent Beta Cells Prevents Type 1 Diabetes. Cell Metab, 2019. 29(5): p. 1045-1060 e10.
8. Aguayo-Mazzucato, C., et al., Acceleration of beta Cell Aging Determines Diabetes and Senolysis Improves Disease Outcomes. Cell Metab, 2019. 30(1): p. 129-142 e4.
9. Palmer, A. K., et al., Targeting senescent cells alleviates obesity-induced metabolic dysfunction. Aging Cell, 2019: p. e12950.
10. Ozkurt, S., et al., Acute renal failure under dasatinib therapy. Ren Fail, 2010. 32(1): p. 147-149.
11. Gandhi, L., et al., Phase I study of Navitoclax (ABT-263), a novel Bcl-2 family inhibitor, in patients with small-cell lung cancer and other solid tumors. J Clin Oncol, 2011. 29(7): p. 909-916.
12. Nakamura, M. T. and T. Y. Nara, Structure, function, and dietary regulation of delta6, delta5, and delta9 desaturases. Annu Rev Nutr, 2004. 24: p. 345-376.
13. Franceschi, C. and J. Campisi, Chronic inflammation (inflammaging) and its potential contribution to age-associated diseases. J Gerontol A Biol Sci Med Sci, 2014. 69 Suppl 1: p. S4-9.
14. Kawashima, H., et al., Subchronic (13-week) oral toxicity study of dihomo-gamma-linolenic acid (DGLA) oil in rats. Food Chem Toxicol, 2009. 47(6): p. 1280-1286.
15. Yang, X., et al., Dihomo-gamma-linolenic acid inhibits growth of xenograft tumors in mice bearing human pancreatic cancer cells (BxPC-3) transfected with delta-5-desaturase shRNA. Redox Biol, 2019. 20: 236-246.

### Example 2

Evaluation of CP 24,879, (4-(3-methylbutoxy)-benzenamine, monohydrochloride) and T3364366 (N-[2-[[3,4-Dihydro-4-oxo-3-[4-(2,2,2-trifluoroethoxy)phenyl]thieno[3,4-d]pyrimidin-2-yl]thio]ethyl]acetamide.

CP 24,879, (4-(3-methylbutoxy)-benzenamine, monohydrochloride, CAS No: 10141-51-2) an inhibitor of both delta-5- and delta-6-desaturase originally described in Obukowicz et al. (1998) Biochem. Pharmacol. 55: 1045-1058, was obtained from Cayman Chemical. T3364366 (N-[2-[[3,4-Dihydro-4-oxo-3-[4-(2,2,2-trifluoroethoxy)phenyl|thieno[3,4-d]pyrimidin-2-yl]thio]ethyl]acetamide, CAS No: 1356354-09-0), a specific inhibitor of delta-5-desaturase (D5D, gene name: FADS1), described in Miyahisa et al. (2016) ACS Med. Chem. Lett. 7: 868-872, was obtained from R&D Systems Inc. In murine models, FADS1/D5D inhibitors elevate DGLA and lower AA, and are shown to attenuate pathologies associated with diet-induced obesity and atherosclerosis—conditions that are also improved by treatment with senolytics.

As described below, while CP 24,879 can prevent desaturation of DGLA by FADS1/D5D, it requires exogenous DGLA since it also inhibits FADS2/D6D, which is necessary for DGLA synthesis from shorter chain PUFAs. By comparison, T3364366 is a specific FADS1/D5D inhibitor, and is therefore capable of elevating intracellular DGLA so long as the cell has a source of short-chain PUFAs which are found in every bodily fluid as well as in the FBS provided to cultured cells.

### Methods and Results.

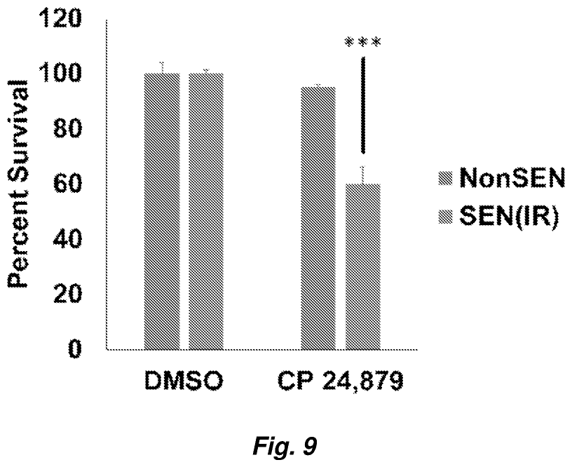

IMR-90 were induced to senesce via 10 Gy of ionizing radiation [SEN(IR)] and cultured for 7 days, followed by treatment with 11.75 μM CP 24,879 or vehicle (DMSO) in the presence of a sub-toxic concentration of DGLA (10 μM). Nonsenescent (NonSEN) cells served as a control. After 72 hours, cell numbers were counted and normalized to DMSO treatments. As shown in FIG. 9, inhibition of delta-5 desaturase selectively kills senescent cells treated with 10 μM DGLA.

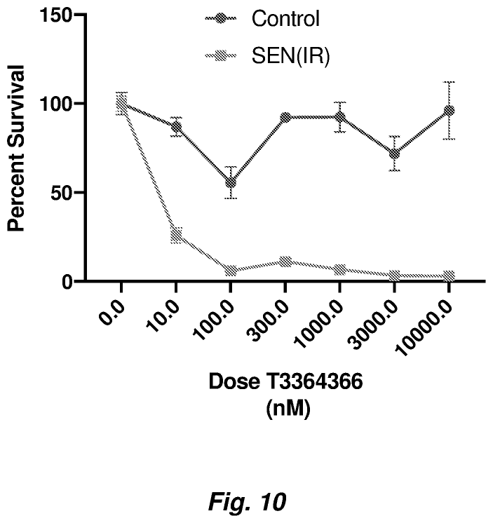

IMR-90 were induced to senesce via 10 Gy of ionizing radiation [SEN(IR)] and cultured for 7 days, followed by treatment with the indicated doses of T3364366 in the presence of 10% FBS (to provide PUFAs). Nonsenescent (NonSEN) cells served as a control. After 72 hours, cell numbers were counted and normalized to DMSO treatments. As shown in FIG. 10, T3364366, a D5D-specific inhibitor, selectively kills senescent cells.

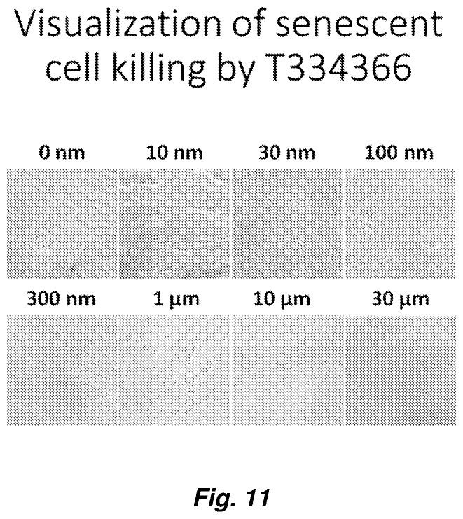

Cells from FIG. 10 were visualized by light microscopy, allowing visualization of the cytotoxic properties of T334366 at various doses. (see, e.g., FIG. 11).

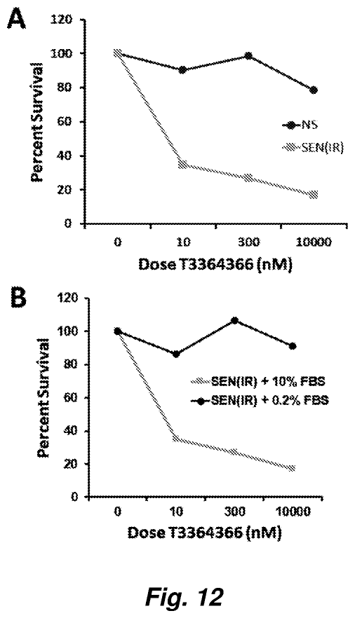

IMR-90 were induced to senesce via 10 Gy of ionizing radiation [SEN(IR)] and cultured for 7 days, followed by treatment with the indicated doses of T3364366 in the presence of 10% FBS (which provides PUFAs and growth factors) (FIG. 12, panels A and B—red line, squares) or 0.2% FBS (FIG. 12, panel B—purple line, circles) (which induces quiescence, but limits the ability of cells to synthesize DGLA). Nonsenescent (NonSEN) cells served as a control. After 72 hours, cell numbers were counted and normalized to the 0 dose treatment groups. As shown by a comparison of FIG. 12, panel A with panel B, T3364366 only kills in the presence of FBS.

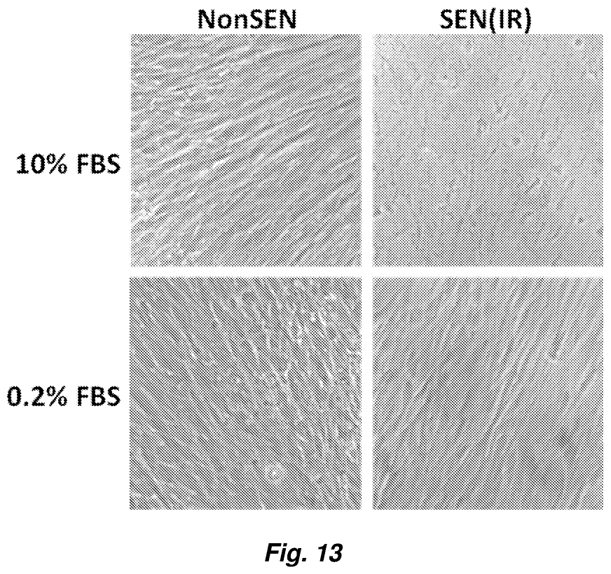

Cells from FIG. 12 were visualized by light microscopy, allowing visualization of the FBS-dependent killing of senescent cells by T334366 (see, e.g., FIG. 13).

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-87. (canceled)

88. A method of selectively killing one or more senescent cells in a subject in need thereof said method comprising:
administering to said subject an effective amount of one or more agents selected from the group consisting of dihomo-gamma-linolenic acid (DGLA), gamma-linolenic acid (GLA), and a delta-5-desaturase inhibitor (D5D inhibitor).

89. The method of claim 88, wherein said subject in need thereof is a subject that shows one or more features of aging in the subject, or wherein said subject in need thereof is a subject receiving DNA damaging or cytotoxic therapy, or wherein said subject in need thereof is a subject having a cancer.

90. The method of claim 88, wherein said subject has a cancer or pre-cancerous lesions.

91. The method of claim 90, wherein said subject has a cancer selected from the group consisting of leukemia, a secondary tumor, a solid tumor, acute leukemia, adrenal gland tumor, ameloblastoma, anaplastic carcinoma of the thyroid, angioma, apudoma, argentaffinoma, arrhenoblastoma, ascites tumor, astroblastoma, astrocytoma, ataxia-telangiectasia-associated tumors, basal cell carcinoma, bone cancer, brain tumor, brainstem glioma, breast cancer, Burkitt's lymphoma, cervical cancer, cholangioma, chondroblastoma, chondrosarcoma, chorioblastoma, choriocarcinoma, colon cancer, craniopharyngioma, cystocarcinoma, cystofbroma, cystoma, ductal carcinoma, ductal papilloma, dysgerminoma, encephaloma, endometrial carcinoma, endothelioma, ependymoma, erythroleukemia, Ewing's sarcoma, extra nodal lymphoma, fibro adenoma, fibro sarcoma, follicular cancer of the thyroid, ganglioglioma, gastrinoma cell, glioblastoma multiform, glioma, gonadoblastoma, haemangioblastoma, haemangioendothelioblastoma, haemangioendothelioma, haemangiopericytoma, haematolymphangioma, haemocytoblastoma, haemocytoma, hairy cell leukemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angioforoma, Kaposi sarcoma, kidney tumor, large cell lymphoma, leukemia, lipoma, liver cancer, liver metastases, Lucke carcinoma, lung cancer, lymphadenoma, lymphangioma, lymphocytic leukemia, lymphocytic lymphoma, lymphoedema, lymphoeytoma, lymphoma, malignant mesothelioma, malignant teratoma, mastocytoma, medulloblastome, melanoma, meningioma, mesothelioma, Morton's neuroma, multiple myeloma, myeloid leukemia, myelolipoma, myeloma, myoblastoma, myxoma, nasopharyngeal carcinoma, neuroblastoma, neurofibroma, neuroglioma, neuroma, non-Hodgkin's lymphoma, oligodendroglioma, optic glioma, osteochondroma, osteogenic sarcoma, osteosarcoma, ovarian cancer, pancoast tumor, pancreatic cancer, phaeochromocytoma, plasmacytoma, primary brain tumor, progonoma, prolactinoma, renal cell carcinoma, retinoblastoma, rhabdosarcoma, sarcoma, skin cancer, small cell carcinoma, squamous cell carcinoma, T-cell lymphoma, testicular cancer, thymoma, trophoblastic tumor, and Wilm's tumor.

92. The method of claim 90, wherein said method reduces or prevents pre-cancerous lesions, reduces tumor size or burden, slows or stops the progression of cancer, reduces or stops metastasis, or eliminates cancer cells that have been pushed to senescence.

93. The method of claim 88, wherein said subject has received, is receiving, or will receive a DNA damaging therapy and/or cytotoxic therapy.

94. The method of claim 93, wherein said DNA damaging or cytotoxic therapy comprises a treatment for a cancer selected from the group consisting of leukemia, a secondary tumor, a solid tumor, acute leukemia, adrenal gland tumor, ameloblastoma, anaplastic carcinoma of the thyroid, angioma, apudoma, argentaffinoma, arrhenoblastoma, ascites tumor, astroblastoma, astrocytoma, ataxia-telangiectasia-associated tumors, basal cell carcinoma, bone cancer, brain tumor, brainstem glioma, breast cancer, Burkitt's lymphoma, cervical cancer, cholangioma, chondroblastoma, chondrosarcoma, chorioblastoma, choriocarcinoma, colon cancer, craniopharyngioma, cystocarcinoma, cystofbroma, cystoma, ductal carcinoma, ductal papilloma, dysgerminoma, encephaloma, endometrial carcinoma, endothelioma, ependymoma, erythroleukemia, Ewing's sarcoma, extra nodal lymphoma, fibro adenoma, fibro sarcoma, follicular cancer of the thyroid, ganglioglioma, gastrinoma cell, glioblastoma multiform, glioma, gonadoblastoma, haemangioblastoma, haemangioendothelioblastoma, haemangioendothelioma, haemangiopericytoma, haematolymphangioma, haemocytoblastoma, haemocytoma, hairy cell leukemia, hamartoma, hepatocarcinoma, hepatocellular carcinoma, hepatoma, histoma, Hodgkin's disease, hypernephroma, infiltrating cancer, infiltrating ductal cell carcinoma, insulinoma, juvenile angioforoma, Kaposi sarcoma, kidney tumor, large cell lymphoma, leukemia, lipoma, liver cancer, liver metastases, Lucke carcinoma, lung cancer, lymphadenoma, lymphangioma, lymphocytic leukemia, lymphocytic lymphoma, lymphoedema, lymphoeytoma, lymphoma, malignant mesothelioma, malignant teratoma, mastocytoma, medulloblastome, melanoma, meningioma, mesothelioma, Morton's neuroma, multiple myeloma, myeloid leukemia, myelolipoma, myeloma, myoblastoma, myxoma, nasopharyngeal carcinoma, neuroblastoma, neurofibroma, neuroglioma, neuroma, non-Hodgkin's lymphoma, oligodendroglioma, optic glioma, osteochondroma, osteogenic sarcoma, osteosarcoma, ovarian cancer, pancoast tumor, pancreatic cancer, phaeochromocytoma, plasmacytoma, primary brain tumor, progonoma, prolactinoma, renal cell carcinoma, retinoblastoma, rhabdosarcoma, sarcoma, skin cancer, small cell carcinoma, squamous cell carcinoma, T-cell lymphoma, testicular cancer, thymoma, trophoblastic tumor, and Wilm's tumor.

95. The method of claim 93, wherein said DNA damaging therapy and/or cytotoxic therapy is selected from the group consisting of irradiation, alkylating agents such as nitrogen mustards (chlorambucil, cyclophosphamide, ifosfamide, melphalan), nitrosoureas (streptozocin, carmustine, lomustine), alkyl sulfonates (busulfan), triazines (dacarbazine, temozolomide) and ethylenimines (thiotepa, altretamine), platinum drugs such as cisplatin, carboplatin, oxalaplatin, antimetabolites such as 5-fluorouracil, 6-mercaptopurine, capecitabine, cladribine, clofarabine, cytarabine, floxuridine, fludarabine, gemcitabine, hydroxyurea, methotrexate, pemetrexed, pentostatin, thioguanine, anthracyclines such as daunorubicin, doxorubicin, epirubicin, idarubicin, anti-tumor antibiotics such as actinomycin-D, bleomycin, mitomycin-C, topoisomerase inhibitors such as topoisomerase I inhibitors (topotecan, irinotecan) and topoisomerase II inhibitors (etoposide, teniposide, mitoxantrone), mitotic inhibitors such as taxanes (paclitaxel, docetaxel), epothilones (ixabepilone), vinca alkaloids (vinblastine, vincristine, vinorelbine), estramustine, cyclin-dependent kinase inhibitors (roscovitine, palbociclib, abemaciclib, olaparib), epigenetic modifiers (curcumin, valproic acid), and HIV medications such as NRTIs (Nucleoside Reverse Transcriptase Inhibitors), NNRTIs (Non-Nucleoside Reverse Transcriptase Inhibitors), and protease inhibitors (azidothymidine, tenofovir, emtricitabine, abacavir, nevirapine, atazanavir, lopinavir).

96. The method of claim 88, wherein said method (a) delays the onset, slows, and/or stops the progression of one or more symptoms associated with accumulation of senescent cells from said DNA damaging therapy; (b) delays the onset, slows, and/or stops the progression of one or more features of aging in the subject; or (c) reduces the severity, ameliorates one or more symptoms, and/or delays the onset, slows, and/or stops the progression of a senescence-associated disease or disorder.

97. The method of claim 96, wherein said one or more features of aging is selected from the group consisting of systemic decline of the immune system, muscle atrophy and decreased muscle strength, decreased skin elasticity, delayed wound healing, retinal atrophy, reduced lens transparency, reduced hearing, osteoporosis, sarcopenia, hair graying, skin wrinkling, poor vision, frailty, cognitive impairment, ophthalmic disease, and idiopathic pulmonary fibrosis.

98. The method of claim 96, wherein the senescence-associated disease or disorder is selected from the group consisting of cardiovascular disease; Alzheimer's disease and related dementias; Parkinson's disease; Huntington's disease; mild cognitive impairment; motor neuron dysfunction; cataracts; macular degeneration; glaucoma; presbyopia; atherosclerosis; acute coronary syndrome; myocardial infarction; stroke; hypertension; idiopathic pulmonary fibrosis (IPF); chronic obstructive pulmonary disease (COPD); asthma; cystic fibrosis; emphysema; bronchiectasis; age-related loss of pulmonary function; osteoarthritis; osteoporosis; obesity; fat dysfunction; coronary artery disease; cerebrovascular disease; periodontal disease; cancer treatment-related disabilities including atrophy and fibrosis in tissues, brain and heart injury, or therapy-related myelodysplastic syndromes; an accelerated aging disease including progeroid syndromes, Hutchinson-Gilford progeria syndrome, Werner syndrome, Bloom syndrome, Rothmund-Thomson Syndrome, Cockayne syndrome, trichothiodystrophy, combined xeroderma pigmentosum-Cockayne syndrome, or restrictive dermopathy; ataxia telangiectasia, Fanconi anemia; Friedreich's ataxia; dyskeratosis congenital; aplastic anemia; IPF; renal dysfunction; renal disease; renal failure; skin wound healing; liver fibrosis; pancreatic fibrosis; oral submucosa fibrosis; inflammatory bowel disease; kyphosis; herniated intervertebral disc; frailty; hair loss; hearing loss; vision loss including blindness or impaired vision; muscle fatigue; diabetes; diabetic ulcer; metabolic syndrome; sarcopenia; sarcopenia oral mucositis; and dermatological conditions including wrinkles, superficial fine wrinkles, hyperpigmentation, scars, keloid, dermatitis, psoriasis, eczema, seborrheic eczema, rosacea, vitiligo, ichthyosis vulgaris, dermatomyositis, nevi, rashes, atopic dermatitis, urticaria, rhytides, pruritis, dysesthesia, eczematous eruptions, eosinophilic dermatosis, reactive neutrophilic dermatosis, pemphigus, pemphigoid, immunobullous dermatosis, fibrohistocytic proliferations of skin, cutaneous lymphomas, cutaneous lupus, and actinic keratosis.

99. The method of claim 96, wherein the senescence-associated disease or disorder is a cardiovascular disease selected from the group consisting of atherosclerosis, angina, arrhythmia, cardiomyopathy, congestive heart failure, coronary artery disease, carotid artery disease, endocarditis, coronary thrombosis, myocardial infarction, hypertension, aortic aneurysm, cardiac diastolic dysfunction, hypercholesterolemia, hyperlipidemia, mitral valve prolapsed, peripheral vascular disease, cardiac stress resistance, cardiac fibrosis, brain aneurysm, and stroke.

100. The method of claim 99, wherein said method comprises ameliorating a symptom selected from the group consisting of irregularity in heart rhythm, age-related cellular hypertrophy, increase in the cross-sectional area of a cardiomyocyte and decrease in cardiac stress tolerance.

101. The method of claim 88, wherein said dihomo-gamma-linolenic acid (DGLA), and/or gamma-linolenic acid (GLA), and/or delta-5-desaturase inhibitor (D5D inhibitor) is administered orally, systemically, topically, transdermally, intradermally, intranasally, by inhalation, intratracheally, by intubation, or directly to an organ or tissue that comprises the senescent cells.

102. The method of claim 88, wherein said subject is a human or non-human mammal.

103. The method of claim 88, wherein said subject has a pathology characterized by the generation of senescent cells and an inflammatory response.

104. The method of claim 103, wherein said pathology comprises kyphosis, herniated intervertebral discs, osteoporosis, irritable bowel syndrome, an inflammatory bowel disease, colitis, Crohn's disease, a pulmonary disease, idiopathic pulmonary fibrosis (IPF), chronic obstructive pulmonary disease (COPD), asthma, cystic fibrosis, bronchiectasis, emphysema, a pathology characterized by fibrosis, renal fibrosis, liver fibrosis, pancreatic fibrosis, cardiac fibrosis, skin wound healing, oral submucous fibrosis, or a combination thereof.

105. The method of claim 88, wherein said agent comprises the DGLA.

106. The method of claim 105, wherein said DGLA is provided as DGLA ethyl ester, DGLA inert lipid, or a combination thereof.

107. The method of claim 105, wherein said DGLA is administered without administration of a D5D inhibitor or GLA to said subject.

108. The method of claim 88, wherein said agent comprises the GLA.

109. The method of claim 108, wherein said GLA is administered without administration of a D5D inhibitor to said subject.

110. The method of claim 88, wherein said agent comprises the D5D inhibitor.

111. The method of claim 110, wherein said D5D inhibitor comprises D5D-IN-326 (2-(2,2,3,3,3-Pentafluoropropoxy)-3-[4-(2,2,2-trifluoroethoxy) phenyl]-5,7-dihydro-3H-pyrrolo[2,3-d]pyrimidine-4,6-dione, CAS No.: 1236767-85-3), CP 24,879, (4-(3-methylbutoxy)-benzenamine, monohydrochloride), T3364366 (N-[2-[[3,4-Dihydro-4-oxo-3-[4-(2,2,2-trifluoroethoxy)phenyl]thieno[3,4-d]pyrimidin-2-yl]thio]ethyl]acetamide), or an inhibitor selected from the group consisting of iminodibenzyl, iminostilbene. compound 1a, compound 3a, compound 1b, compound 3b, compound 1d, compound 1e, compound 1f, compound 2e, compound 3e, compound 2f, compound 3f, compound as shown in Table 1, any one or more of compounds 1-354 as shown in Table 2, and/or compound 326 described by Takagahara et al.

112. The method of claim 88, wherein said subject is not diagnosed with and/or under treatment for a pathology characterized by aggregation of a protein selected from the group consisting of Aβ, tau, and alpha-synuclein.

113. The method of claim 88, wherein said subject is not under treatment for a neurological pathology, an ophthalmic disorder, Alzheimer's disease and related dementias, amyloid or other cause-mediated mild cognitive impairment (MCI), brain or spinal cord injury, stroke, Huntingtin's disease, Parkinson's disease, or a skin pathology selected from the group consisting of systemic sclerosis, psoriasis, and eczema.

114. The method of claim 88, wherein said DGLA is not administered (a) for the treatment of rheumatoid arthritis (RA) and/or to a subject diagnosed with RA; (b) for the treatment of polyps in the mouth, to a subject diagnosed with polyps in the mouth, and/or to a subject identified as having polyps in the mouth; (c) for the treatment of high cholesterol and/or other blood fats, and/or to a subject identified as having high cholesterol and/or other blood fats; (d) for the treatment of heart disease and/or to a subject identified as having heart disease; (e) for the treatment of metabolic syndrome (Syndrome-X) and/or to a subject identified as having metabolic syndrome; (f) for the treatment of diabetic nerve pain or damage and/or to a subject identified as having diabetic nerve pain or damage; (g) for the treatment of attention deficit-hyperactivity disorder (ADHD) and/or to a subject identified as having ADHD; (h) for the treatment of depression and/or depression after childbirth, and/or to a subject identified as having depression and/or depression after childbirth; (i) for the treatment of chronic fatigue syndrome (CFS) and/or to a subject identified as having CFS; or (j) for the treatment of hay fever (allergic rhinitis) and/or to a subject identified as having hay fever.

115. The method of claim 88, wherein said DGLA is not administered to help breast cancer patients respond faster to treatment with the drug tamoxifen.

116. The method of claim 88, wherein said DGLA and/or said GLA is not administered as a dietary component or as a nutraceutical, or wherein said DGLA and/or GLA is not provided as a plant seed and/or plant seed oil.

117. The method of claim 88, wherein said method does not comprise administration of DGLA and/or GLA in conjunction with a D5D inhibitor for treatment of a cancer or precancerous condition, an autoimmune condition, or an inflammatory pathology.

118. The method of claim 88, wherein said DGLA, GLA, and/or D5D inhibitor is administered in conjunction with one or more additional senolytic agents.

119. The method of claim 118, wherein said additional senolytic agents comprise one or more of a CRYAB inhibitor, a senolytic agent described in U.S. Patent Publication Nos: US 2019/0022090, US 2019/0000846, US 2018/0303828, US 2018/0256568, US 2018/0235957, US 2018/0235956, US 2018/0193458, US 2018/0117038, US 2017/0348307, US 2017/0326136, US 2017/0224680, US 2017/0209435, US 2017/0198253, US 2017/0196858, US 2017/0196857, US 2016/0339019, US 2016/0038576, an MDM2 inhibitor, an inhibitor of one or more BCL-2 anti-apoptotic protein family members wherein the inhibitor inhibits at least BCL-xL BCL2, a benzothiazole-hydrazone compound, an aminopyridine compound, a benzimidazole compound, a tetrahydroquinolin compound, a phenoxyl compound, and/or an Akt-specific inhibitor.
